SEQUENCE LISTING (5 sequences)

SEQ ID NO: 1 - DNA, 20 bp, Artificial
  Synthetic oligonucleotide
cgttgtaaaacgacggccag

SEQ ID NO: 2 - DNA, 20 bp, Artificial
  Synthetic oligonucleotide
ctggccgtcgttttacaacg

SEQ ID NO: 3 - DNA, 20 bp, Artificial
  Synthetic oligonucleotide
ctaactgttacctcggtcgg

SEQ ID NO: 4 - DNA, 21 bp, Artificial
  Synthetic oligonucleotide
caatacgcaaaccgcctctcc

SEQ ID NO: 5 - DNA, 22 bp, Artificial
  Synthetic oligonucleotide
tcggtgatgtcggcgatatagg

(19) United States
(12) Patent Application Publication
(10) Pub. No.: US 2007/0072199 A1
(43) Pub. Date: Mar. 29, 2007

(54) Covalent methods for immobilization of thiolated biomolecules on siliceous and metallic surfaces

(75) Inventors: Rastislav Levicky, Irvington, NY (US); Patrick A. Johnson, New York, NY (US); Adrian Horgan, Essex (GB)

Correspondence Address: WilmerHale/Columbia University, 399 PARK AVENUE, NEW YORK, NY, 10022, US

(73) Assignee: THE TRUSTEES OF COLUMBIA UNIVERSITY IN THE CITY OF NEW YORK, New York, NY (US)

(21) Appl. No.: 11/316,566

(22) Filed: Dec. 22, 2005

Related U.S. Application Data
(63) Continuation of application No. PCT/US04/20355, filed on Jun. 22, 2004.
(60) Provisional application No. 60/482,063, filed on Jun. 24, 2003.

(51) Int. Cl. C12Q 1/68

(52) U.S. Cl. 435/6; 427/2.11; 257/E51.02

(57) ABSTRACT

The present invention is directed to methods for immobilizing molecules on siliceous and metallic surfaces. Molecules are immobilized on siliceous or metallic surfaces by stable covalent linkages that are capable of withstanding prolonged use and elevated temperatures. Further, the methods of the present invention describe less-complicated chemistries for the immobilization of molecules that will benefit the reproducibility, efficiency and effectiveness of applications in sensing, chromatography, medical diagnostics and related areas where specific recognition between immobilized and free molecules provides diagnostic information or serves as part of a purification or separations process.

This application is a continuation of PCT Intl. Appln. No. PCT/US2004/020355, filed Jun. 22, 2004 and claims the benefit of provisional application U.S. Ser. No. 60/482,063, filed Jun. 24, 2003, which are hereby incorporated by reference into the subject application in their entireties.

The invention disclosed herein was made with U.S. Government support from the National Science Foundation ((DMR-00-93758), (DMR-0213574) and (ECS-99-80828)) Accordingly, the U.S. Government has certain rights in this invention.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

Copyright Statement: copyright in the text and graphic materials contained in this disclosure is owned by Columbia University of New York. The materials contained in this disclosure may be used, downloaded, reproduced or reprinted, provided that this copyright notice appears in all copies and provided that such use, download, reproduction or reprint is for noncommercial or personal use only. The materials contained in this disclosure may not be modified in any way.

### SUMMARY OF THE INVENTION

The present invention is directed to methods for immobilizing molecules on siliceous and metallic surfaces. The methods of the present invention provide stable covalent linkages of molecules immobilized on siliceous or metallic surfaces that are capable of withstanding prolonged use or elevated temperatures. Further, the methods of the present invention describe less-complicated chemistries for the immobilization of molecules that will benefit the reproducibility, efficiency and effectiveness of applications related to the present invention.

The immobilization of molecules on siliceous and metallic surfaces relates to applications in sensing, chromatography, medical diagnostics and related areas where specific recognition between immobilized and free molecules provides diagnostic information or serves as part of a purification or separations process.

The chemistry employed for immobilization of molecules on solid supports that comprise siliceous and metallic surfaces is important because the chemistry impacts the activity and permanence of the surface-tethered layer(s), i.e., the layer(s) of immobilized molecules.

For immobilization to siliceous surfaces, the present invention relates to the preparation of maleimide-activated siliceous surfaces that enable surface conjugation of thiolated molecules. The preparation of maleimide-activated siliceous surfaces first involves derivatizing or silylating a siliceous substrate that has a silanol surface. The silylation involves an aminosilane, in one embodiment, aminopropyltriethoxysilane (APTES), that results in the introduction of amine groups to the surface of the siliceous substrate. In a second step, a heterobifunctional crosslinking reagent is used that reacts with the amine groups and enriches the siliceous surface with maleimide moieties. In a third step, sulfhydryl (thiol) containing molecules are attached or immobilized to the surface via thioether bonds to the maleimides.

For immobilization to metal surfaces, the present invention relates to the preparation of metal-substrate modified to have a surface with thiol groups that enable surface conjugation to molecules with maleimide groups. For this method, a first step involves the self-assembly of a monolayer of a thiol-derivatized polysiloxane, in one embodiment poly(mercaptopropyl)methylsiloxane (PMPMS), to a metal surface, such as gold. Multivalent binding of the polysiloxane thiols to the gold, combined with the polysiloxane's hydrophobic nature, causes it to irreversibly adhere to the metal surface. Maleimide containing molecules, such as maleimide-terminated DNA oligonucleotides, are subsequently covalently linked to the PMPMS modified metal film or surface via thioether bonds.

In another aspect of the present invention, the molecules that are immobilized to metal surfaces have thiol groups rather than maleimide groups. The first step involves the self-assembly of a monolayer of a thiol-derivatized polysiloxane, in one embodiment poly(mercaptopropyl)methylsiloxane (PMPMS), to a metal surface, such as gold. Thiol groups on the metal surface are then reacted with a bismaleimide crosslinker, where one maleimide end of the crosslinker forms a thioether linkage with the metal surface, resulting in a metal surface with free maleimide groups. Thiolated molecules are then immobilized to the metal surface through the formation of thioether bonds.

### BRIEF DESCRIPTION OF THE DRAWINGS

FIG. 1. Transmission electron micrograph of Aerosil® 200 (JEOL JEM-100C, 100 keV).

FIG. 2. DNA immobilization involved (i) silanization of the solid support with APTES, (ii) reaction of crosslinker (PMPI, MBS, or sulfo-MBS) with APTES to generate a maleimide surface, and (iii) reaction of thiol endgroups on DNA with surface maleimides. The isocyanate group of PMPI (shown) forms a urea linkage with APTES amines. The NHS-ester moieties on MBS and sulfo-MBS (not shown) would form an amide linkage.

FIG. 3. Representative mid-IR spectra of fumed silica at various stages of modification: (a) neat, (b) after APTES modification, and after attachment of (c) PMPI or (d) MBS linker. Asterisks indicate peaks chosen for quantification of silica (a), APTES (b), PMPI (c), and MBS (d).

FIG. 4. Effect of silane concentration in bulk solution on realized surface coverage of APTES on Aerosil® 200. The x-axis represents the number of APTES molecules added to solution per nm² of silica surface present, with a unit of 1 corresponding to APTES concentration of 8.9 mM.

FIG. 5. Surface coverage of PMPI residues (filled circles) and active maleimide groups (open circles) as a function of PMPI solution concentration reacted with the silica. A solution to surface excess of 1 molecule/nm² corresponds to 13 mM PMPI in acetonitrile.

FIG. 6. Left: The bismaleimide N,N′-bis(p-maleimidophenyl)urea form by water-mediated condensation of two PMPI molecules. Right: Michael addition between one end of the bismaleimide and a silane amine leads to two PMPI residues and one active maleimide on the surface.

FIG. 7. Surface coverage of MBS residues (filled circles) and active maleimide groups (open circles) as a function of MBS solution concentration used to modify APTES-silica. A solution to surface excess of 1 molecule/nm² corresponds to 9.5 mM MBS in acetonitrile.

FIG. 8. Surface coverage of PSH and P oligonucleotides as a function of bulk concentration (1 μM or 0.1 μM) used for the attachment.

FIG. 9. Sequence-specificity and extents of hybridization of PSH layers at two different coverages of bound oligonucleotide: 2.1×10¹³ and 2.2×10¹² strands/cm². The percentage of PSH oligonucleotides that hybridized to TC targets is shown on the corresponding columns.

FIG. 10. Topological constraints prevents hybridization of a multiply attached surface strand. If the spacing between crosslinks is incompatible with that required to accommodate a double helix, then hybridization will be suppressed.

FIG. 11. Three steps in a method for the preparation of DNA functional surfaces.

FIG. 12. Silica modification with APDMES.

FIG. 13. PMPI reacts with water to form N,N′-bis(p-maleimidophenyl)urea.

FIG. 14. Introduction of maleimide groups to an aminosilanized surface with the crosslinking reagent PMPI.

FIG. 15. Structural images of MBS (top) and Sulfo-GMBS (bottom).

FIG. 16. Attachment of thiol-terminated oligonucleotides to surface maleimide groups (illustrated for a PMPI derivatized surface).

FIG. 17. Elution of 20mer oligonucleotide, DTT, TCEP, and NaCl through a PD-10 column.

FIG. 18. Assembly used to hold powder Aerosil® 200 samples for FTIR measurement.

FIG. 19. A UV-Vis absorption trace of DNA in phosphate buffer.

FIG. 20. Structure and reaction of Ellman's Reagent (DTNB).

FIG. 21. Structure of L-cysteine.

FIG. 22. Modification of fumed silica with thiol-terminated DNA oligonucleotides. The isocyanate group of PMPI (shown) forms a urea link with surface amines. The NHS-ester sites on MBS and sulfo-GMBS (not shown) form an amide link.

FIG. 23. Infrared spectrum of Aerosil® 200 (pressed disk specimen).

FIG. 24. Infrared spectra of Aerosil® 200 pressed disks after the indicated heat treatments at 900° C.

FIG. 25. Infrared spectra of Aerosil® silica between 1950 to 1750 cm⁻¹. These spectra were used to determine the silica extinction coefficient.

FIG. 26. Calibration plot of integrated infrared absorption (1950 to 1750 cm⁻¹) vs. amount of Aerosil® 200 present in the beam.

FIG. 27. Infrared spectra of (3-Aminopropyl)dimethylethoxysilane in CCl₄.

FIG. 28. Infrared spectrum of (3-Aminopropyl)triethoxysilane on Aerosil® 200.

FIG. 29. Molecular structure of free APDMES (left), surface-bound APDMES (center), and BAPTDS (right).

FIG. 30. Spectra of APDMES solutions in CCl₄ for three different concentrations (after subtraction of a linear baseline from 3025 to 2775 cm⁻¹). Concentrations are indicated both as v/v percentages and as corresponding mass amounts of silane in the infrared beam (calculated from density of APDMES of 0.839 g/cm³ and dimensions of the liquid cell which was 3 mm diameter and 1 mm thick). The integrated area for each set of peaks is also listed.

FIG. 31. Spectra of BAPTDS solutions in CCl₄ for three different concentrations (after subtraction of a linear baseline from 3025 to 2775 cm⁻¹). Concentrations are indicated both as v/v percentages and as corresponding mass amounts of silane in the infrared beam (calculated from density of BAPTDS of 0.856 g/cm³ and dimensions of the liquid cell which was 3 mm in diameter and 1 mm thick). The integrated area for each set of peaks is also listed.

FIG. 32. Integrated infrared absorbance of APDMES (3050 cm⁻¹ to 2750 cm⁻¹) in CCl₄ vs. amount of silane present in the IR beam.

FIG. 33. Integrated infrared absorbance of BAPTDS (3050 cm⁻¹ to 2750 cm⁻¹) in CCl₄ vs. amount of silane present in the IR beam.

FIG. 34. Calibration of APDMES and APTES infrared absorbance to absolute surface densities of silane determined from elemental analysis. Lines are linear fits to the data (see relations 3.1 and 3.2).

FIG. 35. Transmission IR spectrum of PMPI in CCl₄.

FIG. 36. Transmission IR spectrum of PMPI on APTES-modified Aerosil® 200 powder.

FIG. 37. Left: Molecular structure of PMPI. Right: Schematic of a silica surface modified with APTES in a first step followed by reaction with PMPI.

FIG. 38. Calibration of PMPI infrared absorbance to absolute surface densities as determined from elemental analysis. The line is a linear fit to the data (relation 3.3).

FIG. 39. Infrared spectrum of MBS on APTES modified Aerosil®.

FIG. 40. Left: Molecular structure of MBS. Right: Schematic of a silica surface modified with APTES in a first step followed by reaction with MBS.

FIG. 41. Calibration of MBS infrared absorbance to absolute surface densities as determined from elemental analysis. The line is a linear fit to the data (relation 3.4).

FIG. 42. IR spectra of pressed Aerosil® 200 disks as a function of reaction time with 3% v/v APDMES in toluene.

FIG. 43. Remnant silane coverage after subjecting APDMES modified Aerosil® to different number of ethanol washes.

FIG. 44. Mid-IR spectra of fumed silica (a) after APTES modification, and (b) after attachment of PMPI.

FIG. 45. Left: Molecular structure of NPM. Right: Schematic of a silica surface modified with APTES in a first step followed by reaction with NPM.

FIG. 46. Infrared spectrum of Aerosil® 200 that was first modified with APTES, and subsequently reacted with NPM.

FIG. 47. Mid-IR spectra of fumed silica (a) after APTES modification and (b) after attachment of MBS.

FIG. 48. Illustration of an APTES-modified silica after reaction with MBS (top) and PMPI (bottom). MBS linkers tend to react at both ends, while PMPI strongly selects reaction via the isocyanate (—NCO) group.

FIG. 49. Surface coverage of active MBS maleimide groups on Aerosil® 200 as a function of time after attachment.

FIG. 50. IR spectra of PMPI-activated silica after 1-hour immersion in deionized water at the indicated temperature.

FIG. 51. Residual coverage of PMPI residues after 1-hour immersion in deionized water as a function of temperature.

FIG. 52. Confocal fluorescence microscopy images showing hybridization of P and PSH oligonucleotides to complementary (TC) and noncomplementary (TNC) targets. The PSH or P strands were immobilized on APTES covered glass slides activated with PMPI crosslinkers. Left: before hybridization. Middle: after hybridization to TC targets. Right: after hybridization to TNC targets.

FIG. 53. Schematic diagram of the reaction cell used for functionalization of glass slides.

FIG. 54. Oligreen® standard curve measured using a polystyrene cuvette.

FIG. 55. Oligreen® standard curve measured using a quartz cuvette.

FIG. 56. Immobilization of oligonucleotides on gold surfaces. (a) Conventional approach based on a direct reaction of a thiol moiety on the molecule of interest with the metal support. Short chain alkanethiols are used to “fill in” the intervening surface area and thus to control biomolecule-surface interactions (for example, the endgroup X may be a hydroxyl). (b) Present method using a chemisorbed layer of PMPMS polymer to introduce surface thiol groups, followed by attachment of maleimide-terminated DNA oligonucleotides.

FIG. 57. X-ray photoelectron spectroscopy (XPS) spectra from a PMPMS monolayer reacted with DNA-S-BM(PEO)₄: (a) C 1s; (b) S 2p; (c) N 1s; (d) P 2p. Deconvolution of unprocessed raw data (filled circles) into separate components (dashed lines) is indicated where applicable. Solid lines show fitted peak sums and baseline subtractions. The residuals between raw data and calculated fits are shown at the bottom of each plot. In (b), dotted lines are the double-peak of the thiolate component (see text) while dashed lines represent photoelectron intensity from free thiols and disulfides.

FIG. 58. Voltammetric response of bare Au (solid line), Au modified with PMPMS (dashed line), and Au modified with MCH (dash-dotted line). The electrodes were preconditioned through 9 cycles before measurement of the above data. Electrolyte: 100 mM potassium phosphate, pH 10. Sweep rate: 50 mV/sec. Electrode area: 0.54 cm².

FIG. 59. Changes in XPS P 2p and N 1s signals after hybridization of PMPMS surfaces functionalized with DNA-S-BM(PEO)₄ to complementary and noncomplementary oligonucleotide sequences.

FIG. 60. Changes in XPS signals following immersion of sample in 95° C. buffer for 1 hour for (a) oligonucleotides attached via terminal thiols with mercaptohexanol passivation according to reference¹⁷ of Example 4 and, (b) present method utilizing a PMPMS anchor film.

FIG. 61. Attachment of LUC-MAL to PMPMS anchor film.

FIG. 62. Raw P 2p traces from PMPMS films reacted with LUC amplicons. The inset schematics illustrate the tested mechanism of attachment (see text). All data are for 36 hours immobilization from ˜1×10⁻⁸ M solutions of the DNA in 0.015 M sodium citrate, 1.0 M NaCl, pH 7.0.

FIG. 63. Integrated P 2p intensity from LUC-MAL monolayers following immersion in hot buffer (1.5 mM sodium citrate, pH 7) at the indicated ionic strength.

FIG. 64. Attachment of DNA gene monolayer to a PMPMS polymer adhesion layer.

FIG. 65. (a) Maleimide-terminated chains, LUC-MAL, were prepared from disulfide-terminated LUC-S-SR precursors in two steps: disulfide reduction with dithiothreitol (DTT) followed by addition to maleimide olefinic bond on BMPEO4. (b) Attachment of LUC-MAL to gold supports involved chemisorption of a PMPMS layer in a first step, followed by reaction of LUC-MAL with remnant PMPMS thiols to form thioether linkages.

FIG. 66. An overview of surface modification. (i) Silylation of a silica or glass surface with APTES to introduce amine groups, (ii) conversion of the amine-functional into a maleimide-derivatized surface by reaction of APTES residues with PMPI, and (iii) attachment of thiol-terminated DNA via the maleimide olefinic bond. After reference (Jin).

FIG. 67. Squares, circles: Loss of maleimide activity on silica/APTES/PMPI supports as a function of storage under PB (10 mM sodium phosphate, 0.1 M NaCl, pH 7.0) for high (squares) and low (circles) coverages of PMPI. Stars: Bulk solution hydrolysis of the bismaleimide BM(PEO)₄, also under neutral buffer. All points represent an average of two measurements.

FIG. 68. Mid-IR spectra of unmodified silica powder (curve 1), after reaction with APTES (curve 2), and after further derivatization with PMPI (curve 3).

FIG. 69. Infrared absorbance spectra of silica/APTES/PMPI supports in the maleimide and aromatic C—H stretch region. The maleimide C—H band is at 3103 cm⁻¹ (arrow in part (a)). (a) As a function of immersion time in PB buffer at pH 7.0. (b) As a function of a 3 h immersion in different pH buffers (10 mM sodium phosphate of the indicated pH, 0.1 M NaCl).

FIG. 70. Infrared absorbance spectra of silica/APTES/PMPI supports in the carbonyl stretch region. Conditions are as in FIG. 4. Arrows in part (a) indicate maleimide bands whose intensity decreased with longer immersion time (a) or with elevated pH (b).

FIG. 71. XPS C 1s traces from modified glass slides. The magnitude of the traces was normalized to facilitate comparison of peak shape.

FIG. 72. Raw P 2p intensity from APTES/PMPI slides reacted for 5 days with 1.0×10⁻⁶ M oligonucleotide (P1 or P2) solution in citrate buffer (0.015 M sodium citrate, 1 M NaCl, pH). The surfaces were washed with deionized water and dried before characterization by XPS.

FIG. 73. FTIR spectra of functionalized silica powders. 1: neat silica. 2: silica after APTES modification. 3: silica after modification with APTES and NPM. Cross-hatched areas on curve 3 indicate integration peaks used to calculate APTES coverage via equation (1). Inset: Transmission electron micrograph of a grain of fumed silica (image width: 640 nm).

FIG. 74. Possible reaction mechanisms between APTES-modified silica and NPM.

FIG. 75. Left: attachment geometry for NPM and PMPI. PMPI maleimide hydrogens are indicated in bold. Right: C—H stretching region for APTES-modified powders functionalized with PMPI (solid line) and NPM (dashed line). The maleimide C—H band is strongly suppressed in the case of NPM.

FIG. 76. (a) IR spectra of NPM-derivatized silica stored under pH 7 PBS at room temperature. Solid line: 0.5 h; dashed line: 72 h. (b) IR spectra after storage for 2 h in pH 7 PBS at elevated temperatures. Solid line: 30° C.; dashed line: 90° C. Direction of arrow indicates decrease/increase of the respective band. Spectra were normalized for amount of powder by scaling to the 1820-1920 cm⁻¹ silica overtone band.

FIG. 77. Correlation, over seven independent samples, between NPM coverage from elemental analysis (x-axis) and integrated intensity of the 1500 cm⁻¹ aromatic C═C stretch. The aromatic band intensity (1481 to 1510 cm⁻¹) was divided by that of silica (1820 to 1920 cm⁻¹) to normalize for amount of powder used in measurement. Baseline correction was applied as illustrated in the insets, with shaded areas indicating integrated regions.

FIG. 78. (a) Variation of APTES and NPM coverage with storage under PBS buffer at room temperature. APTES coverage (filled circles) was calculated from equation (7.1). NPM to APTES ratio (open circles) was calculated by dividing integrated absorbance of an NPM phenyl band (1481 to 1510 cm⁻¹) by that of APTES C—H stretch bands (2800 to 3000 cm⁻¹). (b) As in (a), but following a 2 hour immersion in PBS buffer at the indicated temperature. All measurements were performed in duplicate, with error bars indicating the standard deviation.

### DETAILED DESCRIPTION OF THE INVENTION

The issued U.S. patents, published and allowed applications, and references cited herein are hereby incorporated by reference.

The present invention relates to methods for immobilizing molecules on siliceous or metal surfaces where the chemistry of the methods provide stable covalent linkages of molecules immobilized on siliceous or metallic surfaces that are capable of withstanding prolonged use or elevated temperatures.

The term “siliceous” as used herein refers to substances relating to, or containing silica (in any of its forms, i.e., crystalline, amorphous, impure, fused, fumed), silicate, or silica gel.

The term “surface” as used herein refers to the exterior or external part or layer of an object or molecule.

The term “substrate” as used herein refers to a substance that is acted upon.

The term “immobilized” as used herein refers to molecules that are attached to the surface of siliceous or metal substrates.

The term “crosslinker” as used herein refers to a molecule that may form a covalent linkage with at least one other molecule or mediate a covalent linkage between two molecules or between two different regions of the same molecule.

The term “heterobifunctional crosslinker” as used herein refers to crosslinkers that possess more than one (typically two) reactive sites designed to covalently react with different moieties, such as an amine at one site and a thiol at the other.

The present invention provides methods for immobilizing molecules on a siliceous surface comprising the steps of (a) silylating a siliceous substrate comprising a silanol surface with an aminosilane thereby forming a modified siliceous substrate comprising an aminosilanized surface; (b) reacting the aminosilanized surface with a heterobifunctional crosslinker, thereby forming a further modified siliceous substrate comprising a maleimide surface; and (c) reacting the maleimide surface with thiolated molecules wherein the molecules comprise terminal thiol groups wherein the reaction between the maleimide surface and the terminal thiol groups form thioether linkages, thereby forming a siliceous surface comprising immobilized molecules.

The siliceous substrates of the present invention include, but is not limited to, amorphous silica, fumed amorphous silica or fused silica. Other types of silica may be used as a siliceous substrate, such as hydrophilic silica, hydrophobic silica, and crystalline silica.

The aminosilanes of the present invention include, but are not limited to, (3-aminopropyl)dimethylethoxysilane (APDMES), (3-aminopropyl)triethoxysilane (APTES), and short alkyl triochlorosilane derivates (H₂N—(CH₂)ₓ—SiCl₃). Other silanes, such as long-chain trichlorosilanes, tetramethoxysilane (TMOS), tetraethoxysilane (TEOS), tetrapropoxysilane, methyltrimethoxysilane, methyltriethoxysilane, methyltris(methylethylketoxime)silane (MOS), methyltris(acetoxime)silane, methyltris(methylisobutylketoxime)silane, dimethyldi(methylethylketoxime)silane, trimethyl(methylethylketoxime)silane, vinyltris(methylethylketoxime)silane (VOS), methylvinyldi(methylethylketoxime)silane, methylvinyldi(cyclohexanoneoxime)silane, vinyltris(methylisobutylketoxime)silane, phenyltris(methylethylketoxime)silane (POS), methyltriacetoxysilane, and tetracetoxysilane, may be used as long as amine functionality is incorporated. In one embodiment, the aminosilane is APTES or APDMES. In another embodiment, the aminosilane is APTES.

Heterobifunctional crosslinkers include, but are not limited to, N-(ρ-maleimidophenyl)isocyanate (PMPI), m-maleimidobenzoyl-N-hydroxysuccinimide ester (MBS) or N-(γ-maleimidobutyryloxy)sulfosuccinimide ester (sulfo-GMBS). In one embodiment, the heterobifunctional crosslinker is PMPI.

Molecules that may be immobilized by the methods of the present invention include, but are not limited to, nucleic acids, peptides, proteins, lipids, and sugars.

The present invention provides methods for immobilizing molecules on a metal-film surface that comprise the steps of (a) attaching a thiol-derivatized polysiloxane, such as poly(mercaptopropyl)methylsiloxane (PMPMS) on a metal-film substrate thereby forming a modified metal-substrate comprising a thiol surface; (b) reacting the thiol surface with a bismaleimide crosslinker, thereby forming a further modified metal-film substrate comprising a maleimide surface; and (c) reacting the maleimide surface with thiolated molecules, wherein the molecules comprise thiol groups, and wherein the reaction between the maleimide surface and the thiol groups form thioether linkages, thereby forming a metal-film substrate comprising immobilized molecules.

The present invention also provides methods for immobilizing molecules on a metal-film surface that comprise the steps of (a) attaching a thiol-derivatized polysiloxane such as PMPMS on a metal-film substrate thereby forming a modified metal-substrate comprising thiol group; and (b) reacting the modified metal-substrate with maleimide-modified molecules comprising maleimide groups, wherein the reaction between the thiol groups of the modified metal-substrate and the maleimide groups form thioether linkages, thereby forming a metal-film surface comprising immobilized molecules.

The metal-film substrate includes metals such as, for example, cadmium, chromium, cobalt, copper, gold, hafnium, iridium, iron, manganese, mercury, molybdenum, nickel, niobium (columbium), osmium, palladium, platinum, rhenium, rhodium, ruthenium, scandium, silver, tantalum, technetium, titanium, tungsten, vanadium, yttrium, zinc and zirconium. In one embodiment, the metal-film substrate comprises gold.

It is to be understood and expected that variations in the principles of invention herein disclosed in exemplary embodiments may be made by one skilled in the art and it is intended that such modifications, changes, and substitutions are to be included within the scope of the present invention.

### EXAMPLE 1

### Preparation of End-Tethered DNA Monolayers on Siliceous Surfaces Using Heterobifunctional Crosslinkers

Modification of solid supports with biomolecules (nucleic acids, peptides) finds application in sensing, chromatography, medical diagnostics, and related areas where specific recognition between immobilized and free species provides diagnostic information or serves as part of a purification or separations process. The chemistry employed for immobilization of biomolecules to the solid support is important as it impacts the activity and permanence of the surface-tethered layer. Maleimide-activated siliceous surfaces are often employed for surface conjugation of thiolated biomolecules. Each chemical step involved was validated. Use of high surface area fumed silica as the solid support enabled application of standard analytical techniques of infrared spectroscopy, elemental analysis, and titration assays to the modified surfaces. Silica derivatized with aminopropyltriethoxysilane (APTES) in a first step was then activated with heterobifunctional crosslinkers to introduce maleimide groups. The crosslinkers bear an amine-reactive site, either isocyanate or N-hydroxysuccinimide ester (NHS-ester), in addition to a thiol-reactive maleimide. The amine-reactive site reacts with the surface (e.g. APTES) amines while retaining maleimide activity for subsequent reaction with thiols (e.g. on a biomolecule).

Isocyanate-containing crosslinkers yield surfaces highly active in maleimide groups, and demonstrate subsequent immobilization and hybridization of DNA oligonucleotides. In contrast, under comparable conditions, NHS-ester crosslinkers were less effective due to deactivation of their maleimide groups by side reaction with surface amines. The coverage of each species, APTES and crosslinker, and thus the stoichiometry of their reaction was also determined.

### Introduction of Example 1

Nucleic acids immobilized on solid supports are widely employed in biological and medical diagnostics (1-3). They also provide fundamental insight into chemically and physically adsorbed polymer layers (4-12). A variety of materials, including metals, glass, polymers, have served as the solid support (13-15) with siliceous materials such as glass and silica perhaps the most common. The biological activity and longevity of surface-bound biomolecules are closely coupled to the geometry and topology of their attachment, rendering detailed understanding of surface conjugation crucial for design of surfaces optimized for applications (e.g. diagnostics, separations). For instance, side reactions with exocyclic base amines or other groups on the DNA may interfere with regiospecificity of attachment as well as activity in hybridization assays (16).

One objective of the present work is to better understand optimal conditions for synthesis of maleimide-activated siliceous supports. Such supports are commonly employed for conjugation of thiolated biomolecules (17-25). Attachment through a terminal thiol of an oligonucleotide, or through the thiol of a cysteine residue on a protein, to the C═C bond of a maleimide provides site-specific immobilization. Previously, the yields and chemical reactivity of the surface over the various synthetic steps involved was difficult to confirm due to challenges in characterizing monolayer or submonolayer films.

High surface area fumed silica powders were used to validate the surface chemistry. Three steps of surface modification were considered: (i) formation of an aminopropyltriethoxysilane (APTES) layer, (ii) reaction of APTES amine groups with heterobifunctional crosslinkers to introduce maleimides, and (iii) immobilization of thiol-terminated DNA strands. Crosslinkers with amine-reactive isocyanates exhibit significantly improved retention of maleimide activity over those employing N-hydroxysuccinimide ester (NHS-ester) as the amine-reactive site. Neither type of crosslinker, however, in general reacts with a 1:1 stoichiometry to surface amines. Rather, isocyanate crosslinkers appear to exceed the amine coverage, while NHS-ester linkers tend to undersaturate it.

Immobilization and hybridization of oligonucleotides on maleimide activated surfaces are also demonstrated. This is the first time that each step of this key methodology for preparing biofunctional surfaces has been examined within a single study, thus affording novel insights into preparation and use of biomolecular films in applications.

### Materials & Methods of Example 1

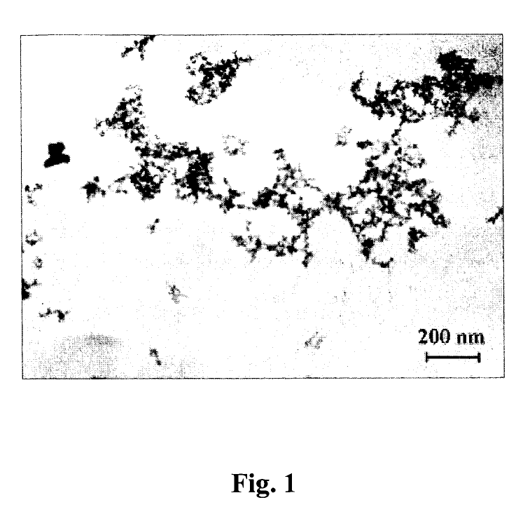

Aerosil® 200 fumed silica was a commercial sample from Degussa-Hüls. This freeflowing powdery material has a manufacturer-specified Brunauer-Emmet-Teller (BET) surface area of 200±25 m²/g. The specific area was independently measured at 199.97 m²/g (Micromeritics Instrument Corp.). The silica consists of aggregates of 12 nm diameter primary, solid silica particles (FIG. 1), with a purity of at least 99.8% amorphous SiO₂.

Aminopropyltriethoxysilane (APTES; 98%) was purchased from Aldrich.

Heterobifunctional crosslinkers p-maleimidophenyl isocyanate (PMPI), maleimidobenzoyl-N-hydroxysuccinimide (MBS), and m-maleimidobenzoyl-Nhydroxysulfosuccinimide (sulfo-MBS) were obtained from Pierce Biotechnology, as was Ellman's reagent (5,5′-dithio-bis-(2-nitrobenzoic acid)). Dithiothreitol (DTT; 99%), L-cysteine (98%), and N-phenylmaleimide (NPM; 97%) were from Aldrich. OliGreen® kit for quantification of single-stranded DNA concentrations was from Molecular Probes Inc. All materials were stored according to provider's instructions and used as received unless indicated otherwise. DNA oligonucleotides, purified by HPLC, were purchased from Qiagen Inc.

The 20mer sequences used in this study are listed in Table 1.1, which also provides the sequence-specific molar absorption coefficients at 260 nm. The sequences were chosen not to have self-complementarity so as to avoid formation of secondary structure (e.g. hairpins).

TABLE 1.1
Oligonucleotide Sequences and Modifications

| | | Extinction | |
|---|---|---|---|
| Abbrevi- | | at 260 nm | |
| ation | Sequence | (M⁻¹cm⁻¹) | Notes |
| PSH | 5′- CGT TGT AAA ACG | 201760 | 3′ thiol-modified |
| | ACG GCC AG-(CH₂)₃- | | probe (sensing |
| | SH-3′ | | strand) |
| | (SEQ. ID NO. 1) | | |
| P | 5′- CGT TGT AAA ACG | 201760 | Unmodified probe |
| | ACG GCC AG-3′ | | |
| | (SEQ. ID NO. 1) | | |
| TC | 5′- CTG GCC GTC GTT | 198770 | Complementary |
| | TTA CAA CG-FL-3′ | | target to PSH, |
| | (SEQ. ID. NO. 2) | | with 3′ fluorescein |
| | | | label |
| TNC | 5′ CTAACT GTT ACC | 198770 | Non-complementary |
| | TCG GTC GG-FL-3′ | | target, with 3′ |
| | (SEQ. ID. NO. 3) | | fluorescein label |

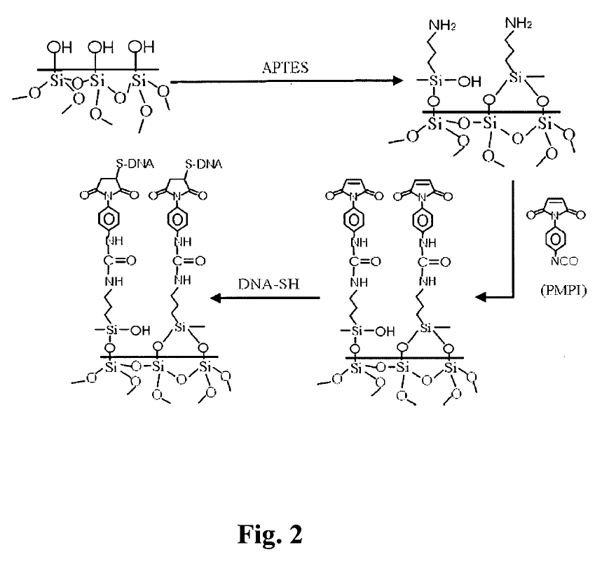

Modification of Fumed Silica. FIG. 2 illustrates the overall scheme for modifying a siliceous surface with DNA, using PMPI crosslinker as an example. The first step, silanization of fumed silica with APTES, proceeded as follows. Typically, 400 mg fumed silica were weighed into a polypropylene tube.

A total of 13 g of 40 mM (1% w/w) solution APTES in anhydrous toluene were added, the tube was sealed, and the mixture shaken for 30 minutes on a vortexer at room temperature. At the end of this period, the powder was centrifuged and the supernatant decanted. The powder was then washed by mechanically dispersing it in fresh solvent, centrifuging, and decanting. A sequence of two toluene washes, one deionized water (Millipore Biocell) wash, and one acetonitrile wash was used. For each wash, 10 ml of fresh solvent was added. Afterward the powder was dried overnight at 100° C. Typical recovery was 70%.

In a second step, the powder was modified with crosslinker (FIG. 2). 20 mg APTES-modified silica was weighed into polypropylene 1.5 ml centrifuge vials and the desired concentration (between 0 and 70 mM) of PMPI or MBS crosslinker in 1.4 ml anhydrous acetonitrile was added. Reaction was carried out for 30 minutes at room temperature in the sealed vial while continually shaken on a vortexer. Acetonitrile was used as solvent due to reports of better suitability than N,N-dimethyl formamide, dimethyl sulfoxide, or aqueous buffers in similar applications (23). The powder was centrifuged and decanted, followed by two 1 ml washes with acetonitrile, one with saline phosphate buffer (PBS: 10 mM sodium phosphate, 1 M NaCl, pH 7), and a third acetonitrile wash. The powder was then dried overnight at 50° C. unless noted otherwise. Both PMPI and MBS linkers are intended to produce a maleimide enriched surface (FIG. 2). The third step, attachment of oligonucleotide, was not carried out on fumed silica, because this would require prohibitively large amounts of DNA. Instead, oligonucleotide attachment and hybridization was performed using flat microscope slides, as described below.

Characterization of Modified Silica. Elemental analysis (Galbraith Laboratories) was used to determine coverage of APTES and PMPI or MBS crosslinker residues on modified silica. APTES coverage was calculated from the increase in carbon content (obtained as a weight percentage) following silanization. The increase in carbon content was converted to surface coverage (APTES/nm²) assuming full hydrolysis of the silane, thus leaving three carbon atoms per APTES residue. This assumption was consistent with near complete lack of absorptions due to methyl groups in infrared spectra, whose presence would have been manifest if significant fraction of silane ethoxy groups remained. Carbon content of unmodified silica, attributed to adventitious surface contamination and to carbon within the silica particles, was subtracted prior to calculating silane coverages. Had it not been subtracted, this background would have been equivalent to about 0.25 APTES/nm². Coverages of crosslinker were similarly calculated from increase in carbon content relative to that from APTES-only powder.

A series of powders were synthesized with varying coverage of APTES and crosslinker and simultaneously analyzed by elemental analysis and transmission infrared spectroscopy (Nicolet Magna 560 IR spectrometer; liquid nitrogen cooled MCT detector). The absolute coverages obtained from elemental analysis of these samples were used to calibrate the infrared absorption measurements. The calibrations enabled subsequent calculation of coverages directly from integration of spectral absorption peaks. The following calibration relations were found:

$$\begin{matrix}APTES \text{:} Molecules \text{/} {nm}^{2} = \frac{1}{1.9} ( \frac{{\int }_{2775}^{3025} A ( v ) ⅆ v}{{\int }_{1751}^{1951} A ( v ) ⅆ v} ) & ( r = 0.995 ) & ( 1 a ) \\ PMPI \text{:} Molecules \text{/} {nm}^{2} = \frac{1}{2.1} ( \frac{{\int }_{1363}^{1430} A ( v ) ⅆ v}{{\int }_{1751}^{1951} A ( v ) ⅆ v} ) & ( r = 0.997 ) & ( 1 b ) \\ MBS \text{:} Molecules \text{/} {nm}^{2} = \frac{1}{0.38} ( \frac{{\int }_{1425}^{1459} A ( v ) ⅆ v}{{\int }_{1751}^{1951} A ( v ) ⅆ v} ) & ( r = 0.994 ) & ( 1 c )\end{matrix}$$

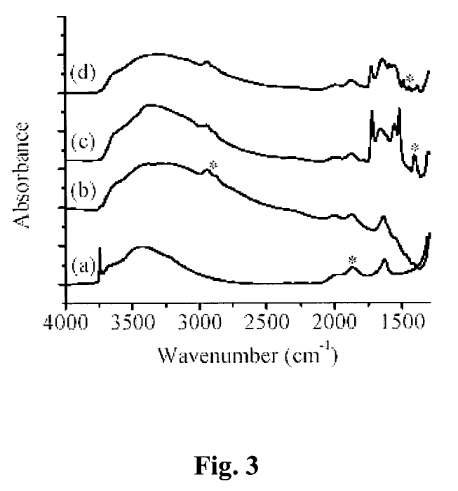

In the above equations, A(v) is absorbance at wavenumber v (cm⁻¹). The prefactors were derived from the elemental analysis calibration. The integration limits span spectral absorption peaks selected to monitor coverage of each species (FIG. 3). The integration from 1751 cm⁻¹ to 1951 cm⁻¹ in the denominator represents overtone vibrations of the silica matrix, and is used as an internal standard to normalize for amount of silica present in the IR beam. This absorption is insensitive to surface modification (26,27). The integrated absorbance in the numerator is proportional to the amount of APTES, PMPI, or MBS present (equations 1a, 1b, or 1c respectively). The particular peaks to integrate were chosen predominantly on criteria of minimal overlap with other spectral features. The ratio of the numerator to denominator is thus proportional to coverage of organic (silane or linker) per amount of powder. A straight line, drawn between the lower and upper limits of each integration, was subtracted as background when integrating the absorbance. Spectral assignments are provided in Table 1.2.

TABLE 1.2
Main Mid-Infrared Spectral Features of Modified Silica (FIG. 3)

| | |
|---|---|
| Mode (cm⁻¹)^{a} | Attribution |

Neat Silica (26, 27)

| | |
|---|---|
| 3744 s | Free silanol O—H stretching |
| ˜3750-3200 | Broad band due to total silanol hydroxyls, adsorbed |
| | H₂O |
| 1980 sh, 1870 m | Overtone structure vibrations of SiO₂ lattice |
| 1630 s | SiO₂ lattice vibrations; bending O—H (molecular |
| | water) |

APTES (28)

| | |
|---|---|
| 3370 w | N—H asymmetric stretch |
| 3303 w | N—H symmetric stretch |
| 2935 m | CH₂ asymmetric stretch |
| 2870 m | CH₂ symmetric stretch |
| 1470 w | CH₂ bending |

PMPI

| | |
|---|---|
| 1776 w | Maleimide symmetric C═O stretch (29) |
| 1717 s | Maleimide asymmetric C═O stretch |
| 1658 s | Amide I (urea) (30) |
| 1550 s | Amide II (urea) (30) |
| 1513 s | Aromatic C—C stretch (29) |
| 1405 s | Maleimide symmetric C—N—C stretch (29) |

MBS

| | |
|---|---|
| 1721 s | Maleimide asymmetric C═O stretch (29) |
| 1636 s | Amide I |
| 1550 s | Amide II |
| 1488 m | Aromatic stretch (31) |
| 1438 m | Aromatic stretch (31) |
| 1388 m | Maleimide symmetric C—N—C stretch (29) |

^{a}s = strong; m = medium; w = weak; sh = shoulder

IR spectra of modified silica were obtained in transmission at 2 cm⁻¹ resolution by sandwiching 3 mg of powder in a cardboard mask cutout between a pair of CaF₂ windows, ensuring uniform distribution of powder across the cross-section of the beam. Scattering losses were at times evident in spectra (FIG. 3, curve b) as a gradually downward sloping baseline toward lower wave numbers, where the longer wavelengths cause greater attenuation due to scattering (FIG. 3).

Following immobilization of PMPI or MBS crosslinker, activity of maleimide groups was determined using Ellman's assay (32,33) according to provider instructions (Pierce Biotechnology). L-cysteine was used to titrate 1 mg aliquots of functionalized powder, and the decrease in bulk concentration of L-cysteine due to reaction with surface maleimides was monitored spectrophotometrically using Ellman's reagent (DTNB: 5,5′-dithio-bis-(2-nitrobenzoic acid)). DTNB reacts with the sulfhydryl group of free L-cysteine molecules to yield a mixed disulfide and the colored species 2-nitro-5-thiobenzoic acid, which provides the spectrophotometric signal. Assuming a 1:1 stoichiometry of reaction between L-cysteine and surface maleimides, the surface coverage of active and accessible maleimide groups was estimated. APTES modified silica served as control.

Immobilization of Oligonucleotides on Glass Slides. Glass slides (Fisherfinest Premium Microscope Slides) were sonicated 10 minutes each in chloroform, ethanol, water, and dried. The slides were modified with APTES and PMPI crosslinker prior to DNA immobilization. Modification with APTES was performed under 2.8 mM solution in anhydrous toluene for 30 minutes at room temperature while stirring. Slides were washed twice with toluene, once with deonized water, once with acetonitrile, followed by drying overnight at 100° C. Duration of each wash was 10 minutes. Next, APTES-coated slides were exposed to 0.5 mg/ml PMPI in anhydrous acetonitrile for 2 hrs at room temperature with gentle stirring. After attachment of PMPI, slides were washed once with fresh acetonitrile and once with PBS (10 minutes per wash). Disulfide terminated PSH oligonucleotides (Table 1) were cleaved with DTT in PBS (200 fold excess DTT over disulfide; 1 hr) to liberate the thiol groups and purified on size exclusion PD-10 columns (Amersham Biosciences). Solutions of freshly prepared PSH oligonucleotides were used immediately to functionalize PMPI-derivatized slides, using concentrations of 0.1 μM or 1 μM oligonucleotide in PBS (2 hrs; room temperature). For this step a 1.5 ml chamber, closed to the ambient environment, was created by sandwiching a silicone O-ring between two glass slides that were identically activated with PMPI. DNA solutions were introduced to the chamber via a syringe and needle inserted through the O-ring, with the O-ring thus doubling as a septum. The unmodified P oligonucleotides were attached in an identical fashion except for the omission of the DTT cleavage step.

Coverage of immobilized oligonucleotides was determined from decrease in bulk concentration of DNA, determined by comparing concentrations before and post attachment. Sensitive measurement of DNA concentration was performed using OliGreen® fluorescent stain for single-stranded DNA from Molecular Probes according to manufacturer's instructions, on an SLM 8000 fluorometer. The fluorescence signals were calibrated from serial dilutions of more concentrated solutions whose DNA concentrations were determined by absorption measurements at 260 nm, using the molar absorption coefficients in Table 1.

Hybridizations. Surfaces derivatized with PSH oligonucleotides were exposed to 0.1 μM solutions of fully complementary TC strands and, as a control for sequence specificity of hybridization, to non-complementary TNC target sequences in PBS. Hybridizations were allowed to proceed for 30 hrs at room temperature. These long hybridization times, compared to more typical durations of several hours (16-20,22), were intended to allow sufficient time for target sequences to hybridize to near equilibrium even when the probe surfaces were highly crowded. As discussed below, PSH probe densities of ˜2×10¹³ strands/cm² were achieved; under comparably dense coverages, durations of up to 16 hrs have been shown to increase hybridization yields (34). It should be noted, however, that hybridization kinetics were not followed in the present study and therefore achievement of equilibrium hybridization was not confirmed.

The TC and TNC strands were labeled with fluorescein so as to allow quantification of hybridization extents from decrease in fluorescence of bulk solutions. As hybridization yields were determined from changes in fluorescence of bulk solutions, washing or other post-processing of the surface, which could perturb the extent of hybridization, did not affect the measured values. Additional, albeit qualitative, confirmation of hybridization was obtained by laser scanning confocal microscopy (Olympus IX-70 microscope equipped with an Ar/Kr laser) carried out directly on hybridized slides. For these measurements hybridized slides were washed twice with PBS (2 minutes per wash) and dried with compressed nitrogen prior to scanning. Fluorescence confocal microscopy proved not suitable for quantitative work due to fluorescence quenching at higher extents of hybridization (35).

### Results and Discussion of Example 1

Attachment of APTES. The selection of APTES was motivated by unique properties of aminosilane reagents. It was desirable to employ anhydrous conditions to minimize hydrolysis and oligomerization of the silane in bulk solution to improve reproducibility of the resultant surface monolayer, with the ideal outcome consisting of a layer of molecules each of which is covalently bound to the silica support via one or more siloxane bridges (FIG. 2). Direct formation of siloxane bonds between alkoxysilanes and surface silanols under anhydrous conditions requires an amine catalyst (36). Since aminosilanes such as APTES contain the catalyzing amine functionality in the same molecule, a significant fraction of adsorbed aminosilanes attaches covalently to the solid support even under ambient temperature and anhydrous conditions (37,38). In addition to the desired formation of siloxane bonds, APTES interacts with surface silanols through the amine terminus via hydrogen bonding and ionic interactions (39-41). Thus, bound APTES molecules are expected to adopt a distribution of conformations on the silica surface.

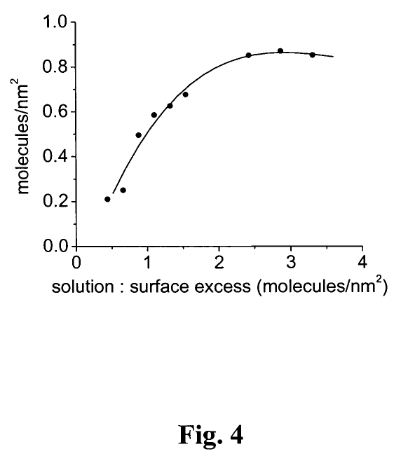

FIG. 4 shows APTES coverage (molecules/nm²) on Aerosil® 200 silica after a 30 minute exposure to solutions in anhydrous toluene, followed by washing and drying. The x-axis “solution: surface excess” represents how many silane molecules were initially added to bulk solution per nm2 of surface available for attachment. Short reaction times were employed to realize submonolayer coverages of APTES (42). For the employed protocol, coverage approached a plateau close to 0.9 APTES molecules/nm² when bulk to surface excess exceeded 2.5 molecules/nm², corresponding to a concentration of 0.4% w/w. Most literature studies of APTES reaction with silica under anhydrous conditions report chemical (irreversible) loadings of 2 molecules/nm² or greater (39,40,43), well above 0.9 molecules/nm² of this study. However, Aerosil® 200 has fewer surface silanols than the fully hydroxylated silica typically used, about 2.8 silanols/nm² (44) compared to 4.6 silanols/nm² (45). For partially dehydroxylated silica surfaces with comparable silanol densities to Aerosil® 200, Vrancken et al reported a coverage of about 1.7 APTES/nm² after a 2 hr deposition from 1% toluene solutions and curing for 20 hrs under vacuum at 423° K (46). In the present Example, only a 30 minute deposition time was used and the samples were not cured but washed immediately, so that only those silane molecules that react covalently during the relatively short deposition step are expected to remain. The lower APTES loadings are attributed to the above differences in preparation protocol. The studies described below employed silica with the plateau, submonolayer coverage of ˜0.8 to 0.9 APTES molecules/nm².

Linker Immobilization and Activity. The heterobifunctional crosslinker PMPI was developed for preparing protein conjugates (47). The isocyanate moiety in this linker (FIG. 2) is highly reactive toward alcohol or amine groups, forming stable carbamate or urea bonds respectively, whereas the linker's maleimide moiety reacts with thiols to create thioether links. The steric accessibility of the isocyanate group also renders it ideal for reacting in the crowded environment of a surface, where saturation of reactive surface groups is desired.

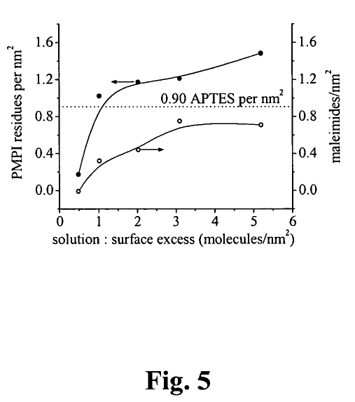

As the isocyanate moiety is unstable in water, reaction of PMPI with APTES-modified silica was carried out in anhydrous acetonitrile. In infrared spectra, PMPI attachment was confirmed by disappearance of APTES amine stretches at 3303 and 3370 cm⁻¹, consistent with the amines reacting with PMPI to form urea linkages (FIG. 2). Moreover, several strongly absorbing modes associated with PMPI appeared in the 1400-1800 cm⁻¹ region (FIG. 3; Table 2). FIG. 5 plots surface coverage of PMPI and accompanying maleimide activity as a function of bulk concentration of linker applied to the APTES-modified silica. The x-axis is defined as in FIG. 4, with a unit of one corresponding to a concentration of 13 mM PMPI in acetonitrile; thus, the concentration range investigated was 0.62 mM to 67 mM. PMPI coverage was determined from infrared absorption spectra via equation 1b, while maleimide activity was obtained using Ellman's analysis. A dotted line is drawn to indicate surface coverage of APTES molecules on the silica, which was 0.90 molecules/nm².

Surprisingly, FIG. 5 shows the PMPI coverage (filled circles) to rise fairly quickly to surpass that of APTES. It appears that achievement of a 1:1 stoichiometry between PMPI molecules and APTES silanes is not limiting; in other words, more linker molecules attach than there are available amines. On the other hand, analysis via Ellman's assay showed the active maleimide coverage to approach, but not to exceed, that of APTES (FIG. 5). Therefore, there are fewer active maleimides than PMPI residues, and a fraction of the maleimides are either inaccessible to L-cysteine or have become inactive.

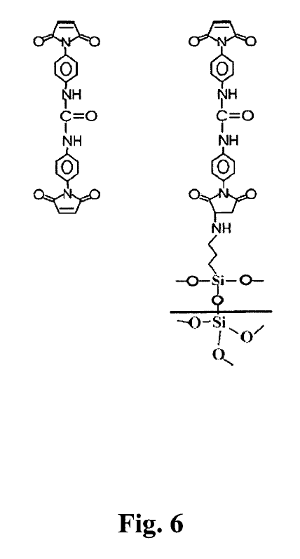

These observations may be interpreted as follows. Inevitably, trace water will be present in the reaction mixture, with primary source presumably physisorbed moisture associated with the high surface area of the fumed silica. The isocyanate group of PMPI readily reacts with water to form an unstable carbamic acid which decomposes to evolve CO₂ and to leave an amine group in place of the isocyanate (48). The generated amine reacts with a second PMPI molecule to form the bismaleimide compound N,N′-bis(p-maleimidophenyl)urea (47), which attaches to surface amines via Michael addition to one of the maleimide termini (FIG. 6). Maleimide-amine additions are well known and used in a variety of applications, e.g. polymer resins (49). In the case of the bismaleimide, two PMPI residues are introduced per surface silane. Other possibilities include reaction of hydrolyzed, amine-containing PMPI molecules with PMPI maleimides already on the surface, opening a second avenue for vertical stacking of PMPI residues. Regardless of the stacking mechanism, however, the number of active maleimides per area cannot exceed the number of APTES molecules available, in agreement with the data of FIG. 5. To summarize, the difference between coverage of PMPI residues and active maleimides is believed to reflect a distribution of reaction products on the surface, encompassing both single and multiply stacked PMPI adducts.

The occurrence of Michael addition between APTES derivatized silica and maleimide groups was independently confirmed using N-phenylmaleimide (NPM). NPM is identical to PMPI except for replacement of the isocyanate group by hydrogen. A two hour reaction with 33 mM NPM in acetonitrile at room temperature resulted in 0.34 NPM molecules/nm² by elemental analysis but only 0.05 active maleimides/nm², a barely detectable activity. These results strongly indicate that NPM attached via its maleimide moiety.

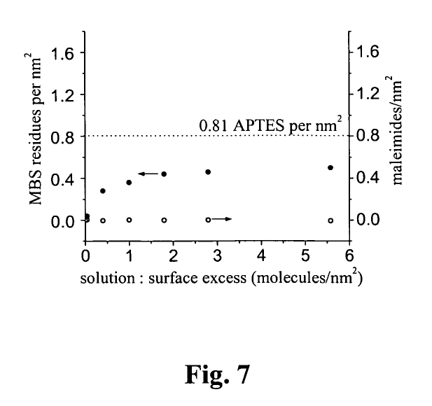

Heterobifunctional linkers toward amine and thiol compounds most often rely on NHS-ester as the amine-reactive site (50). As a comparison to PMPI, the NHS-ester linker MBS was investigated. In infrared spectra, attachment of MBS was identified by disappearance of APTES amine N—H stretches and appearance of modes attributed to MBS and the formation of an amide linkage to surface amines (Table 2, FIG. 3). FIG. 7 plots MBS coverage and maleimide activity as a function of bulk concentration of linker used in the derivatization. The highest concentration, corresponding to a solution to surface excess of 5.58 molecules/nm², represents 53 mM. The observed trends are strikingly different from those for PMPI (FIG. 5). The linker coverage reaches at most 60% that of the silane. Therefore, saturation of the aminosilane layer with linker does not occur. Second, the activity of maleimide groups is very low. These results indicate a failure of the MBS linker to generate significant enrichment of maleimide groups on the APTES surface.

The evident lack of active MBS maleimides may be understood as follows. If surface amines are not consumed quickly enough during attachment of MBS molecules, then an immobilized (via the NHS ester terminus) MBS molecule will be surrounded by remnant amines. Given sufficient time, the maleimide of the linker becomes deactivated by reaction with one of the neighboring amines. This sequence of events would leave MBS linkers attached via both ends, the desired amide bond and the undesired Michael addition through the maleimide. In addition to explaining the low remnant maleimide activity (FIG. 7), the above mechanism rationalizes why MBS coverage does not approach that of APTES since two surface amines are consumed per doubly-bonded linker. In contrast, the enhanced reactivity of the PMPI isocyanate group, as opposed to the NHS-ester of MBS, evidently allows the PMPI linker to more quickly and completely react with surface amines.

In addition to polar organic solvents such as acetonitrile, surface immobilization of heterobifunctional crosslinkers bearing NHS-ester and maleimide sites (e.g. sulfo-MBS) is often performed under aqueous conditions. In an aqueous buffer, protonation and hence reactivity of the APTES amines would be subject to pH. A potential complication arises from the high concentration of amines at the solid support, which presumably renders the local conditions more basic than those in bulk buffer. Basic pH speeds hydrolysis of the NHS-ester or maleimide moieties of the linker, as well as influence the reactivity of amines with these groups. Experiments were performed with the water soluble version of MBS, sulfo-MBS, to determine yields of active maleimides under typical conditions.

The sulfo-MBS crosslinker was reacted with APTES functionalized silica APTES/nm²) from an aqueous buffer (20 mM sodium phosphate, 0.15 M NaCl, pH 7.0). As with MBS, the reactive sites on sulfo-MBS are an NHS-ester and a maleimide. Very low (less than 0.06 active maleimides/nm²) activity was observed when sulfo-MBS was immobilized from 5 mM and 20 mM concentrations, corresponding to a solution to surface excess of 0.5 and 2 molecules/nm². However, significant activity of 0.3 maleimides/nm² was realized when the silica was reacted with 40 mM linker solution (solution to surface excess of 4 molecules/nm²). Thus, by raising the concentration of linker, it was possible to saturate the surface sufficiently rapidly to retain a fraction of active maleimide groups. Recovery of maleimide activity at higher linker concentrations is also expected with MBS attachment from acetonitrile, though this was not realized under investigated conditions (FIG. 7). Qualitatively, in terms of effectiveness for introducing maleimide groups on aminosilanized silica, our results suggest using PMPI in acetonitrile, followed by sulfo-MBS in pH 7 buffer, with MBS in acetonitrile producing the poorest yields.

Assays for maleimide activity were carried out after drying of the silica powder, postponing analysis by a day. In order to determine maleimide activity immediately after preparation, PMPI and MBS modified silica was also characterized while still wet with acetonitrile solvent. Therefore, a small amount (approx. 0.7% v/v) of acetonitrile was present during the titration with L-cysteine. Linker immobilization was carried out from 50 mM solutions in acetonitrile for 1 hr in sealed 1.5 ml plastic vials, with the PMPI and MBS samples prepared and characterized side by side under identical conditions.

Transmission IR revealed the PMPI sample to possess 0.8 APTES per nm² and 1.1 PMPI molecules per nm². With these coverages, there should be very few unreacted amines left on the surface. The density of active maleimides immediately post attachment was found to be 0.75 per nm², while after 23 hours the density decreased to 0.70 per nm². Incidentally, this slight decrease indicates that deactivation of maleimides by ambient (e.g. thiol-containing) contaminants during laboratory storage of modified powders, if present, was minimal. The MBS sample, with 0.9 APTES per nm² and 0.45 MBS per nm², had 0.1 active maleimides per nm² immediately after attachment which decreased to undetectable levels (less than 0.05 per nm²) after 5 hours. Therefore, virtually all MBS maleimides become deactivated during the relatively brief derivatization of the aminosilanized surface, as opposed to during subsequent drying or other processing of the powder.

Control experiments were carried out in which silica without APTES was exposed to linker solutions (24 mM PMPI or 30 mM MBS in acetonitrile), followed by standard washing and drying of the powder. IR measurements showed no evidence of linker attachment, and no maleimide activity was detected when the powders were titrated with L-cysteine. These results indicate that neither PMPI nor MBS formed stable bonds with surface silanols; therefore, the APTES monolayer allows surface conjugation of crosslinkers.

DNA Immobilization and Hybridization. Demonstration of PMPI-modified surfaces for immobilization of DNA oligonucleotides and their subsequent hybridization was performed on planar microscope slides. Glass slides were used because the high surface area of fumed silica would require prohibitive quantities of oligonucleotide. While silanization and subsequent modification of silica and glass are expected to be qualitatively similar, glass contains other metal oxides in addition to the major SiO₂ component and hence quantitative differences (e.g. in APTES and linker coverage) between silica and glass may be expected. Attachment of the 20mer oligonucleotide PSH was performed from 1 μM or 0.1 μM solutions in PBS for 15 hours at room temperature. Approximately 1.5 ml of solution was injected into a chamber created by a silicone O-ring between two PMPI-activated glass slides of known area. Because oligonucleotide immobilization was quantified by fluorometric quantification of decrease in strand concentration in bulk solution, potential loss of accuracy could result from adsorption of oligonucleotides to the internal O-ring surfaces. Separate control experiments confirmed such losses to be less than 5%. As an additional control to check that attachment occurred site specifically via the thiol moiety the P oligonucleotide, with the same sequence as PSH but lacking the 3′ thiol modification (Table 1), was immobilized following the same protocol.

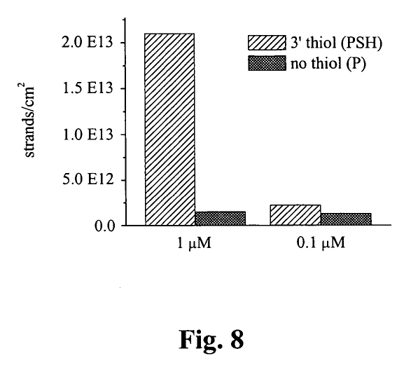

FIG. 8 displays the results of these experiments. For the PSH oligonucleotide, the higher 1 μM concentration yielded a dense DNA monolayer with 2.1×10¹³ strands/cm². For the 0.1 μM reaction, the final coverage was 2.2×10¹² strands/cm². In addition to bulk concentration of oligonucleotide, the realized coverages depend on the extent of generation by DTT of reactive thiol groups on the as received oligonucleotides as well as any subsequent thiol oxidation that may take place prior to immobilization (17,51). Although these other factors were not analyzed in detail, the results demonstrate that a range of surface coverage up to very dense layers is attainable.

While inclusion of a thiol endgroup enhanced immobilization, FIG. 8 also shows that significant, ˜1.2×10¹² strands/cm², coverage was reached even with the P oligonucleotide which lacked a thiol. Therefore, unmodified DNA oligonucleotides are capable of either physically adsorbing or covalently binding to the surface via a site other than a terminal thiol (e.g. a base amine or a 3′ hydroxyl). Chrisey et al. reported that physically adsorbed oligonucleotides on maleimide-derivatized aminosilane surfaces could be desorbed by immersion in high ionic strength solutions (52). In the present study, oligonucleotides were immobilized from 1 M NaCl buffer so that physical adsorption would be expected to be similarly suppressed, possibly indicating that direct covalent attachment is the more likely explanation for the observed coverage of P oligonucleotide.

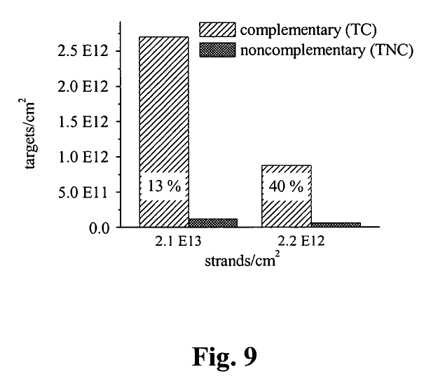

Hybridization of PSH oligonucleotide layers to complementary TC and noncomplementary TNC target strands was performed simultaneously using different regions of the same slide, as defined by separate silicone O-rings. As FIG. 9 shows, hybridization was sequence specific with signals from non-complementary targets less than 10% of those from the fully complementary sequence. On the high coverage surface with 2.1×10¹³ strands/cm², only 13% of bound oligonucleotides underwent hybridization with the complementary TC targets. The low yield likely reflects steric crowding as the theoretical jamming coverage for double-stranded DNA is about 3×10¹³ per cm², close to the density of the immobilized PSH strands. Other reports have noted decreases in hybridization yields at coverages exceeding ˜5×10¹² strands/cm² (53-56).

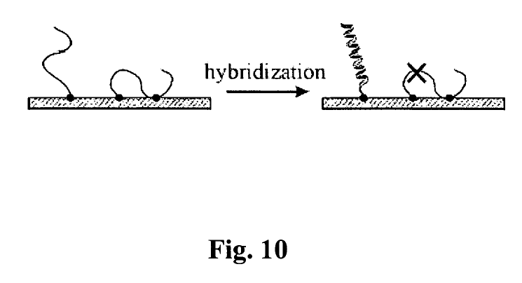

In contrast, other studies (5,54) indicate that close to 100% hybridization yields should be achievable at the lower coverage of 2.2×10¹² strands/cm², presuming each immobilized PSH strand is active and accessible. Observation of hybridization yields of 40% therefore suggests that 60% of the surface tethered strands were inactive. One source of strand deactivation is depicted in FIG. 10. If, as illustrated, a strand becomes bound at more than one point along its backbone then the end-to-end distance of the segment between the immobilized points is constrained. If this distance deviates significantly from the length required to accommodate a rigid, hybridized duplex then, as discussed by Bünemann (57), binding of a target TC strand may be prevented. Here, 60% of inactive strands represents a coverage of 1.3×10¹² strands/cm². This value is comparable to the 1.2×10¹² strands/cm² coverage obtained with nonthiolated P oligonucleotides (FIG. 8). In other words, if inactivity toward hybridization is attributed to formation of surface crosslinks via multiple sites (FIG. 10), the surface density of crosslinks required is consistent with that needed to explain immobilization of the P oligonucleotides.

### Conclusions of Example 1

This report highlighted preparation of maleimide-activated siliceous supports for immobilization of biomolecules. Two different crosslinker chemistries were compared in their ability to activate aminosilanized silica with maleimides, and application to immobilization and hybridization of DNA oligonucleotides was demonstrated. amine groups were first introduced by silylation of the surface with aminopropyltriethoxysilane (APTES). It was found that PMPI, a crosslinker bearing sterically accessible, highly reactive isocyanate groups was significantly more successful at saturating surface (APTES) amines from a polar organic solvent (acetonitrile) than MBS, which uses NHS-esters as the amine-reactive site. Perhaps surprisingly, coverage of PMPI residues could exceed that of the base APTES layer, suggesting formation of dimer and possibly higher adducts of PMPI facilitated by trace water. In contrast, MBS systematically undersaturated the coverage of surface amines, and MBS modified silica exhibited low levels of maleimide activity. These observations were explained within the context of a Michael-addition side reaction between APTES amines and crosslinker maleimides, leaving MBS linkers attached via their maleimide as well as NHS-ester sites. Thiol-terminated, single-stranded oligonucleotides immobilized on PMPI-activated surfaces were shown capable of binding complementary strands from solution at yields of up to 40%. Further optimization of oligonucleotide conjugation to maleimide-decorated surfaces may include prevention of attachment through sites other than the terminal oligonucleotide thiol. The results of this study advance capability to precisely tailor siliceous surfaces with biological molecules, and should help advance applications as well as fundamental investigations of biomolecules at interfaces.

### REFERENCES OF EXAMPLE 1

(1) Southern, E. M. Trends in Genetics 1996, 12, 110.
(2) Lockhart, D. J.; Winzeler, E. A. Nature 2000, 405, 827.
(3) Staudt, L. M.; Brown, P. O. Annu. Rev. Immunol. 2000, 18, 829.
(4) Steel, A. B.; Levicky, R. L.; Herne, T. M.; Tarlov, M. J. Biophys. J. 2000, 79, 975.
(5) Levicky, R.; Herne, T. M.; Tarlov, M. J.; Satija, S. K. J. Am. Chem. Soc. 1998, 120, 9787.
(6) Georgiadis, R.; Peterlinz, K. P.; Peterson, A. W. J. Am. Chem. Soc. 2000, 122, 3166.
(7) Heaton, R. J.; Peterson, A. W.; Georgiadis, R. M. Proc. Natl. Acad. Sci. U.S.A. 2001, 98, 3701.
(8) Chan, V.; McKenzie, S. E.; Surrey, S.; Fortina, P.; Graves, D. J. J. Colloid Interface Sci. 1998, 203, 197.
(9) Su, H.-J.; Surrey, S.; McKenzie, S. E.; Fortina, P.; Graves, D. J. Electrophoresis 2002, 23, 1551.
(10) Watterson, J. H.; Piunno, P. A. E.; Wust, C. C.; Krull, U. J. Langmuir 2000, 16, 4984.
(11) Piunno, P. A. E.; Watterson, J.; Wust, C. C.; Krull, U. J. Anal. Chim. Acta 1999, 400, 73.
(12) Walker, H. W.; Grant, S. B. Langmuir 1995, 11, 3772.
(13) Beaucage, S. L. Cur. Medicinal Chem. 2001, 8, 1213.
(14) Henke, L.; Krull, U. J. Can. J. Anal. Sci. Spectrosc. 1999, 44, 61.
(15) Tarlov, M. J.; Steel, A. B. In Biomolecular Films: Design, Function, and Applications; Rusling, J. F., Ed.; Marcel Dekker: New York, 2003, pp 545-608.
(16) Yang, M. S.; Kong, R. Y. C.; Kazmi, N.; Leung, A. K. C. Chem. Lett. 1998, 257.
(17) Chrisey, L. A.; Lee, G. U.; O'Ferrall, C. E. Nucl. Acids Res. 1996, 24, 3031.
(18) Adessi, C.; Matton, G.; Ayala, G.; Turcatti, G.; Mermod, J.-J.; Mayer, P.; Kawashima, E. Nucl. Acids Res. 2000, 28, e87.
(19) Okamoto, T.; Suzuki, T.; Yamamoto, N. Nat. Biotechnol. 2000, 18, 438.
(20) Cavic, B. A.; McGovern, M. E.; Nisman, R.; Thompson, M. Analyst 2001, 126, 485.
(21) Andreadis, J. D.; Chrisey, L. A. Nucl. Acids Res. 2000, 28, e5.
(22) Oh, S. J.; Cho, S. J.; Kim, C. O.; Park, J. W. Langmuir 2002, 18, 1764.
(23) Xiao, S.-J.; Textor, M.; Spencer, N. D. Langmuir 1998, 14, 5507.
(24) MacBeath, G.; Koehler, A. N.; Schreiber, S. L. J. Am. Chem. Soc. 1999, 121, 7967.
(25) Hong, H. G.; Bohn, P. W.; Sligar, S. G. Anal. Chem. 1993, 65, 1635.
(26) Gorski, D.; Klemm, E.; Fink, P.; Horhold, H.-H. J. Coll. Int. Sci. 1988, 126, 445.
(27) Vansant, E. F.; Van Der Voort, P.; Vrancken, K. C. Characterization and Chemical Modification of the Silica Surface; Elsevier: New York, 1995; p. 65.
(28) Chiang, C. H.; Ishida, H.; Koenig, J. L. J. Colloid Interface Sci. 1980, 74, 396.
(29) Parker, S. F.; Mason, S. M.; Williams, K. P. J. Spectrochim. Acta 1990, 46A, 315.
(30) de Zea Bermudez, V.; Carlos, L. D.; Alcacer, L. Chem. Mater. 1999, 11, 569.
(31) Lin-Vien, D.; Colthup, N. B.; Fateley, W. G.; Grasselli, J. G. The Handbook of Infrared and Raman Characteristic Frequencies of Organic Molecules; Academic Press: New York, 1991; p. 281.
(32) Ellman, G. L. Arch. Biochem. Biophys. 1959, 82, 70.
(33) Riddles, P. W.; Blakeley, R. L.; Zerner, B. Anal. Biochem. 1979, 94, 75.
(34) Maskos, U.; Southern, E. M. Nucleic Acids Res. 1992, 20, 1679.
(35) McGall, G. H.; Barone, A. D.; Diggelmann, M.; Fodor, S. P. A.; Gentalen, E.; Ngo, N. J. Am. Chem. Soc. 1997, 119, 5081.
(36) Blitz, J.; Murthy, R. S. S.; Leyden, D. E. J. Am. Chem. Soc. 1987, 109, 7141.
(37) Vansant, E. F.; Van Der Voort, P.; Vrancken, K. C. Characterization and Chemical Modification of the Silica Surface; Elsevier: New York, 1995; p. 243.
(38) White, L. D.; Tripp, C. P. J. Colloid Interface Sci. 2000, 227, 237.
(39) Caravajal, G. S.; Leyden, D. E.; Quinting, G. R.; Maciel, G. E. Anal. Chem. 1988, 60, 1776.
(40) Kallury, K. M. R.; Macdonald, P. M.; Thompson, M. Langmuir 1994, 10, 492.
(41) Vandenberg, E. T.; Bertilsson, L.; Liedberg, B.; Uvdal, K.; Erlandsson, R.; Elwing, H.; Lundstrom, I. J. Coll. Interfac. Sci. 1991, 147, 103.
(42) Moon, J. H.; Kim, J. H.; Kim, K. J.; Kang, T. H.; Kim, B.; Kim, C. H.; Hahn, J. H.; Park, J. W. Langmuir 1997, 13, 4305.
(43) Trens, P.; Denoyel, R. Langmuir 1996, 12, 2781.
(44) Mueller, R.; Kammler, H. K.; Wegner, K.; Pratsinis, S. E. Langmuir 2003, 19, 160.
(45) Zhuravlev, L. T. Colloid Surf. A-Physicochem. Eng. Asp. 2000, 173, 1.
(46) Vrancken, K. C.; Van Der Voort, P.; Possemiers, K.; Vansant, E. F. J. Colloid Interface Sci. 1995, 174, 86.
(47) Annunziato, M. E.; Patel, U.S.; Ranade, M.; Palumbo, P. S. Bioconjugate Chem. 1993, 4, 212.
(48) Smith, M. B.; March, J. March's Advanced Organic Chemistry; 5th ed.; John Wiley & Sons, Inc.: New York, 2001; p. 1178.
(49) Crivello, J. V. J. Polym. Sci. Pol. Chem. 1973, 11, 1185.
(50) Brinkley, M. Bioconjugate Chem. 1992, 3, 2.
(51) O'Donnell, M. J.; Tang, K.; Koster, H.; Smith, C. L.; Cantor, C. R. Anal. Chem. 1997, 69, 2438.
(52) Chrisey, L. A.; Roberts, P. M.; Benezra, V. I.; Dressick, W. J.; Dulcey, C. S.; Calvert, J. M. In Materials Research Society Symposium Proceedings; Alper, M., Bayley, H., Kaplan, D., Navia, M., Eds.; Materials Research Society: Pittsburgh, Pa., 1994; Vol. 330, pp 179.
(53) Walsh, M. K.; Wang, X.; Weimer, B. C. J. Biochem. Biophys. Methods 2001, 47, 221.
(54) Steel, A. B.; Herne, T. M.; Tarlov, M. J. Anal. Chem. 1998, 70, 4670.
(55) Podyminogin, M. A.; Lukhtanov, E. A.; Reed, M. W. Nucl. Acids Res. 2001, 29, 5090.
(56) Beattie, W. G.; Meng, L.; Turner, S. L.; Varma, R. S.; Dao, D. D.; Beattie, K. L. Molec. Biotechnol. 1995, 4, 213.
(57) Bunemann, H. Nucl. Acids Res. 1982, 10, 7181.

### EXAMPLE 2

### Materials and Experimental Methods

Introduction. Development of chemistries for attachment of polynucleic acids to siliceous surfaces such as glass and silica, and of protocols for characterization of the modified surfaces was investigated. The immobilization chemistries, described in Example 3, support subsequent physical studies of DNA properties and function at interfaces as well as development of devices such as DNA chips and microarrays with applications in functional genomics, pharmacogenomics, pathogen identification, and other areas.¹⁻³ In this example, the materials, methods, and protocols employed are introduced.

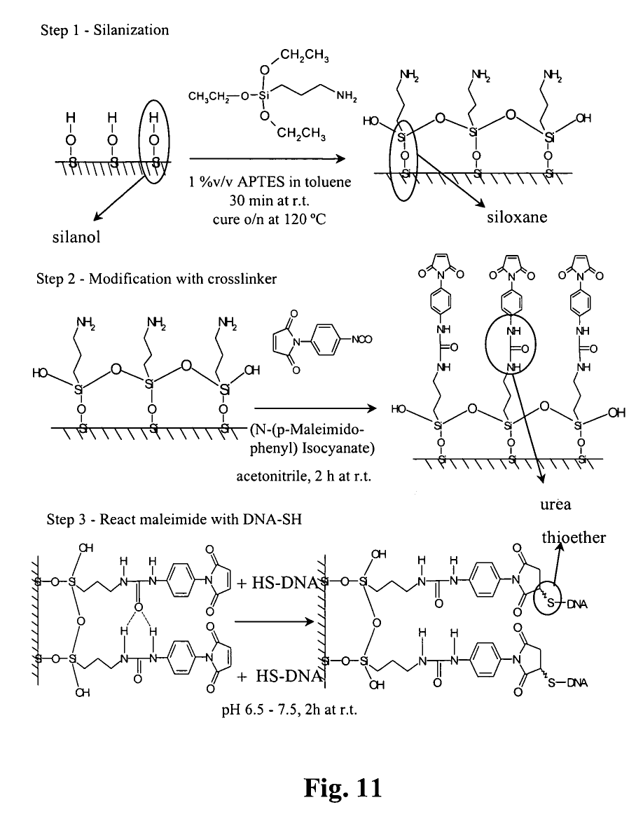

While details of the immobilization chemistry are postponed to Example 3, here a specific example is provided. The investigated methods for attaching DNA to siliceous surfaces consist of three principal steps. In the first step, surface silanol groups are modified with a silane layer to introduce amine groups to the surface (FIG. 11). In the second step, a crosslinking reagent is used that reacts with the amine and enriches the surface in maleimide moieties. In the third and final step, sulfhydryl (thiol) modified DNA oligonucleotides are attached via thioether bonds to the maleimides. The entire scheme is depicted in FIG. 11, using as examples 3-aminopropyltriethoxysilane (APTES) as the silane and N-(p-maleimidophenyl) isocyanate (PMPI) as the crosslinker. The silanol surfaces were either fumed amorphous silica (amorphous SiO₂) or glass (or fused silica) microscope slides. The thiolated DNA used was commercially purchased, and was typically 20 nucleotides long (i.e. a “20mer”). In the following sections these materials are examined in greater detail.

Materials.

Surfaces. The fumed silica, amorphous SiO₂, supports was Aerosil® 200 powder from Degussa. FIG. 1 depicts a transmission electron microscopy (TEM) image of Aerosil® 200 obtained on a JEOL JEM-100C TEM. Aerosil® 200 is a hydrophilic fumed silica. This material is a fluffy white powder which has an extremely low bulk density in the range of about 30 g/L of powder, and specific surface area of about 200±25 m²/g. The silica particles have the density of amorphous SiO₂, about 2200 g/L.⁴ At the submicron scale, as shown in FIG. 1, fumed silica consists of aggregated primary particles, which for Aerosil® 200 are about 12 nm in diameter. These particles, firmly attached or partially fused, aggregate into large, branched, fractal like structures. On the surface of the silica there are many silanol, or Si—OH, groups. Silylation of these silanol groups with organosilicon reagents is the usual route to surface modification.

Aerosil® 200 was donated by Degussa (www.degussa.com). It is very pure silica, with SiO₂ content greater than 99.8%. The Aerosil® powders are named according to their approximate specific surface area, with the 200 signifying 200 m²/g. Among available fumed silicas, Aerosil® 200 has an intermediate specific surface area. Importantly, it may be compacted and packed quite well, making for less light scattering during measurements such as infrared absorption spectroscopy (see below).

On occasion, fused silica slides were used, which were purchased from Structure Probe Inc. (SPI) (www.2spi.com). These slides are about 1 mm thick, and 1″×3″ in area; that is, typical size of a microscope slide. The quartz sand used for the production of the SPI slides is considered “high purity,” and is of the same quality as used in the electronics industry which has the highest specifications for quality. However, when planar supports were needed, glass microscope slides were used (e.g. Fisherfinest Premium Microscope Slides), which have a lower content of SiO₂ (about 60%) with the rest various metal oxides.

The reason for using both high surface area supports, such as fumed Aerosil® 200 silica, and planar supports such as microscope slides were as follows. Because it is difficult to detect an organic monolayer on a planar surface with common analytical techniques, including Fourier Transform Infrared (FTIR) spectroscopy and elemental analysis, high surface area supports were used to increase the surface to sample size ratio. However, due to prohibitive costs of DNA oligonucleotides, it was not affordable to functionalize such high surface samples with nucleic acids. Therefore, for characterization of the third step of immobilization in which DNA is attached (FIG. 11), planar microscope slides were used.

Silanes. Two types of aminosilanes were used in the first step of SiO₂ surface modification. They are (3-aminopropyl)dimethylethoxysilane (APDMES) and (3-aminopropyl)triethoxysilane (APTES), both obtained from Gelest Inc. The structure of APDMES is 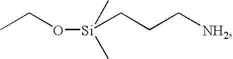
and its molecular weight is 161.32 g/mol. The product from Gelest, Inc. is 95% pure, and is a clear liquid at room temperature. It has a boiling point of 78° C. The chemical structure of APTES is 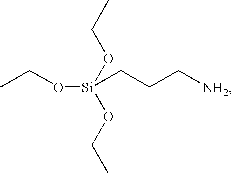
and its molecular weight is 221.4 g/mol. The product from Sigma is 98% pure and, like APDMES, APTES is a clear liquid. It has a boiling point of 122° C.

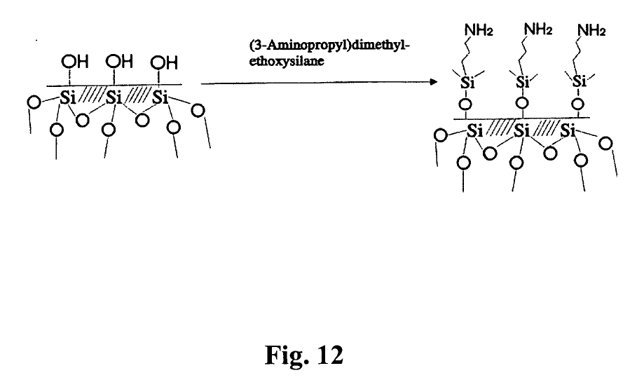

APDMES possesses a single ethoxy leaving group through which, if an APDMES molecule attaches to the surface, an exclusive siloxane bridge to a surface silanol is made (FIG. 12). In contrast, the three ethoxy leaving groups of APTES allow for the simultaneous possibility of attachment to the surface as well as formation of siloxane links between neighboring APTES molecules, with the result that APTES may produce multilayers. Multilayers may occur since the silicon atoms in APTES have multiple ethoxy leaving groups, allowing the silicon atom to form multiple new bonds.⁶ Thus, to assure that only a monolayer forms, APDMES is the better choice.

However, as shown in Example 3, APDMES monolayers were labile. In particular, repeated washings of the APDMES surface with water lead to a steady, significant attrition of the silane coverage. In order to improve stability of the silane films, APTES was tested. Although APTES may produce multilayers, submonolayer coverages are readily achieved by controlling the reaction time. In addition, APTES monolayers are more stable, because multiple siloxane bonds to surface silanols may form, and because nearby APTES molecules may crosslink to each other, which further enhances monolayer stability.

APDMES and APTES were typically attached to the silica surface from anhydrous toluene. Anhydrous toluene was used because the small amount of H₂O present in regular toluene may lead to polymerized products that would chemsorb to the surface. It is competitive with silanized products⁷. In the case of APTES, extensive crosslinking may occur in bulk solution prior to surface attachment, causing the surface layer to contain ill-defined silane aggregates and an uncontrolled structure.

After silane reaction with the silica surface, the surfaces are typically post-treated by a heat treatment for several hours to overnight at 100° C. The heat treatment helps convert physically adsorbed (e.g. via hydrogen bonding between the amine group and surface silanols) silane molecules to chemically bonded species. Otherwise, washing procedures will remove the physically adsorbed silane, causing a loss in coverage. In contrast, chemically bonded molecules are more stable and won't cleave from the surface as easily in subsequent surface modification, described next.

Heterobifunctional Crosslinkers. Three types of heterobifunctional crosslinkers were used in the second step of surface modification. The crosslinkers used are p-maleimidophenyl isocyanate (PMPI), m-maleimidobenzoyl-N-hydroxysuccinimide ester (MBS), and N-(γ-maleimidobutyryloxy)sulfosuccinimide ester (Sulfo-GMBS). All three crosslinkers are commercial products from Pierce Biotechnology.

Because heterobifunctional crosslinkers possess two selectively reactive groups that allow coupling to be carried out in a stepwise manner, better control of the conjugation chemistry is attainable. For example, in a situation in which two molecules A and B are to be covalently linked together, A may be reacted with the crosslinker first, purified, and characterized, before carrying out the second reaction with a molecule of B.

The crosslinker PMPI has the structure

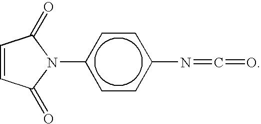

The isocyanate (—N═C═O) group is a highly reactive, sterically unencumbered functionality that will react with amines to form ureas and with alcohols to form carbamates. The other reactive site in PMPI is a maleimide (on the left in above structural image), which under conditions of near neutral pH exhibits about a 1000-fold selectivity for reaction with thiol (—SH) groups over amines. Thus, PMPI constitutes a net amine-to-thiol coupling.⁸ In Example 3, PMPI is used to join sulfhydryl-containing oligonucleotides to amine groups on silane-modified supports. Annunziato et al⁸ describe the synthesis of PMPI.

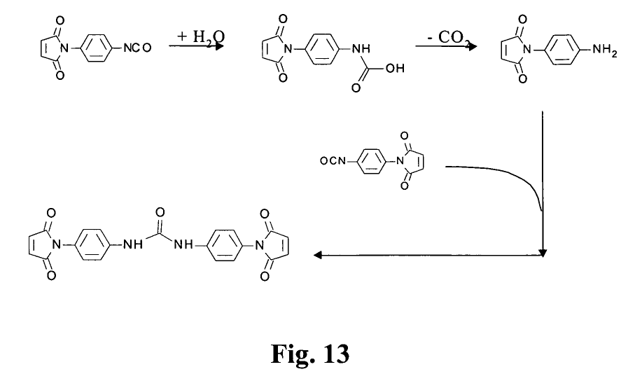

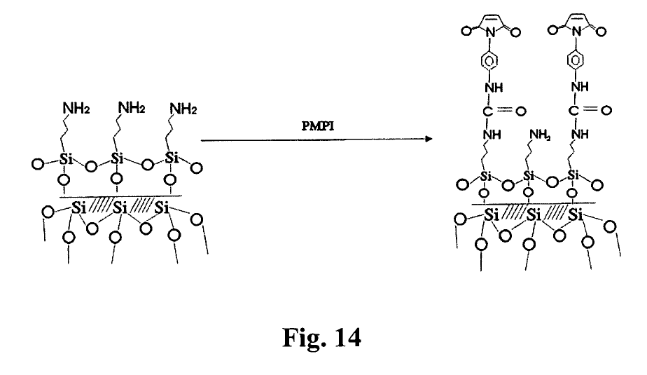

Isocyanates are extremely unstable in water and therefore PMPI reaction with surface amines was performed in dry organic solvents such as anhydrous acetonitrile. In those cases where water might be present in the reaction mixture (either surreptitiously or in the form of hydrated reactants) excess PMPI must be added. Water reacts with PMPI to yield N,N′-bis(p-maleimidophenyl)urea, a bismaleimide homobifunctional cross-linking reagent (FIG. 13). When modifying siliceous surfaces, some water is expected to be present in the form of surface-bound species, which may lead to deactivation of a portion of the added PMPI. Once all water is consumed, however, excess PMPI is free to react with surface amine or other groups. In the case of reaction with an aminosilylated surface, the surface will obtain the maleimide functionality (FIG. 14).

Both the isocyanate and maleimide groups on PMPI are reactive toward nucleophiles such as amines. This leaves the orientation of the PMPI molecule following surface attachment (FIG. 14) open to question. Example 3 shows that attachment through the isocyanate site on the PMPI is the dominant reaction. This immobilization orientation is the desired outcome.

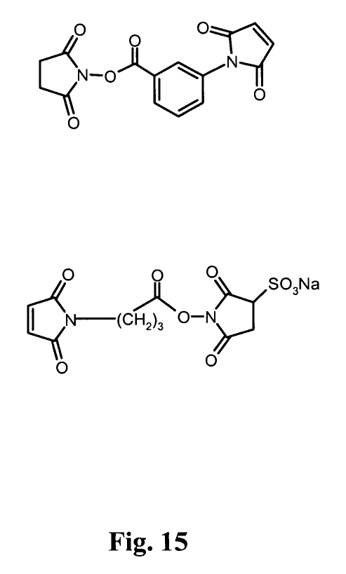

In addition to PMPI, the heterobifunctional crosslinkers MBS and sulfo-GMBS have also been tried. The structure of these molecules is shown in FIG. 15. As is the case with PMPI, both MBS and sulfo-GMBS have a maleimido group that may react with a thiol moeity. In contrast to PMPI, their amine-reactive site is not an isocyanate but instead is an N-hydroxysuccinimide ester (NHS-ester). During reaction, an amine group attacks the NHS ester carbonyl to yield an amide linkage. The presence of the sulfonate group on sulfo-GMBS makes this linker water soluble, while MBS is not water soluble.

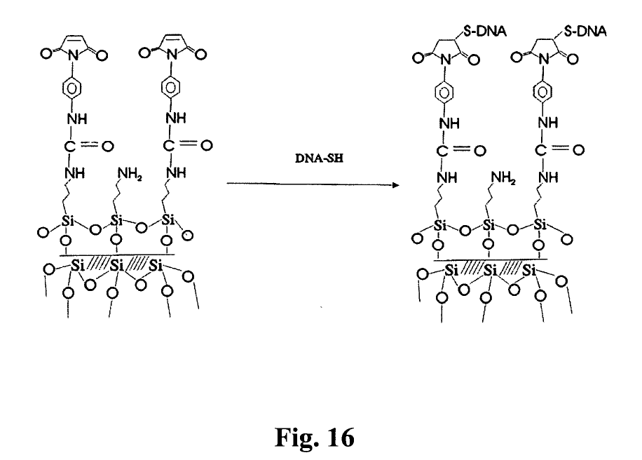

Oligonucleotides. For investigation of surface attachment of single-stranded DNA and its activity toward hybridization four different oligonucleotides were used. The first one, referred to as the thiol probe, was a disulfide-terminated 20mer oligonucleotide whose thiol group, after reduction of the disulfide, was used to tether it to surface maleimides introduced by the PMPI, MBS, or sulfo-GMBS crosslinkers (see preceding section). See FIG. 16. The second oligonucleotide (probe control) was identical to the thiol probe except for the disulfide modification. As it lacked the thiol group, the probe control was used to test for site specificity of attachment. The third 20mer oligonucleotide was a fluorescently labeled target, with a sequence fully complementary to that of the probes, used to test the hybridization activity of surface tethered probe strands. The fourth and last oligonucleotide was also fluorescently labeled but its 20mer sequence was noncomplementary to the probe sequence. This target was used to validate sequence specificity of hybridization. All oligonucleotides were purchased from Qiagen Inc.

The disulfide bond at the 3′-end of probe strands had to be cleaved to generate a free thiol for attachment to surface maleimides. The cleaving procedure is described in Appendix II.4.i. Commonly, two methods are used for reductive cleaving of disulfides: one employs dithiothreitol (DTT) as the reducing agent and the other uses tris(2-carboxyethyl)phosphine (TCEP). The disulfide containing oligonucleotide is treated with the reagent of interest (DTT or TCEP) under proscribed conditions, followed by separation of excess DTT or TCEP from cleaved oligonucleotides on a size-exclusion column (PD-10 from Amersham Biosciences).

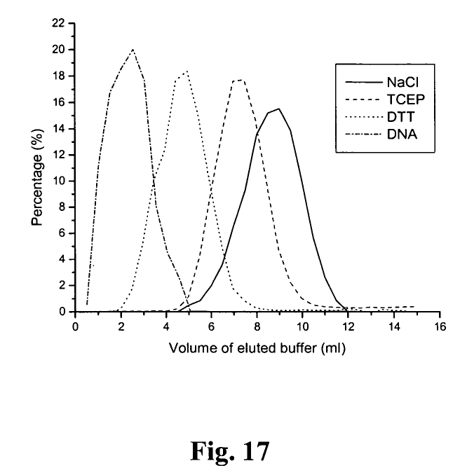

The quality of size separation of oligonucleotides from excess DTT or TCEP on PD-10 columns was evaluated. Elution profiles of the cleaved 20mer, DTT, TCEP, and NaCl were compared. FIG. 17 shows that all of the DNA went through the column in the first four ml. Most of the DTT eluted between the 5th and 10th ml. Compared to DTT, TCEP had a greater overlap with DNA elution profile as it passed through the column between the 3rd and 7th ml. NaCl elutes last, between 7th to 11th ml.

From FIG. 17, separation of DTT from a DNA 20mer is cleaner than separation of TCEP. Therefore, in this dissertation, DTT was used in preference to TCEP. To further ensure a clean separation of DNA 20mers from DTT only the first 3 ml coming off the PD-10 column were collected. The reason for separating the oligonucleotide from the reducing reagent (such as DTT or TCEP) is that both DTT and TCEP compete with the DNA thiol for reaction with surface maleimide groups^{9,10}.

The moieties on a nucleic acid strand make it capable of forming hydrogen bonds as well as undergo electrostatic or hydrophobic interactions. The complexity of possible interactions makes control of conformation of surface attached DNA strands problematic (e.g. nucleic bases may hydrogen bond with surface amines). When placed under an aqueous environment, in one embodiment, it is desirable for the strands to dangle out into solution rather than lie down on the surface. Certain steps may be taken to control these interactions. For example, to eliminate the problem of electrostatic interactions 1 M salt solutions may be used^{11,12}. In addition to influencing interactions between a strand and the surface, salt conditions will also affect electrostatic interactions between neighboring surface bound strands. A number of theoretical studies of such polyelectrolyte monolayers have been reported¹³⁻¹⁵.

In the following section, characterization techniques used to study the modified surfaces are described.

Characterization Methods to Study Modified Surfaces

Transmission FTIR. A Fourier Transform Infrared (FTIR) spectroscopy instrument, Nicolet Magna-IR 560, was used. This instrument has an MCT-A detector, an Everglo™ Infrared light source, and uses a standard KBr beam splitter in the interferometer. The instrument detects in the mid-IR range from 4000-650 cm⁻¹. Typical settings used in experiments were to average 1000 scans, taken at a resolution of 4 cm⁻¹, with a 150 aperture setting. The larger the aperture setting, the higher the signal to noise ratio of the collected spectra. Since the signal intensity from the studied samples was relatively low, the maximum aperture setting of 150 was employed.

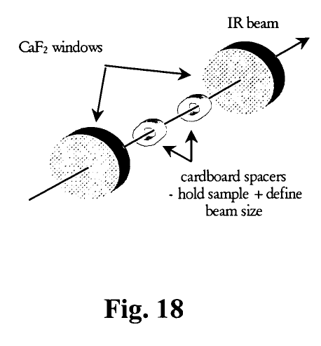

FTIR was used to quantitatively determine how many silane and linker molecules are attached to fumed silica supports per unit area. This was accomplished by using the absorption of the material of interest at a unique frequency in the IR to determine how much of that material is present. These measurements only worked with high surface area fumed silica (Aerosil® 200) supports since the silane and linker layers are only a few nm thick, affording too little material to detect accurately on planar supports. Powder silica spectra were measured in transmission (FIG. 18). By taking advantage of the high specific surface area of Aerosil® 200, direct transmission through powder specimens was used to measure absorption due to immobilized organic (silane or linker). DNA attachment was quantified fluorescently (see below), while FTIR was used for investigation of the first two steps of attachment, i.e. silanization and crosslinker immobilization.

In the FTIR measurements, four scans of Intensity B₁, B₂, S₁ and S₂ were used for each sample. The purpose of these scans is:

B₁—Background scan at 0 minutes. The background was measured with just air in the sample holder (see further description under S₁). The FTIR sample chamber has to be opened to put the holder in. The time that chamber is sealed again is marked as 0 minutes.

B₂—Background scan at 10 minutes. This was a second scan repeated on the sample holder just filled with air. During the 10 minutes between the B₁ and B₂ scans, the sample compartment is continually purged resulting in a decrease of water vapor and CO₂ absorptions from the atmosphere in the compartment. The compartment is kept sealed during this time. By ratioing B₂ and B₁, spectra of water vapor is obtained which is subtracted from the sample spectra (S₂) to allow observation of features that would otherwise be hidden under the water absorptions.

S₁—Sample scan at 0 minutes. After the B₁ and B₂ scans, the sample holder is filled with powder and reinserted into the sample compartment. To fill the holder, approximately 2-3 mg of Aerosil® 200 powder are sandwiched between two CaF₂ windows using a cardboard cutout mask with a 3 mm diameter opening (FIG. 18). The mask and windows are held together inside a commercial cell designed for obtaining spectra of liquid samples. Depending on measurement either bare, silylated, or linker modified powder was characterized. The S₁ scan is simply used to confirm that the measurement is proceeding normally.

S₂—Sample scan at 10 minutes. After 10 minutes of purging the sample compartment, which is kept sealed during this time, the S₂ scan is taken.

Each of the above scans (B₁, B₂, S₁, and S₂) consists of the average of a 1000 individual scans, at the same resolution of 4 cm⁻¹. The transmission spectrum T of the powder is calculated as follows:

$$T = \frac{{S}_{2}}{{B}_{2}} - k * \frac{{B}_{2}}{{B}_{1}} \qquad ( eq . 2.1 )$$

where the value of k is optimized to eliminate the H₂O vapor contributions. The absorption A is calculated from the transmission using
A=−log(T). (eq. 2.2)

UV Absorption. Another method used extensively is ultraviolet-visible (UV-Vis) spectroscopy. The spectrophotometer used is a Varian Cary™ 50. It has a single Xenon light source and Varian Cary™ 50 photomultiplier. It has wavelength range from 190 to 1100 nm, and sensitivity of 0.001 Absorbance units.

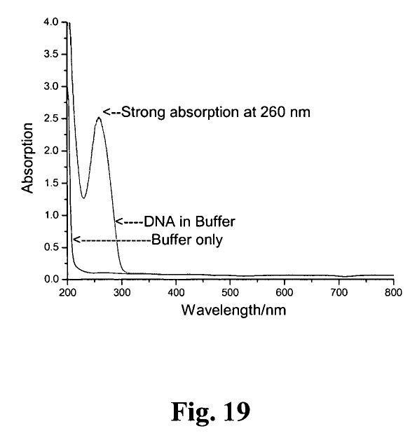

DNA absorbs strongly at 260 nm as shown in FIG. 19. This absorption is used to determine the concentration of DNA solutions. In particular, the following equation is employed to measure oligonucleotide concentration,
A=εML (eq. 2.3)
where A is the measured absorbance, ε is the molar absorption coefficient of the oligonucleotide (M⁻¹ cm⁻¹), M is molarity (unknown) of the DNA solution (mol/L), and L (cm) is the path length traversed by the UV beam through the DNA solution. The molar absorption ε varies with base sequence, as each type of nucleotide absorbs differently. Thus, for each deoxyadenine (dA) nucleotide present in a sequence the contribution to ε is 15400 M⁻¹ cm⁻¹, for dC it is 7400 M⁻¹ cm⁻¹, for dT it is 8700 M⁻¹ cm⁻¹, and for dG it is 11500 M⁻¹ cm⁻¹. These values apply to 260 nm, 25° C., in 0.1 molar ionic strength, and pH 7¹⁶. The thiol probe sequence will be used to illustrate. Its sequence is 5′-CGT TGT AAA ACG ACG GCC AG-S-S-R-3′ (SEQ ID NO. 1), or 5 C's, 6 G's, 3 T's, and 6 A's. Given the above extinction coefficients, the molar absorption coefficient ε of the entire sequence is ε=5×7400+6×11500+3×8700+6×15400=224500 M⁻¹ cm⁻¹. There are also more complicated, but more accurate, methods of estimating ε¹⁶. Molar absorption coefficients of the oligonucleotides are supplied by Qiagen.

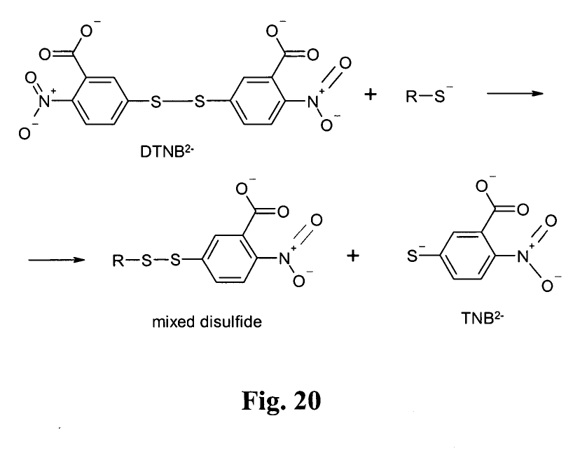

Ellman's Assay. Ellman's reagent¹⁷ 5,5′-dithio-bis-(2-nitrobenzoic acid) (DTNB) is used for measuring the concentration of free sulfhydryl (i.e. thiol) groups in solution. A solution of this water-soluble compound will turn yellow when contacted with free thiols, a characteristic exploited in the DTNB assay. DTNB is very specific towards —SH groups at neutral pH values, has a high molar extinction coefficient, and reacts very fast. The molecular structure of DTNB is shown in FIG. 20.

DTNB reacts with a free sulfhydryl group to yield a mixed disulfide and 2-nitro-5-thiobenzoic acid (TNB) (FIG. 20). The target of DTNB in this reaction is the conjugate base (R—S—) of a free sulfhydryl group. TNB is the “colored” species produced in this reaction, and has a high molar extinction coefficient of 14,150 cm⁻¹ M⁻¹ at 412 nm^{18,19}. The extinction of TNB is not affected by changes in pH between 7.6 and 8.6.

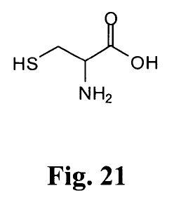

Ellman's assay was used to determine the surface coverage of maleimide groups on linker-modified Aerosil® 200. The general procedure consists of first reacting cysteine, which contains a thiol group in its side chain (see FIG. 21), with surface maleimides on functionalized Aerosil®. The amount of powder is carefully premeasured, and hence the total amount of surface being titrated with cysteine is known. The decrease in cysteine concentration in bulk solution, resulting from consumption of cysteine by reaction with surface maleimides, is determined via Ellman's assay. If each maleimide is assumed to consume one cysteine, the measured decrease in bulk cysteine concentration may be directly translated to coverage of maleimides (i.e. active maleimide groups per nm²) on the powder. Operational details of this procedure are provided in Appendix II.4.ii. of this Example.

Fluorometry. Fluorometry was used to quantify the concentration of a fluorescently-tagged species. The fluorometer SLM 8000C Spectrofluorometer from SLM Instruments, Inc. was used. This instrument has a 450-watt ozone-free xenon arc lamp as the source, emission and excitation monochromators, and a photon-counting detector. Both the emission and excitation monochromators have a wavelength accuracy of ±0.2 nm. The accessible wavelength range for excitation and emission is from 300 nm to 900 nm.

The principal use of fluorometry in this work was to detect changes in concentration of fluorescently labeled DNA solutions. For example, as described in the next section, OliGreen® staining kits from Molecular Probes were used to monitor changes in DNA probe concentration resulting from surface attachment of the probe. For OliGreen® measurements, the settings used were 480 nm excitation wavelength and 520 nm emission (detection) wavelength. When needed, measurement of changes in concentration of fluorescein-labeled DNA targets was accomplished with 494 nm excitation and 520 nm emission. Appendix II.4.iii provides operational details of the fluorometer instrument.

Quantification of Single-Stranded DNA Solutions with OliGreen®. OliGreen® ssDNA Quantitation Kit was used to determine concentrations of single-stranded DNA. For probe molecules, comparison of the DNA concentration in bulk solution before and after attachment allowed determination (by difference) of the amount of ssDNA attached. The OliGreen® reagent binds quickly and selectively to single stranded DNA to form a fluorescent complex²⁰. Under the conditions of usage, the amount of fluorescence intensity observed is proportional to the concentration of single-stranded DNA present in the solution.

Notably, as described earlier, the most common technique for measuring DNA concentrations is by determination of absorbance at 260 nm (A₂₆₀). However, absorbance measurements are not as sensitive as fluorometry. At a practical absorbance limit of about 0.01, UV-Vis detects DNA concentration of about 0.3 μg/ml. In contrast, OliGreen® enables measurement of as little as 3 ng/ml. This sensitivity exceeds that achieved with absorbance methods by about 100 fold.

Elemental Analysis. Elemental analysis was used to determine amounts of organic attached to fumed silica supports. These results were used to calibrate FTIR absorbance measurements, so that the surface coverages (molecules/area) of silane and linker could be determined from infrared spectra. Powder Aerosil® 200 samples were sent to Galbraith Laboratories for elemental analysis.

In elemental analysis, the sample to be analyzed is weighed very accurately to 1 μg inside a small tin capsule. The capsule is combusted to form tin oxide, elevating the temperature so that the sample undergoes complete combustion to form CO₂, N₂, NₓO_{y}, H₂O and other by-products. The ‘quantity’ of each gas, CO₂, H₂O, N₂ and He carrier gas are recorded. From these readings, and the weight of sample used, the sample composition in terms of % C, % H, and % N are calculated and expressed as a fraction of the total initial mass of the specimen.

The following table presents an example of elemental analysis results obtained for Aerosil® powder with just APTES attached (sample MBS7), with APTES and a small amount of MBS linker (sample MBS1), and with APTES and a large amount of MBS (sample MBS6).

TABLE 2.1
Example of Elemental Analysis Results for MBS on APTES
modified Aerosil ®.

| | | | | | Coverage |
|---|---|---|---|---|---|
| | % C | % N | % H | % SiO₂ | (molecules/nm²) |
| MBS1 | 1.7 | 0.43 | 0.60 | 97.27 | 0.040 MBS |
| MBS6 | 3.72 | 0.93 | 0.62 | 94.73 | 0.54 MBS |
| MBS7 | 1.53 | 0.60 | 0.57 | 97.30 | 1.3 APTES |

In Table 2.1, the % C and % H are given by weight. % N by weight was calculated from % C by knowing the ratio of nitrogen and carbon atoms in immobilized molecules. % SiO₂ is equal to 100% minus the sum of % C, % N and % H by weight. We assume the ratio of MBS to APTES is 1:1 as we calculating the % N, which is the maximum ratio of MBS to APTES. Since % N is less than 1% of % SiO₂ and it is only used in the calculation of % SiO₂, percent error is less than 1%.

For example, to calculate APTES coverage from the MBS7 (APTES modified powder) data in Table II.1 the following equation would be used:

$$Coverage ( molecules \text{/} {nm}^{2} ) = \frac{\frac{\% C}{12 * 3} * {N}_{A}}{\% {SiO}_{2} * 200 * {10}^{18}} \qquad ( eq 2.4 )$$

where 12 is the molecular weight of carbon, 3 is number of carbon atoms in each attached APTES (see FIG. 11), 200 is the surface area of Aerosil® 200 in m²/g, and 10¹⁸ is the conversion from m² to nm². Using the MBS7 data, the APTES coverage is calculated to be 1.3 molecules/nm².

To calculate coverage of the MBS linker, the % C measured (e.g. in samples MBS1 or MBS6) must first be corrected by subtracting that due to APTES. Thus, for MBS6, the % C attributed to the linker is % C=3.72−1.53=2.19. This corrected % C value is then used in equation 2.5 to calculate the coverage of MBS linker,

$$Coverage ( molecules \text{/} {nm}^{2} ) = \frac{\frac{\% C}{12 * 11} * {N}_{A}}{\% {SiO}_{2} * 200 * {10}^{18}} \qquad ( eq 2.5 )$$

In equation 2.5, 12 represents the molecular weight of carbon, 11 is the number of carbon atoms in an attached MBS residue, 200 is surface area of Aerosil® in m²/g, and 10¹⁸ converts from m² to nm². The result, for MBS6, comes to 0.54 MBS molecules/nm² (see Table II.1).

Appendices.

II.4.i. Cleavage of Disulfides to Generate Free Thiol Groups. The following procedure uses DTT as the disulfide reducing agent.

1. Make 100 ml 10 mM KH₂PO₄ and 1 M NaCl buffer and adjust pH to 7.0 by adding appropriate amount of 1 N HCl to decrease pH or 1 N NaOH to increase pH while monitoring using a pH meter.

2. Make 5 ml 10 mM KH₂PO₄ and 1M NaCl buffer and adjust pH to 8.0.

3. Mix 0.05-0.2 ml of 0.2 mM disulfide-DNA, 1 mg DTT, and 1 ml pH 8.0 phosphate buffer in 1.5 ml microcentrifuge vial, and let react for 1 hour. 1 mg DTT is 163 molar excess over 0.2 ml of 0.2 mM disulfide-DNA solution.

4. While DTT is cleaving the DNA, run 25 ml of pH 7.0 phosphate buffer through PD-10 column, then cap the column.

5. Dilute the 1 ml of cleaved DNA solution to 2.5 ml with pH 7.0 phosphate buffer.

6. Load the 2.5 ml DNA solution on the PD-10 column and discard the first 2.5 ml of effluent.

7. Load 3.0 ml pH 7.0 phosphate buffer on the PD-10 column, and collect all 3.0 ml of effluent. The effluent contains the cleaved DNA, now separated from the remnant DTT.

II.4.ii. Determination of Surface Coverage of Maleimide Groups on Fumed Silica Supports/Ellman's Assay²¹. The following procedure describes titration of maleimide derivatized fumed silica with L-cysteine. Based on spectrophotometrically determined decrease (performed via Ellman's assay) in the concentration of L-cysteine in bulk solution, the surface coverage of active maleimides is determined.

1. Make 200 ml of 1 mM EDTA, 100 mM NaCl, 10 mM KH₂PO₄ buffer and adjust pH to 7.2 by adding appropriate amount of 1 N HCl to decrease pH or 1 N NaOH to increase pH while monitoring with a pH meter. Degas the buffer by putting the buffer bottle in the sonicator with cap open for 10 minutes in degas mode. This buffer will be referred to as the pH 7.2 buffer.

2. Make 50 ml of 5 mM sodium acetate, 50 mM NaCl, 0.5 mM EDTA and adjust pH to 4.7 by adding appropriate amount of 1 N HCl to decrease pH or 1 N NaOH to increase pH while monitoring using a pH meter. Degas by putting buffer bottle in the sonicator with cap open for 10 minutes in degas mode. This buffer will be referred to as the pH 4.7 buffer.

3. Accurately weigh out 1.0 mg of maleimide-modified powder into a 1.5 ml Eppendorf centrifuge vial. Note: for necessary confidence in the determination it is highly recommended that each measurement is performed in duplicate (i.e. 2 vials with 1 mg of powder in each for each powder sample to be characterized).

4. To each of the vials containing sample add 950 μL of pH 7.2 buffer. Note: to prevent oxidation of the L-cysteine thiol all solutions used in the Ellman's assay should be de-gassed using the sonicator bath (degas setting; as described above for the buffer solutions). Also prepare two “blank” vials without powder but with 950 μL of pH 7.2 buffer added.

5. To each vial, including blanks, add 20 μL of a freshly prepared, de-gassed 77 mM L-cysteine stock solution prepared in the pH 4.7 de-gassed buffer. 20 μL of 77 mM L-cysteine stock added to 1 mg of Aerosil® 200 powder is equivalent to 5 molecules of L-cysteine added per nm² of powder surface.

6. As an experimental control, add 950 μL of pH 7.2 buffer and 20 μL of pH 4.7 (note: no cysteine) buffer to an empty Eppendorf vial. Make-up in triplicate (e.g. using a total of three vials).

7. Allow 1 hour for L-cysteine to react with surface maleimides (or just mix with buffer in the case of the blanks) at room temperature with agitation (vortexer setting 7). Note: shorter reaction times e.g. 15-30 mins are typical but some of the maleimides on the powder may be relatively inaccessible. Initial results indicate that if sufficient amount of L-cysteine is added to each centrifuge tube containing maleimide-modified powder, then the color of the maleimide-modified powder will change from yellow to white as the L-cysteine thiols react with the maleimides and the conjugation is lost.

8. Add 20 μL of a 385 mM de-gassed stock solution of Ellman's reagent (DTNB) in DMSO to every vial (blanks and controls as well). Note: It is important that the volume of DMSO added to the tubes is kept as small as possible (e.g. ca. 20 μL).

9. Allow all vials to mix on the vortexer for 10 minutes (setting 7). During this time, DTNB will react with any L-cysteine present. Then centrifuge the powder vials for 10 mins at 10,000 rpm. During centrifuging of powder vials, keep blanks and controls aside (not on vortexer).

10. Measure the absorbance of the supernatants (being careful not to disturb the powder on the bottom of the centrifuge tubes) at 412 nm using plastic, disposable, UV-transparent restricted-volume cuvettes. Note: the solutions to be measured include the powder samples as well as blanks and controls. Absorbances should be in the 0-1.0 Abs units range—dilute samples as necessary with pH 7.2 buffer to lower absorbance (e.g. 20 μL sample+0.95 ml pH 7.2 buffer), keeping careful note of the amount of buffer added. Typically, same dilution factor would be applied to all samples, blanks, and controls.

11. Average the duplicate absorbance values for each sample and blanks (and the triplicate values for the controls) correcting for any dilution used to adjust absorbance in step 10. For example, multiply the measured absorbance by 48.5 in the case of a 20 μL sample that has been diluted with 0.95 ml pH 7.2 buffer.

12. The following equation is used to calculate the amount of maleimide on a powder sample:

$$molecules \text{/} {nm}^{2} = \frac{\begin{matrix}( {UV}_{Control} - {UV}_{Sample} ) * \\ {N}_{A} * 0.00099 l\end{matrix}}{\begin{matrix}14150 {M}^{- 1} {cm}^{- 1} * 0.001 g * \\ 2 * {10}^{20} {nm}^{2} \text{/} g * 1 cm\end{matrix}} \qquad ( eq . 2.6 )$$

where UV_{Control} is the UV absorption of dilution-corrected control at 412 nm; UV_{Sample} is the UV absorption of dilution-corrected sample at 412 nm; 0.00099 l is total volume of 0.95 ml pH 4.7 buffer, 20 μL-cysteine stock solution, and 20 μL Ellman stock solution; 14150 M⁻¹ cm⁻¹ is the extinction coefficient of DTNB at 412 nm; 0.001 g is the amount of powder used; 2×10²⁰ nm²/g is the surface area of Aerosil® 200 per mass; 1 cm is the path length of the UV cell.
For example: if UV_{Control}=0.338*48.5=16.393, UV_{Sample}=0.253*48.5=12.271, then eq. 2.6 yields:

$$\begin{matrix}molecules \text{/} {nm}^{2} = \frac{\begin{matrix}( {UV}_{Control} - {UV}_{Sample} ) * \\ {N}_{A} * 0.00099 l\end{matrix}}{\begin{matrix}14150 {M}^{- 1} {cm}^{- 1} * 0.001 g * \\ 2 * {10}^{20} {nm}^{2} \text{/} g * 1 cm\end{matrix}} \\ = \frac{\begin{matrix}( 16.393 - 12.271 ) * 6.022 \times \\ {10}^{23} molecules \text{/} mol * 0.00099 l\end{matrix} l}{\begin{matrix}14150 {M}^{- 1} {cm}^{- 1} * 0.001 g * \\ 2 * {10}^{20} {nm}^{2} \text{/} g * 1 cm\end{matrix}} \\ = 0.868 molecules \text{/} {nm}^{2}\end{matrix} \qquad ( eq . 2.7 )$$

Therefore, the coverage of active (reactive) maleimides is 0.868 molecules/nm².

### References for Example 2

1. Freeman, W. M., Robertson, D. J. & Vrana, K. E. Fundamentals of DNA hybridization arrays for gene expression analysis. BioTechniques 29, 1042-1055 (2000).
2. Schena, M. et al. Microarrays: Biotechnology's discovery platform for functional genomics. Trends in Biotechnology 16, 301-306 (1998).
3. Lockhart, D. J. & Winzeler, E. A. Genomics, gene expression and DNA arrays. Nature 405, 827-836 (2000).
4. Barthel, H., L. Roesch, and J. Weis, Fumed silica—production, properties, and applications. Organosilicon Chem. II, [Muench. Silicontage], 2nd, 1996: p. 761-778.
5. Morterra, C., G. Magnacca, and V. Bolis, On the critical use of molar absorption coefficients for adsorbed species: the methanol/silica system. Catalysis Today, 2001. 70(1-3): p. 43-58.
6. Moon, J. H., et al., Absolute surface density of the amine group of the aminosilylated thin layers: Ultraviolet-visible spectroscopy, second harmonic generation, and synchrotron-radiation photoelectron spectroscopy study. Langmuir, 1997. 13(16): p. 4305-4310.
7. White, L. D. and C. P. Tripp, Reaction of (3-aminopropyl)dimethylethoxysilane with amine catalysts on silica surfaces. Journal of Colloid and Interface Science, 2000. 232(2): p. 400-407.
8. Annunziato, M. E., et al., P-Maleimidophenyl Isocyanate—a Novel Heterobifunctional Linker For Hydroxyl to Thiol Coupling. Bioconjugate Chemistry, 1993. 4(3): p. 212-218.
9. O'donnell, M. J., et al., High density, covalent attachment of DNA to silicon wafers for analysis by MALDI-TOF mass spectrometry. Analytical Chemistry, 1997. 69(13): p. 2438-2443.
10. Getz, E. B., et al., A comparison between the sulfhydryl reductants tris(2-carboxyethyl)phosphine and dithiothreitol for use in protein biochemistry. Analytical Biochemistry, 1999. 273(1): p. 73-80.
11. Chrisey, L. A. et al. in Materials Research Society Symposium Proceedings; eds.
12. Alper, M., Bayley, H., Kaplan, D. & Navia, M. 179-184; Materials Research Society, Pittsburgh, Pa., 1994
13. Israels, R., et al., Charged Polymeric Brushes—Structure and Scaling Relations. Macromolecules, 1994. 27(12): p. 3249-3261.
14. Zhulina, E. B., R. Israels, and G. J. Fleer, Theory of Planar Polyelectrolyte Brush Immersed in Solution of Asymmetric Salt. Colloids and Surfaces a-Physicochemical and Engineering Aspects, 1994. 86: p. 11-24.
15. Zhulina, E. B., O. V. Borisov, and T. M. Birshtein, Polyelectrolyte brush interaction with multivalent ions. Macromolecules, 1999. 32(24): p. 8189-8196.
16. Cantor, C. R., Oligonucleotide Interactions. III. Circular Dichroism studies of the Conformation of Deoxyoligonucleotides. Biopolymers, 1970. 9: p. 1059-1077.
17. Ellman, G. L. Tissue sulfhydryl groups. Archives of Biochemistry and Biophysics, 1959. 82, p. 70-77
18. Eyer, P., et al., Molar absorption coefficients for the reduced Ellman reagent: reassessment. Analytical Biochemistry, 2003. 312(2): p. 224-227.
19. Riddles, P. W., et al., Ellman's reagent. 5,5′-dithiobis(2-nitrobenzoic acid)-a reexamination. Analytical Biochemistry, 1979. 94: p. 75-81
20. Reyderman, L. and S. Stavchansky, Determination of single-stranded oligodeoxynucleotides by capillary gel electrophoresis with laser induced fluorescence and on column derivatization. Journal of Chromatography a, 1996. 755(2): p. 271-280.
21. Singh, R., A Sensitive Assay For Maleimide Groups. Bioconjugate Chemistry, 1994. 5(4): p. 348-351.

### EXAMPLE 3

### Immobilization of Nucleic Acids on Siliceous Surfaces Using Heterobifunctional Crosslinkers

III.1. Introduction. Nucleic acids immobilized on solid supports are widely employed in biological and medical diagnostics¹⁻³, as well as stand to provide fundamental insight into chemically and physically adsorbed polymer layers⁴⁻¹². The properties of such films are closely coupled to the geometry and topology with which the molecules are attached to the interface. DNA has been immobilized on a variety of materials including metals, glass and polymers¹³⁻¹⁵. When covalent attachment of DNA to glass or silica (jointly referred to as siliceous) surfaces is desired, the usual procedure involves silylation of the surface to introduce amine, epoxy, thiol, or other functional groups, followed by either direct reaction with the nucleic acid of interest or with an intermediary crosslinker molecule to which DNA is subsequently attached. Often, a specific moiety is introduced on the DNA to improve site-specificity of immobilization, such as a terminal primary amine or thiol group. Nevertheless, side reactions with exocyclic base amines or other groups on the DNA are possible, and may interfere with regiospecificity of attachment¹⁶.

A common method for immobilizing oligonucleotides to siliceous supports involves surface modification with maleimide groups¹⁷⁻²². Oligonucleotides modified with terminal thiols then covalently attach through thioether linkages formed by reaction with the maleimide C═C double bond. Success of the multiple synthetic steps is often difficult to confirm due to challenges in characterizing surfaces bearing monolayer or submonolayer films. Therefore, in this Example, fumed silica powder supports were employed in order to exploit their high surface to volume ratio to better validate the surface chemistry. As previously set forth, there are typically three steps in the modification of a siliceous surface with a biological macromolecule: (i) self-assembly of an organosilane layer to introduce reactive chemical moieties to the surface, (ii) if needed, reaction of the silane layer with appropriate crosslinkers to introduce functional groups with greater or more specific reactivity toward biomolecules, and (iii) immobilization of the biomolecule of interest.

In this Example, use of two aminosilanes, (3-aminopropyl)dimethylethoxysilane (APDMES) and 3-aminopropyltriethoxysilane (APTES), is explored in combination with crosslinkers employing either isocyanate or N-hydroxysuccinimide ester (NHS-ester) moieties as amine-reactive sites and maleimides as thiol-reactive sites. These crosslinkers are designed to attach thiolated biomolecules to aminated surfaces. APDMES was found to possess poor stability, making APTES the aminosilane of choice. Moreover, an isocyanate bearing crosslinker was found much more effective than NHS-ester analogs in blocking undesirable side reactions, despite the wide use of NHS-ester linkers in the literature. However, neither isocyanate nor NHS-ester crosslinkers produced monolayers with a 1:1 stoichiometry to surface amines. Immobilization and hybridization of oligonucleotides was also studied, and suggested additional directions for improvement. One characteristic of this work is that each modification step has been examined within a single study, affording insights into successful preparation of robust and functional biomolecular monolayers. The results of this work also impact upon protein and peptide modified surfaces, as these are often formed by like methods²³⁻²⁵.

III.2. Experimental Background.

III.2.i. Materials. Details of materials used were provided in Example 2. Briefly, Aerosil® 200 fumed silica was a commercial sample from Degussa-Hüls. This powdery material has a BET surface area of 200±25 m²/g and consists of aggregates of 12 nm diameter primary silica particles. Independent confirmation of the BET specific area yielded 199.97 m²/g (Micromeritics Instrument Corp.). Content of the fumed silica is at least 99.8% amorphous SiO₂. Aminopropyltriethoxysilane (APTES; 98%) was purchased from Aldrich. (3-aminopropyl)dimethylethoxysilane (APDMES; 95%) was purchased from Gelest, Inc. Heterobifunctional crosslinkers p-maleimidophenyl isocyanate (PMPI), m-maleimidobenzoyl-N-hydroxysuccinimide (MBS), and N-(γ-maleimidobutyryloxy)-sulfosuccinimide ester (sulfo-GMBS) were obtained from Pierce Biotechnology, as was Ellman's reagent (DTNB; 5,5′-dithio-bis-(2-nitrobenzoic acid)). Dithiothreitol (DTT; 99%), L-cysteine (98%), and N-phenylmaleimide (NPM; 97%) were from Aldrich. OliGreen® kit for quantification of single-stranded DNA concentrations was from Molecular Probes Inc. All materials were stored according to provider's instructions and used as received unless indicated otherwise. DNA oligonucleotides, purified by HPLC, were purchased from Qiagen Inc. The 20mer sequences used are listed in Table 3.1. The sequences were chosen not to have self-complementarity so as to avoid formation of secondary structure (e.g. hairpins).

TABLE 3.1
Oligonucleotide Sequences and Modifications

| | | |
|---|---|---|
| Abbr. | Sequence | Notes |

| | | |
|---|---|---|
| PSH | 5′-CGTTGTAAAACGACGGCCAG-(CH₂)₃- | 3′ thiol-modified probe |
| | SH-3′ | (sensing strand) |
| | (SEQ ID NO. 1) | |
| P | 5′-CGTTGTAAAACGACGGCCAG-3′ | Unmodified probe |
| | (SEQ ID NO. 1) | |
| TC | 5′-CTGGCCGTCGTTTTACAACG-FL-3′ | Complementary target to |
| | (SEQ ID NO. 2) | PSH, with 3′ fluorescein |
| | | label |
| TNC | 5′-CTAACTGTTACCTCGGTCGG-FL-3′ | Noncomplementary target, |
| | (SEQ ID NO. 3) | with 3′ fluorescein label |

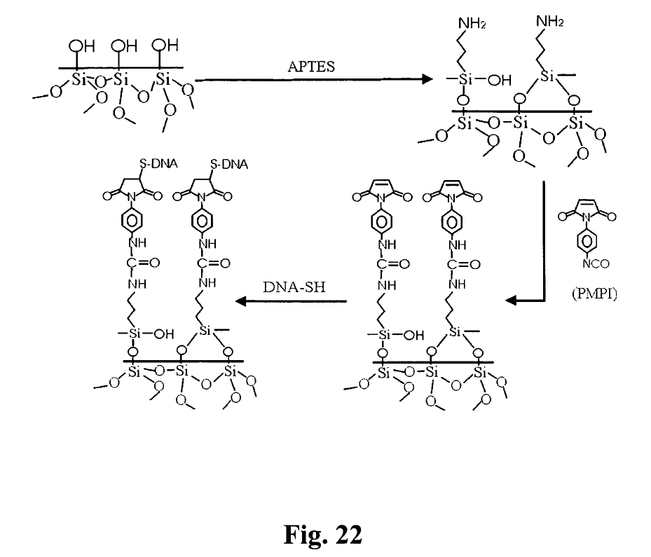

III.2.ii. Modification of Fumed Silica. The overall immobilization scheme is illustrated in FIG. 22. Typically, 400 mg of fumed silica was weighed into a polypropylene tube. A total of 13 g of 1% w/w solution APTES in anhydrous toluene was added and the mixture shaken for 30 minutes on a vortexer at room temperature. At the end of this period, the powder was centrifuged and the supernatant decanted. The powder was then washed by mechanically dispersing it in fresh solvent, centrifuging, and decanting. A sequence of two toluene washes, one deionized water (Millipore Biocell) wash, and one acetonitrile wash was used. For each wash, 10 ml of fresh solvent was added. Afterward the powder was dried overnight at 100° C. Typical recovery was 70%.

In a second step, the powder was modified with crosslinker, either PMPI or MBS. 20 mg APTES-modified silica was weighed into polypropylene 1.5 ml centrifuge vials and the desired concentration (between 0 and 0.01 w/w) of crosslinker in 1.4 ml anhydrous acetonitrile was added. Reaction was carried out for 30 minutes at room temperature while continually shaken on a vortexer. Acetonitrile was used as solvent due to prior reports of better suitability than N,N-dimethyl formamide, dimethyl sulfoxide, or aqueous buffers in similar applications²³. The powder was centrifuged and decanted, followed by two 1 ml washes with acetonitrile, one with saline phosphate buffer (PBS: 10 mM sodium phosphate, 1 M NaCl, pH 7), and a third acetonitrile wash. The powder was dried overnight at 50° C. unless noted otherwise. Both linkers are intended to produce a maleimide-enriched surface (FIG. 22). Demonstration of oligonucleotide attachment and hybridization was then performed using flat microscope slides, as described later in this Example.

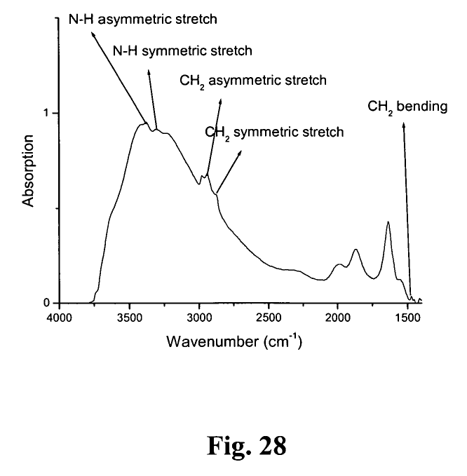

III.2.iii. Characterization of Modified Fumed Silica. Neat, APTES modified, and crosslinker-derivatized silica was examined by transmission infrared spectroscopy on a Nicolet Magna 560 IR spectrometer equipped with liquid nitrogen cooled MCT detector and by elemental analysis (Galbraith Laboratories). As detailed in the following sections, changes in total carbon content from elemental analysis were converted to coverage of APTES and crosslinker molecules and served to calibrate infrared absorption measurements. In powder spectra (e.g. see FIG. 28), scattering losses were often evident as a downward sloping baseline toward lower wavenumbers. At lower wavenumbers the incident wavelength is longer what enhances the scattering of the incident beam. In the following sections, the absorbance band assignments and calibrations that allowed estimates of surface coverage of silane and crosslinkers from IR spectra are described.

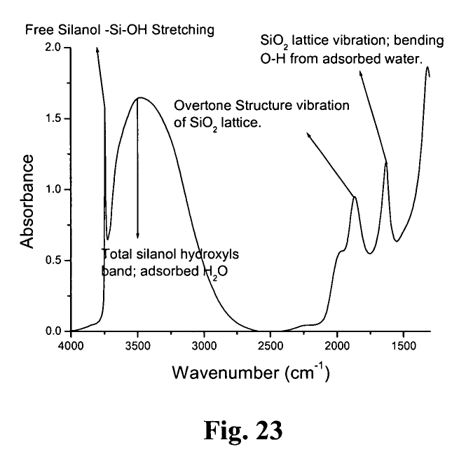

III.2.iii.a. Infrared Spectroscopy of Aerosil® 200. The commonly accepted infrared spectral assignments for silica are listed in Table 3.2^{26,27}. A transmission spectrum of Aerosil® 200 obtained on a 20 mg Aerosil® 200 pressed disk is displayed in FIG. 23. Pressed disks were prepared under 6 kbar of pressure applied for 10 minutes using Carver Hydraulic Press. Transmission through air was used as the background. The absorption peak at 3744 cm⁻¹ is due to isolated (i.e. not hydrogen bonded) surface silanol (—Si—OH) groups. The broad absorption between about 3700 cm⁻¹ and 3200 cm⁻¹ is due to various contributions, mainly entailing hydrogen bonded species. Thus intraglobular (buried inside the fumed silica particles) silanols are reported to absorb at 3650 cm⁻¹, while the maximum absorption for hydrogen bonded surface silanols occurs at 3520 cm⁻¹. In addition, molecularly physisorbed surface water absorbs in the general region 3400-3500 cm⁻¹. The peak between 1950-1766 cm⁻¹ is attributed to a silica skeletal overtone vibration, as is the peak around 1625 cm⁻¹, which however also possesses contributions from bending of surface physisorbed water (see below). At lower wavenumbers the onset of pronounced stretching absorptions of the —Si—O—Si— lattice is found, as are silanol bending modes. Quantitative spectral analysis was performed inside the region between 1300 cm⁻¹ and 3300 cm⁻¹.

TABLE 3.2
Principal IR Assignments for Aerosil ® 200^{26, 27}

| Position (cm⁻¹) | Assignment |
|---|---|
| 3744 | free silanol —Si—OH stretching |
| 3700-3200 | total silanol hydroxyls band; adsorbed H₂O |
| 1980 shoulder, 1870 | overtone structure vibrations of SiO₂ lattice |
| 1625 | SiO₂ lattice vibrations; bending O—H from |
| | adsorbed water |

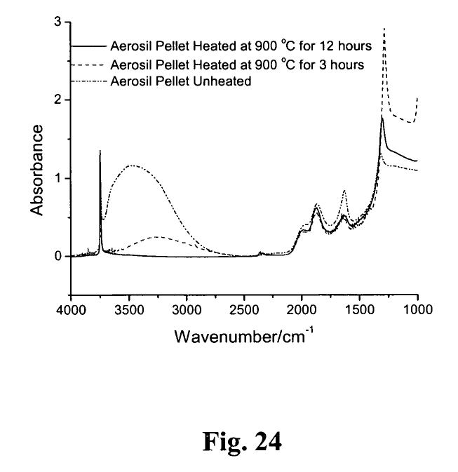

In order to calculate the amount of silane or PMPI per area of Aerosile 200 surface, the total amount of Aerosil® 200 in the IR beam had to be determined. In principle, this amount is obtained by integration of a characteristic silica band whose area is not perturbed by surface modification. Such an internal standard is found in the skeletal modes of the silica^{26,27}, with a particularly suitable region between 1950 cm⁻¹ and 1750 cm⁻¹ (peak center at 1870 cm⁻¹). This region is not affected by removal of surface physisorbed water or condensation of silanols to siloxane bridges at elevated temperatures. FIG. 24 shows infrared absorption spectra of an untreated Aerosil® 200 disk, a disk heated at 900° C. for 3 hours, and a disk heated at 900° C. for 12 hours. Air was used as the background in all cases. The broad peak from 3700 cm⁻¹ to 2750 cm⁻¹ decreases after 3 hours and completely disappears after 12 hours at 900° C. as physisorbed water leaves and surface silanols are eliminated by condensation. The peak at 1631 cm⁻¹ also decreases, consistent with removal of physisorbed water. While the remainder of this peak is attributed to silica lattice vibrations, its sensitivity to moisture content makes it unsuitable for quantitative estimation of the amount of silica present in the beam. Thus the 1950 cm⁻¹ to 1750 cm⁻¹ region is the best candidate^{26,28}.

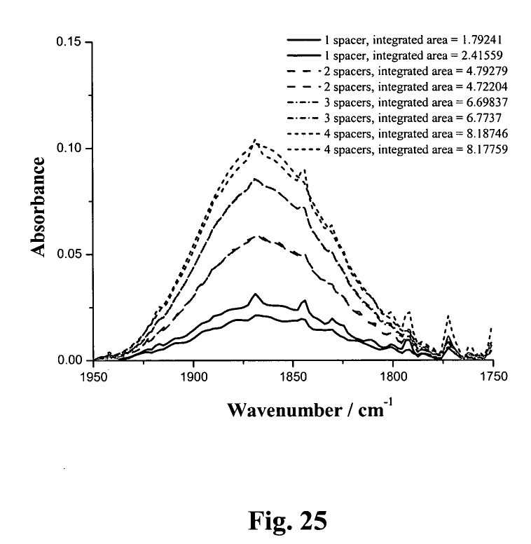

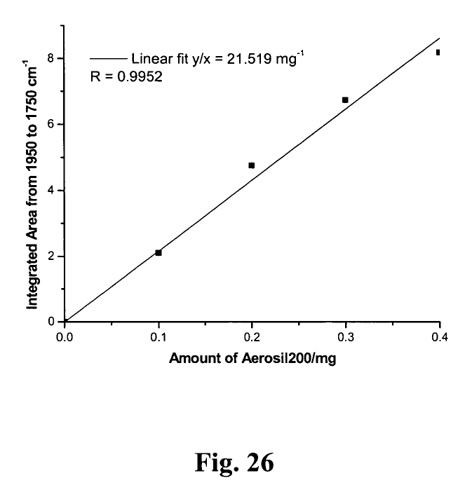

The suitability of the 1950 cm⁻¹ to 1750 cm⁻¹ peak region was verified by quantitatively establishing a linear relationship between the strength of this absorption and the amount of powder present. Different amounts of Aerosil® 200 powder were placed into the beam using stacked O-ring spacers to control the thickness of the powder specimen. Air was used as background, in an otherwise identical sample geometry. As shown in FIG. 25, good reproducibility was observed in independent runs using the same number of stacked spacer. Independently, the mass of silica was measured using a mass balance and multiple spacers; these measurements determined that 0.10 mg of Aerosil® 200 was loaded into the beam per spacer. Using this information, the spectral area between 1950-1750 cm⁻¹ was integrated and plotted vs. mass of silica (FIG. 26). From the slope of the line in FIG. 26, the effective extinction coefficient for Aerosil® 200 powder was found to be 21.5 mg⁻¹. This extinction coefficient was used in subsequent measurements to determine the amount of silica present in the beam from integration of the 1950-1750 cm⁻¹ region.

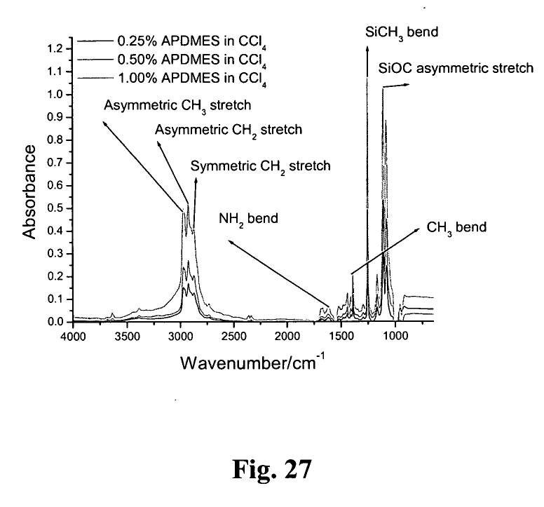

III.2.iii.b. Infrared Spectroscopy of APDMES and APTES Silanes. The commonly accepted infrared spectral assignments for silanes used in this work are listed in Table 3.3²⁷⁻³⁰ for APDMES and Table 3.4²⁸ for APTES. A sample transmission spectrum of APDMES in CCl₄ is depicted in FIG. 27, while FIG. 28 shows a transmission spectrum of APTES on Aerosil® 200.

TABLE 3.3
Principal IR Assignments for APDMES²⁷⁻³⁰:

| Peak Position (cm⁻¹) | Assignment |
|---|---|
| 2967 | antisymmetric CH₃ stretching |
| 2920 | antisymmetric CH₂ stretching |
| 2855 | symmetric CH₂ stretching |
| 1622 | NH₂ bending |
| 1265 | Si—CH₃ bend |
| 1118 | Si—O—C asymmetric stretch |

TABLE 3.4
Principal IR Assignments for APTES²⁹:

| Peak Position | Assignments |
|---|---|
| 3370 | N—H asymmetric stretch |
| 3303 | N—H symmetric stretch |
| 2975 | CH₃ asymmetric stretch |
| 2935 | CH₂ asymmetric stretch |
| 2870 | CH₂ symmetric stretch |
| 1470 | CH₂ bending |

To calculate surface coverage of silane molecules, the extinction coefficient of attached silanes is needed. One could assume that the extinction coefficient in the attached state would be the same as in free solution. However, this is not necessarily the case. One difficulty is due to structural changes in the silanes that arise upon attachment; for example, loss of ethoxy groups when a silane molecule attaches to the silicon oxide surface. Another is that the molecular environment of the surface is different from that in bulk solution, and in general such changes perturb the strength and position of infrared absorption modes.

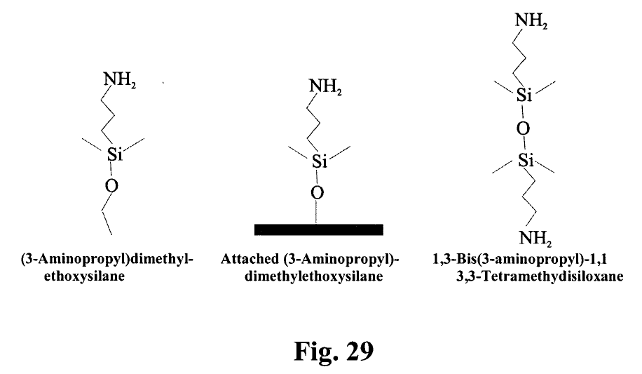

The extinction coefficients of both APDMES and APTES were determined by performing infrared absorbance measurements on samples that were also quantitatively analyzed for coverage of silane using elemental analysis. This allowed direct determination of extinction coefficients of surface-bound silane molecules. For APDMES, a second determination of the extinction coefficient was obtained from absorbance of bulk solutions of 1,3-bis(3-aminopropyl)-1,1,3,3-tetramethydisiloxane (BAPTDS) in CCl₄. BAPTDS is essentially a pair of APDMES molecules condensed together through a siloxane bridge (FIG. 29); therefore, it lacks the ethoxy group of APDMES and was used as a simulant of surface-condensed APDMES silanes. The absorption peaks selected for determination of extinction coefficients of silane molecules (whether on silica or in bulk solution) are the symmetric and unsymmetric CH₃ and CH₂ stretchings. These modes were quantified by integration of silane absorbance spectra from 3025 to 2775 cm⁻¹ (e.g. see FIGS. 27, 28 and 29, and further below).

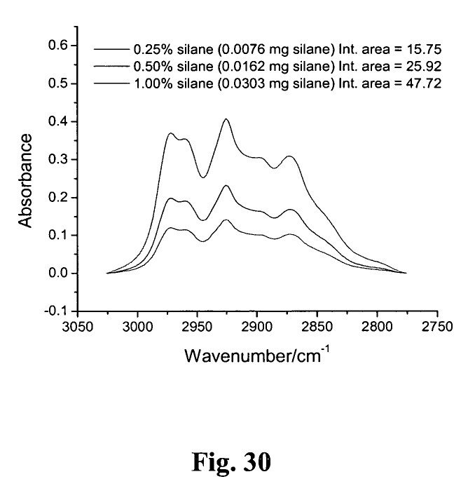

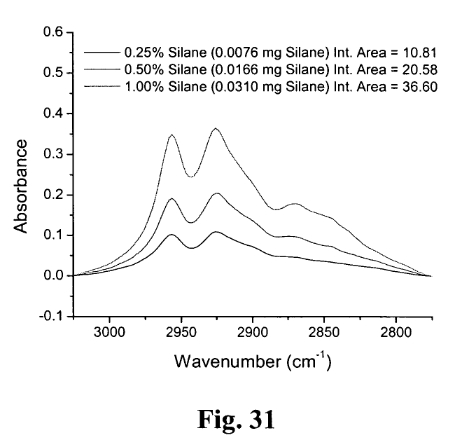

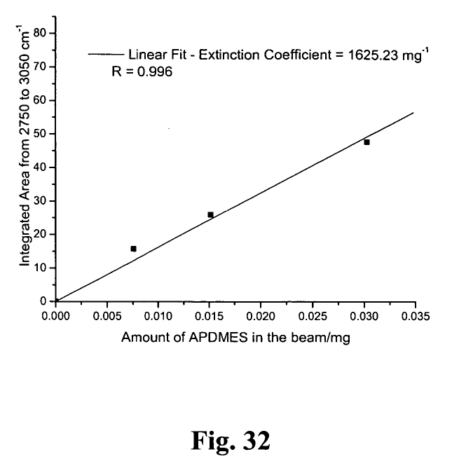

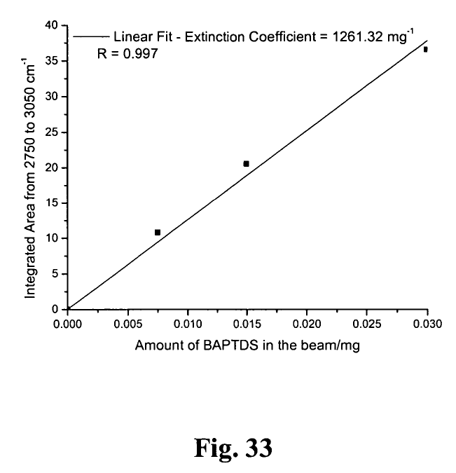

Determination of extinction coefficient for APDMES and BAPTDS in bulk solutions of CCl₄ will be presented first. Solutions of various concentrations of silane in CCl₄ were prepared and their FTIR absorption determined in transmission using a liquid cell (FIGS. 30 and 31). The spectra are qualitatively different, as expected due to presence of the ethoxy group in APDMES. The integrated areas were plotted vs. mass of silane in the infrared beam, using the integrated areas and concentrations (in mg) listed in FIGS. 30 and 31. The resultant plots are depicted in FIGS. 32 (APDMES) and 33 (BAPTDS). The respective slopes yield the extinction coefficients, evaluating to 1625.23 mg⁻¹ for APDMES (FIG. 32), and to 1261.32 mg⁻¹ for BAPTDS (FIG. 33) after dividing by two to account for the fact that BAPTDS corresponds to an APDMES dimer (FIG. 29). As expected, the BAPTDS coefficient is lower because it does not possess contributions due to an ethoxy group. As such, the BAPTDS extinction coefficient is expected to be more representative of that for a surface-bound APDMES molecule (FIG. 29). As discussed next, this expectation was confirmed by comparison with extinction coefficients derived directly from elemental analysis of silane-modified powders.

Elemental analysis (Galbraith Laboratories) was used to determine coverage of APDMES, as well as APTES, residues on modified silica. A series of powders were synthesized with varying coverage of silane and simultaneously analyzed by elemental analysis and transmission infrared spectroscopy. Silane coverage was calculated directly from the elemental analysis data based on increase in carbon content (obtained as a weight percentage) following silanization (see Example 2 for procedure). The increase in carbon content was converted to surface coverage (residues/nm²) assuming full hydrolysis of the silane, leaving 5 carbon atoms per attached APDMES and 3 carbon atoms per attached APTES residue. For APTES, this assumption was consistent with near complete lack of absorptions due to methyl groups in infrared spectra, whose presence would have been manifest if significant fraction of silane ethoxy groups remained. Table 3.5 lists the raw data from elemental analysis (column 2) and the equivalent calculated silane coverages (column 3). The calculations used a specific area of 200 m² per gram of fumed silica.

TABLE 3.5
Elemental Analysis and Infrared Spectral Data Used to
Calibrate the Absorbance of APDMES and APTES on Aerosil ® 200

| | | equiv. Coverage | |
|---|---|---|---|
| | % C (w/w) | silanes per nm^{2a} | spectral ratio^{b} |
| APDMES | | | |
| 1 | 0.88 | 0.44 | 0.59 |
| 2 | 1.31 | 0.66 | 1.26 |
| 3 | 1.82 | 0.91 | 1.85 |
| 4 | 2.11 | 1.06 | 2.21 |
| 5 | 2.29 | 1.15 | 2.62 |
| 6 | 2.31 | 1.16 | 2.48 |
| APTES | | | |
| 1 | 0.71 | 0.59 | 0.56 |
| 2 | 0.91 | 0.76 | 0.93 |
| 3 | 1.02 | 0.85 | 1.10 |
| 4 | 1.19 | 0.99 | 1.27 |
| 5 | 1.34 | 1.12 | 1.60 |
| 6 | 1.38 | 1.15 | 1.63 |
| ^{a}Based on % C from column 2. For calculation assume 1 mg of powder. Mass of silane on powder in grams = (% C × 0.001 g/100). Divide the mass of silane in g by 60.0535 g/mol (=5 carbons) for APDMES and by 36.0321 g/mol (= 3 carbons) for APTES to get moles of silane. Multiply by Avogadro's number to get number of molecules. Multiply 0.001 g powder | | | |
| # by 200 m²/g to get the surface area of powder, and convert to nm² by multiplying by 1 × 10¹⁸. Divide the number of molecules by the surface area of powder in nm² to get the coverage in molecules/nm². | | | |

$$\frac{{\int }_{2775}^{3025} A ( v ) ⅆ v}{{\int }_{1751}^{1951} A ( v ) ⅆ v}$$

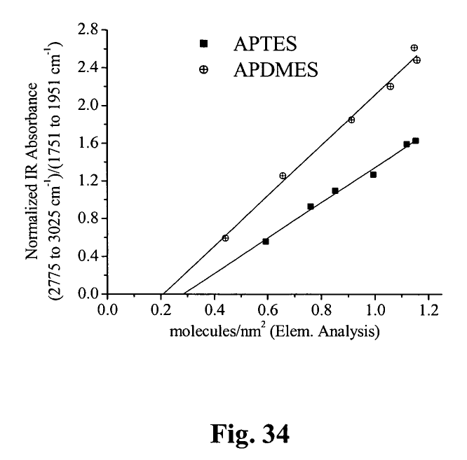

Infrared absorbance spectra, obtained on same silica samples as used for elemental analysis, were integrated from 2775 cm⁻¹ to 3025 cm⁻¹ (after subtraction of a linear baseline between these two limits). As discussed above, this region spans the alkane C—H stretches and provides a measure of the amount of silane present. Next, the integrated value was normalized to the amount of silica in the IR beam. This was accomplished by integrating the silica overtone “internal standard” between 1751 cm⁻¹ and 1951 cm⁻¹ (FIG. 23)—as detailed in section III.2.iii.a this integrated signal is proportional to the amount of silica powder. Dividing the integrated silane absorbance (2775 cm⁻¹ to 3025 cm⁻¹) by that of the silica (1751 cm⁻¹ to 1951 cm⁻¹) yields a ratio proportional to the silane coverage per amount of powder or, equivalently, per unit area of silica surface. This ratio is listed in column 4 of Table 3.5. The proportionality factor needed to convert this ratio into quantitative silane coverage was determined by plotting the ratio against absolute silane coverage obtained from elemental analysis of the same samples. Thus, coverages determined from elemental analysis served to calibrate the infrared absorbance measurements so that, for later samples, silane loadings could be calculated simply from integration of spectral absorption peaks. The resultant plots, shown in FIG. 34, yielded the following calibration relations:

$$APDMES \text{:} Molecules \text{/} {nm}^{2} = \frac{1}{2.7} ( \frac{{\int }_{2775}^{3025} A ( v ) ⅆ v}{{\int }_{1751}^{1951} A ( v ) ⅆ v} ) ( r = 0.996 ) \qquad ( 3.1 )$$

$$APTES \text{:} Molecules \text{/} {nm}^{2} = \frac{1}{1.9} ( \frac{{\int }_{2775}^{3025} A ( v ) ⅆ v}{{\int }_{1751}^{1951} A ( v ) ⅆ v} ) ( r = 0.995 ) \qquad ( 3.2 )$$

where A(v) is the spectral absorbance at wavenumber v (cm⁻¹).

Following features of FIG. 34 are noteworthy. First, a linear relationship applies between APDMES or APTES spectral absorbance and loading, at least for the range of investigated coverages. Second, for APDMES, calculation of the equivalent mass extinction coefficient from the data in Table 3.5 yields 1400 mg⁻¹. As expected, this value is closer to that of BAPTDS (1261 mg⁻¹) than unhydrolyzed APDMES (1625 mg⁻¹) determined from bulk solution measurements (see above). Third, both for APDMES and APTES the calibration does not pass through origin; rather it intersects the x-axis at about 0.25 molecules/nm². This last observation indicates presence of carbon even in unmodified silica, and is attributed to adventitious surface contamination and to carbon within the silica particles themselves. Both the APTES and APDMES samples possessed similar amounts of the carbon “background”, suggesting that the silanization protocol was not successful in dislodging such contaminants regardless of whether the incoming silane was capable of just a single (APDMES) or multiple (APTES) bond formation to the silica surface. Notably, the carbon background was subtracted in deriving relations 3.1 and 3.2; thus, these relations yield silane coverages as if the fits in FIG. 34 passed through the origin.

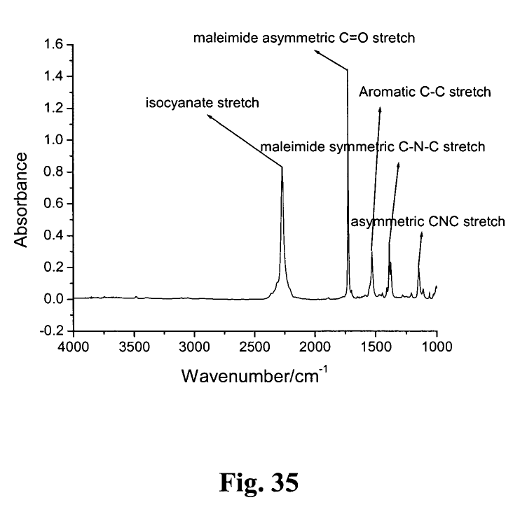

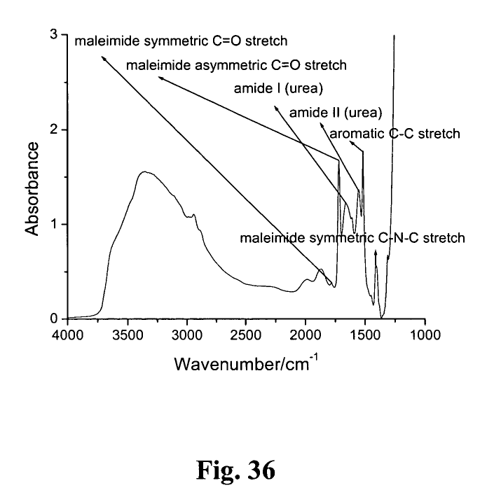

III.2.iii.c. Infrared Spectroscopy of PMPI. Tentative infrared spectral assignments for the crosslinker PMPI in bulk CCl₄ solution and after attachment to an APTES silanized powder are listed in Tables 3.6a and 3.6b, respectively, following literature attributions for similar molecules^{27,31,32}. A bulk transmission spectrum of PMPI in CCl₄ is depicted in FIG. 35, while FIG. 36 shows a transmission spectrum from Aerosil® 200 powder that was first modified with APTES and then reacted with PMPI.

TABLE 3.6a
Principal IR Assignments for PMPI in CCl₄^{27, 31, 32}:

| Peak Position | Assignments |
|---|---|
| 2269 | isocyanate stretch |
| 1725 | maleimide asymmetric C═O stretch³² |
| 1531 | aromatic C—C stretch³² |
| 1405 | maleimide symmetric C—N—C stretch³² |
| 1150 | asymmetric CNC stretch |

TABLE 3.6b
Principal IR Assignments for PMPI on Aerosil ® 200 powder^{31, 32}:

| Peak Position | Assignments |
|---|---|
| 1776 w | maleimide symmetric C═O stretch³² |
| 1717 s | maleimide asymmetric C═O stretch³² |
| 1658 s | amide I (urea)³¹ |
| 1550 s | amide II (urea)³¹ |
| 1513 s | aromatic C—C stretch³² |
| 1405 s | maleimide symmetric C—N—C stretch³² |

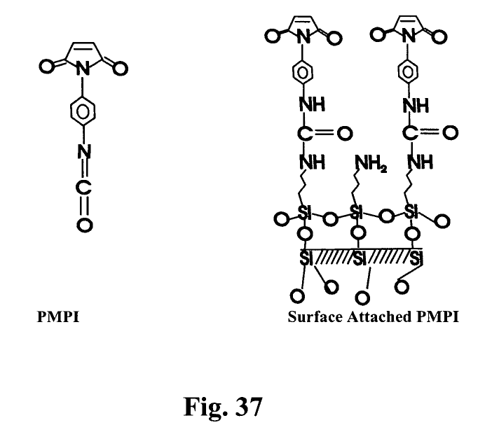

As expected, after reaction of PMPI with APTES the isocyanate stretch disappears while pronounced absorption peaks associated with formation of a urea linkage between PMPI and APTES appear. These changes reflect reaction of PMPI's isocyanate (N═C═O) groups through nucleophilic addition at the carbonyl carbon with the primary amines of APTES, forming urea (—HN—CO—NH—) bonds (FIG. 37).

The absorbance peak at 1405 cm⁻¹, attributed to the symmetric C—N—C stretch of the PMPI maleimide moiety, was chosen for quantification of PMPI surface coverages. This absorbance mode was selected based on criteria of minimal overlap with other spectral features. The strength of this absorbance was quantitatively related to surface coverage of PMPI (residues/nm²) using elemental analysis to establish absolute PMPI loadings, in a manner analogous to that employed earlier to derive relations 3.1 and 3.2 for the case of silane coverages.

Aerosil® silica was first modified with APTES to a coverage of about 0.55 APTES/nm² (calculated from IR spectra via relation 3.2, averaged over all samples). The modified powder was then split into aliquots, and each aliquot was reacted (2 hrs, 22° C.) with a different concentration solution of PMPI in acetonitrile, ranging from 0 to 0.36% w/w (column 1 in Table 3.7). The product of each reaction was analyzed both by elemental analysis and by transmission IR spectroscopy. The elemental analysis % C (w/w) values of PMPI modified powders are listed in column 2 of Table 3.7. % C values measured on unreacted powders #8 and 9, which include contributions due to APTES as well as carbon background (e.g. carbon within the silica particles and from adsorbed organics such as due to remnants of solvent) were averaged and subtracted from powders reacted with PMPI to afford increase in carbon content attributed solely to reaction with PMPI. The % C values were converted to coverage of PMPI, taking 11 carbon atoms per PMPI residue (column 3, Table 3.7). Results from IR analysis of same powders are depicted in column 4 of Table 3.7. The integrated area from 1363 cm⁻¹ to 1430 cm⁻¹ (delimiting the 1405 cm⁻¹ peak) was normalized to the amount of powder as metered by the integrated area from 1751 cm⁻¹ to 1951 cm⁻¹ to yield the spectral ratio.

TABLE 3.7
Elemental Analysis and Infrared Spectral Data Used to
Calibrate the Absorbance of PMPI on Aerosil ® 200

| PMPI | | | |
|---|---|---|---|
| conc. (w/w)^{a} | % C (w/w)^{b} | coverage PMPI per nm^{2c} | spectral ratio^{d} |
| 0.00013 | 1.72 | 0.02164 | 0.01481 |
| 0.00026 | 1.80 | 0.03987 | 0.0774 |
| 0.00077 | 2.58 | 0.21757 | 0.368 |
| 00.0013 | 3.13 | 0.34288 | 0.678 |
| 0.0018 | 3.62 | 0.45451 | 0.91 |
| 0.0028 | 4.16 | 0.57754 | 1.11 |
| 0.0036 | 4.46 | 0.64589 | 1.39 |
| 0 | 1.56 | −0.01481 | −0.09 |
| 0 | 1.69 | 0.01481 | −0.07 |
| ^{a}Concentration of PMPI in acetonitrile used for reaction with APTES modified Aerosil ®. | | | |
| ^{b}Mass percentage of carbon in PMPI modified powder samples determined by elemental analysis. | | | |
| ^{c}Based on % C from column 2. First, background carbon, given by average of last two entries (=1.625), was subtracted. For calculation assume 1 mg of powder. Mass of PMPI on powder in grams = (% C × 0.001 g/100). Divide the mass of PMPI in g by 132.12 g/mol (=11 carbons) to get moles of PMPI. Multiply by Avogadro's number to get number of | | | |
| # molecules. Multiply 0.001 g powder by 200 m²/g to get the surface area of powder, and convert to nm² by multiplying by 1 × 10¹⁸. Divide the number of molecules by the surface area of powder in nm² to get the coverage in molecules/nm² (column 3). | | | |

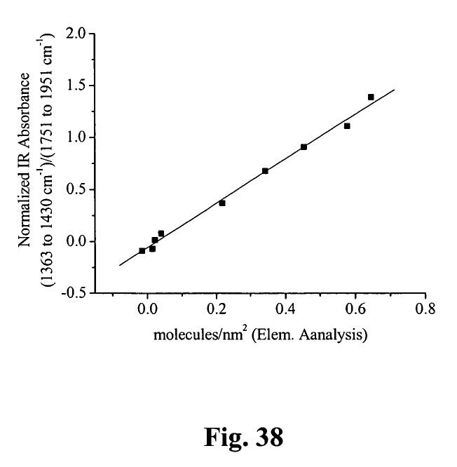

A plot of the spectral ratio vs. surface coverage of PMPI is shown in FIG. 38. From the figure, the following linear calibration was derived:

$$\frac{{\int }_{1363}^{1430} A ( v ) ⅆ v}{{\int }_{1751}^{1951} A ( v ) ⅆ v}$$

where A(v) is the absorption of the spectrum, v is the wavenumber in cm⁻¹.

III.2.iii.d. Infrared Spectroscopy of MBS. Spectral assignments for the

$$PMPI \text{:} Molecules \text{/} {nm}^{2} = \frac{1}{2.1} ( \frac{{\int }_{1363}^{1430} A ( v ) ⅆ v}{{\int }_{1751}^{ 1951} A ( v ) ⅆ v} ) ( r = 0.997 ) \qquad ( 3.3 )$$

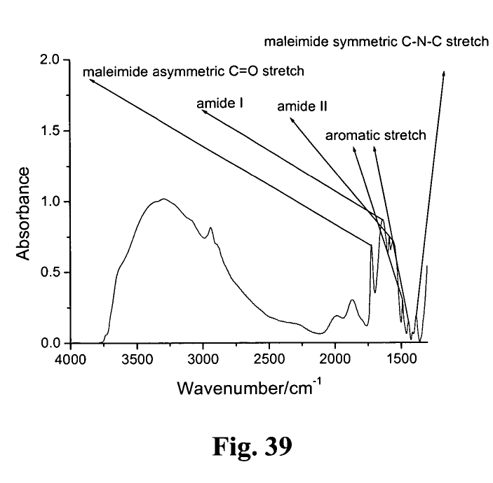

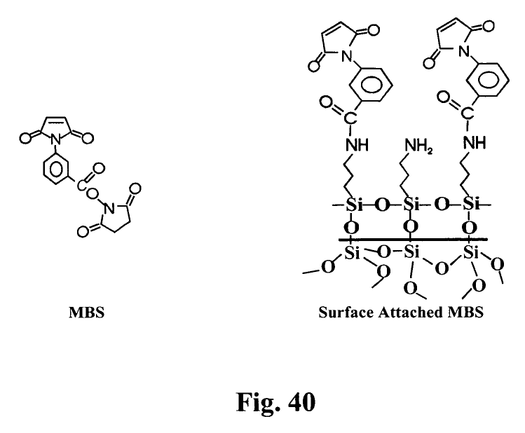

crosslinker MBS after attachment to APTES silanized powder are listed in Table 3.8, following literature attributions^{27,32}. FIG. 39 shows a transmission spectrum obtained from an Aerosil® 200 sample after it was first functionalized with APTES and then with MBS. The Amide I and II absorptions confirm reaction of crosslinker with APTES amines, forming an amide linkage. FIG. 40 depicts the expected attachment geometry of MBS on APTES-modified silica.

TABLE 3.8
Principal IR Assignments for MBS on APTES^{27,32}:

| Peak Position (cm⁻¹) | Assignments |
|---|---|
| 1721 s | maleimide asymmetric C═O stretch³² |
| 1636 s | amide I |
| 1550 s | amide II |
| 1488 m | aromatic stretch²⁷ |
| 1438 m | aromatic stretch²⁷ |
| 1388 m | maleimide symmetric C—N—C stretch³² |

^{a}s = strong; m = medium; w = weak

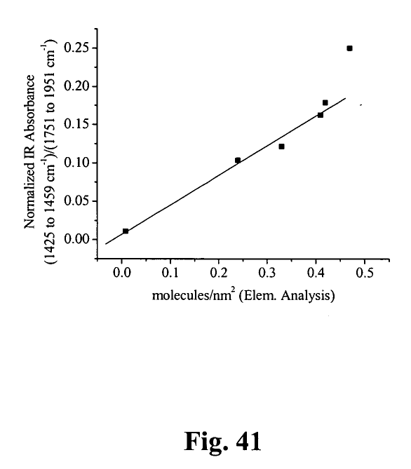

MBS modified powders were analyzed by elemental analysis and transmission IR spectroscopy, using procedures analogous to those for PMPI modified samples (section III.2.iii.c). The elemental analysis and corresponding IR data are shown in table 3.9, and the calibration plot in FIG. 41. An aromatic stretch at 1438 cm⁻¹ was selected as the calibration peak. The following linear fit was derived from the data for calculating MBS coverages from IR spectra:

$$MBS \text{:} Molecules \text{/} {nm}^{2} = \frac{1}{0.38} ( \frac{{\int }_{1425}^{1459} A ( v ) ⅆ v}{{\int }_{1751}^{ 1951} A ( v ) ⅆ v} ) ( r = 0.992 ) \qquad ( 3.4 )$$

TABLE 3.9
Elemen tal Analysis and Ifrared Spectral Data Used to
Calibrate the Absorbance of MBS on Aerosil ® 200

| | | coverage | |
|---|---|---|---|
| MBS conc. (w/w)^{a} | % C (w/w)^{b} | MBS per nm^{2c} | spectral ratio^{d} |
| 0.00015 | 1.7 | 0.008 | 0.01087 |
| 0.0015 | 2.74 | 0.24 | 0.10334 |
| 0.0038 | 3.1 | 0.33 | 0.12148 |
| 0.0068 | 3.46 | 0.41 | 0.16226 |
| 0.011 | 3.53 | 0.42 | 0.17835 |
| 0.021 | 3.72 | 0.47 | 0.25 |
| 0 | 1.53 | | |
| 0 | 1.8 | | |

^{a}Concentration of MBS on acetonitrile used for reaction with APTES
modified Aerosil ®.
^{b}Mass percentage of carbon in MBS modified powder samples determined
by elemental analysis.
^{c}Based on % C from column 2. First, background carbon, given by
average of last two entries (=1.665), was subtracted. For calculation
assume 1 mg of powder. Mass of MBS on powder in grams = (% C ×
0.001 g/100). Divide the mass of MBS in g by 132.12 g/mol (=11 carbons)
to get moles of MBS. Multiply by Avogadro's number to get number of
molecules. Multiply 0.001 g powder by 200 m²/g to get the surface
area of powder, and convert to nm² by multiplying by 1 × 10¹⁸.
Divide the number of molecules by the surface area of powder in nm² to
get the coverage in molecules/nm² (column 3).
^{d}Spectral absorbance of MBS normalized to amount of powder:
${\int }_{1425}^{1459} A ( v ) ⅆ v / {\int }_{1751}^{1951} A ( v ) ⅆ v$
where A is absorbance and ν is wavenumber (cm⁻¹).

From FIG. 41, it is evident that the linear fit applies to surface coverages up to about 0.45 molecules/nm². Above this value, the aromatic stretch mode of MBS appears sensitive to the MBS coverage in a nonlinear manner, with the consequence of being enhanced. Therefore, use of relation 3.4 to estimate surface coverages from IR data when actual coverage exceeds 0.45 MBS/nm² is expected to overestimate the true number of MBS molecules per area.

III.2.iv. Analysis of Maleimide Activity of Modified Aerosil® Supports. The surface density of active maleimide groups on silica powder following immobilization of PMPI or MBS crosslinker was determined using Ellman's assay^{33,34}. The protocol has been previously described in Example 2, and follows instructions from the provider (Pierce Biotechnology). Briefly, L-cysteine was used to titrate 1 mg aliquots of functionalized powder, and the decrease in bulk concentration of L-cysteine due to reaction with surface maleimides was monitored spectrophotometrically using Ellman's reagent. Assuming a 1:1 stoichiometry of reaction between L-cysteine and surface maleimides, the surface coverage of active and accessible maleimide groups was estimated. APTES-modified silica without crosslinker, for which no active maleimides should be observed, served as a control.

III.2.v. Immobilization of Oligonucleotides on Glass Slides. Glass slides (Fisherfinest Premium Microscope Slides) were sonicated 10 minutes each in chloroform, ethanol, water, and dried. The slides were modified with APTES and PMPI crosslinker prior to DNA immobilization. Modification with APTES was performed under 2.8 mM solution in anhydrous toluene for 30 minutes at room temperature while stirring. Slides were washed twice with toluene, once with deionized water, once with acetonitrile, followed by drying overnight at 100° C. Duration of each wash was 10 minutes. APTES-coated slides were exposed to 0.5 mg/ml PMPI in anhydrous acetonitrile for 2 hrs at room temperature with gentle stirring. After attachment of PMPI, slides were washed once with fresh acetonitrile and once with PBS, at 10 minutes per wash (PBS: 10 mM sodium phosphate, 1 M NaCl, pH 7). Disulfide terminated PSH oligonucleotides (Table 3.1) were cleaved with DTT in PBS (200 fold excess DTT over disulfide; 1 hr) to liberate the thiol groups and purified on PD-10 columns (Amersham Biosciences). Solutions of freshly prepared PSH oligonucleotides were used immediately to functionalize PMPI-derivatized slides (0.1 uM to 1 uM oligonucleotide in PBS; 2 hrs; room temperature). For this step a 1.5 ml chamber, closed to the ambient environment, was created by sandwiching a silicone O-ring between two identically functionalized glass slides. DNA solutions were introduced to the chamber via a syringe and needle inserted through the O-ring, which thus doubled as a septum. The unmodified P oligonucleotides were attached in an identical fashion except for the omission of the DTT cleavage step. Additional procedural details appear in Appendix III.5.i.

Coverage of immobilized oligonucleotides was determined from change in bulk concentration of PSH or P oligonucleotide before and post attachment. Concentrations were measured using OliGreen® stain from Molecular Probes according to manufacturer's instructions (Example 2), using an SLM 8000 fluorometer.

III.2.vi. Hybridizations. Surfaces derivatized with PSH oligonucleotides were exposed to 0.1 μM solutions of fully complementary TC strands and, as a control for sequence specificity of hybridization, to noncomplementary TNC target sequences in PBS. Hybridizations were allowed to proceed for 30 hrs at room temperature. The TC and TNC strands were labeled with fluorescein so as to allow quantification of hybridization extents from decrease in bulk solution fluorescence. As hybridization yields were determined from changes in fluorescence of bulk solutions, washing or other post-processing of the surface, which perturbs hybridization equilibrium, did not affect the measured values. In addition, qualitative confirmation of hybridization was obtained by laser scanning confocal microscopy (Olympus IX-70 microscope equipped with an Ar/Kr laser), for which hybridized slides were washed twice with PBS for 2 minutes each wash and dried with compressed nitrogen prior to scanning. Fluorescence confocal microscopy proved not suitable for quantitative work, however, due to fluorescence quenching at high extents of hybridization on account of proximity between neighboring fluorescent tags on the surface³⁵.

III.3. Results and Discussion of Example 3.

III.3.i. Silanization. The selection of APTES and APDMES was motivated by unique properties of aminosilane reagents. It was desired to employ anhydrous conditions to minimize hydrolysis and oligomerization of the silane in bulk solution to improve reproducibility of the resultant layer, with the ideal outcome consisting of a monolayer of molecules each of which is covalently bound to the silica support via one or more siloxane bridges (FIG. 22). In the absence of water to hydrolyze the alkoxy groups and generate silane silanols, formation of siloxane bonds between alkoxysilanes and surface silanols requires an amine catalyst³⁶. Since aminosilanes such as APTES and APDMES contain the catalyzing amine functionality in the same molecule, a significant fraction of adsorbed silanes attaches covalently to the solid support even under anhydrous conditions and at ambient temperature^{30,37}. In addition to formation of siloxane bridges, aminosilanes interact with surface silanols through the amine terminus via hydrogen bonding and ionic interactions³⁸⁻⁴⁰. In general, the conformation and connectivity of bound aminosilanes, to the surface and to each other (as with APTES when intersilane siloxane bonds may also form), is expected to be complex.

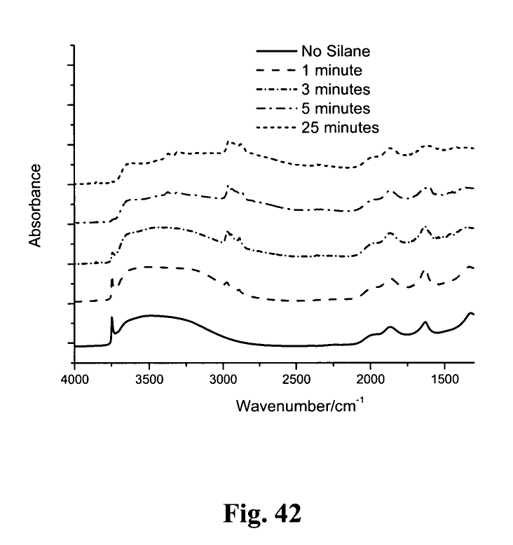

III.3.i.a. Silanization of Aerosil® 200 with APDMES. In order to visualize the progression of IR spectral changes that occur as silanization progresses, the silanization of APDMES with time has been measured. 25 mg of Aerosil® 200 powder were pressed at 6 kbar for 10 minutes to make self-supporting disks. Each disk was put into a 3% v/v APDMES solution in toluene for a different length of time. After washing and drying of the disks, FTIR transmission scans were performed with transmission through air used as background (FIG. 42). The principal changes evidenced are the progressive appearance of the APDMES CH₂ and CH₃ stretches between 2800 and 3000 cm⁻¹, and the APDMES amine NH stretches near 3300 cm⁻¹, and the disappearance of the sharp band due to free silica silanols at 3744 cm⁻¹. These changes are reflective of the covalent immobilization of APDMES to the silica.

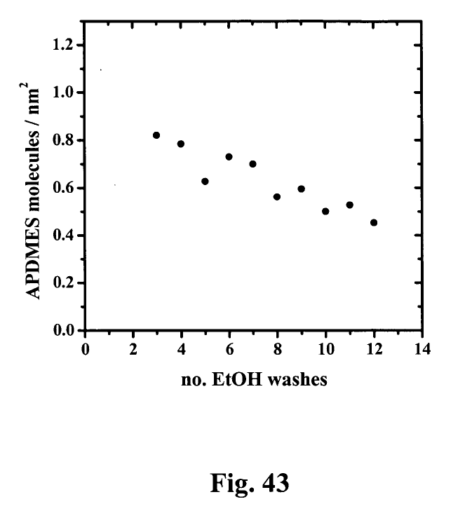

The stability of APDMES attachment as evaluated in a separate set of experimental trials. 300 mg of APDMES Aerosil® 200 powder was prepared in ethanol in a 15 ml plastic centrifuge tube (3% v/v APDMES in ethanol, 40 minute reaction time). The 100° C. overnight curing/drying step was not performed in order to test silane stability immediately post attachment. The powder was centrifuged and the supernatant was discarded. A 20 mg aliquot of sample was put aside, and 5 ml of fresh ethanol were added to the remaining powder, dispersed for 2 minutes under agitation, centrifuged, and decanted. A second 20 mg aliquot was put aside, and the above procedure was repeated for 11 more times to generate a set of samples subjected from 1 to 12 two-minute ethanol washes. Finally, all samples were dried together overnight in the oven at 50° C. IR measurements were performed on the dried samples to determine coverage of remaining APDMES after each wash. The results are displayed in FIG. 43.

As evident from FIG. 43, the amount of APDMES continually decreases with each additional ethanol wash, suggesting a lack of stability to alcoholysis of the silane-to-silica siloxane linkage. This observation prompted the replacement of APDMES as the aminosilane of choice with APTES. Because APTES possesses three ethoxy groups on its silicon atom, compared to one for APDMES, APTES is capable of forging multiple siloxane bonds to the silica. Indeed, APTES has been shown to possess significantly improved stability over APDMES in similar studies⁴¹.

III.3.i.b. Silanization with APTES. FIG. 4 shows APTES coverage (molecules/nm²) on Aerosil® 200 silica after a 30 minute exposure to solutions of variable silane concentration in anhydrous toluene, followed by washing and drying as described in section III.2.ii. Short reaction times were employed to realize submonolayer coverages of APTES 42 The x-axis represents the number of silane molecules present in the toluene solution per nm² of available silica surface. For the employed protocol, coverage approached a plateau close to 0.9 APTES molecules/nm² when this bulk to surface excess exceeded 2.5 molecules/nm², corresponding to a concentration of ˜0.4% w/w.

Most literature studies of APTES reaction with silica under anhydrous conditions report chemical (i.e. covalently attached) loadings of 2 molecules/nm² or greater^{38,39,43}, well above the value of 0.9 molecules/nm² reported in FIG. 6. However, Aerosil® 200 has fewer surface silanols than the fully hydroxylated silica typically used, about 2.8 silanols/nm^{2 44} compared to 4.6 silanols/nm^{2 45}. For partially dehydroxylated silica surfaces with comparable silanol densities to Aerosil® 200, Vrancken et al reported a coverage of about 1.7 APTES/nm² after a 2 hr deposition from 1% toluene solutions and curing for 20 hrs under vacuum at 423° K⁴⁶. In the present study, only a 30 minute deposition time was used and the samples were not cured but washed immediately, so that only those silane molecules that reacted covalently during the relatively short deposition step were expected to remain. The lower APTES loadings are attributed to the above differences in preparation protocol. Subsequent studies of immobilization of PMPI and MBS crosslinkers employed silica with the plateau, submonolayer coverage of ˜0.8 to 0.9 APTES molecules/nm².

III.3.ii. Modification of APTES-silica with PMPI and MBS Crosslinkers. The heterobifunctional crosslinker PMPI was originally developed for preparing protein conjugates⁴⁷. The isocyanate moiety in this linker (FIG. 22) is highly reactive toward alcohol or amine groups, forming stable carbamate or urea bonds respectively, whereas the linker's maleimide moiety reacts with thiols to create thioether links. The steric accessibility of the isocyanate group also renders PMPI ideal for reacting in the crowded environment of a surface, where saturation of reactive surface groups is desired. Notwithstanding these potential advantages, PMPI does not appear to have been used previously for surface modification.

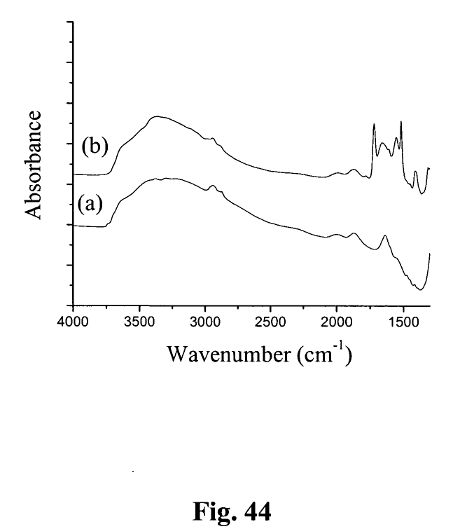

As the isocyanate moiety is unstable in water, reaction of PMPI with APTES-modified silica was carried out in anhydrous acetonitrile. In infrared spectra, PMPI attachment was confirmed by disappearance of the APTES amine stretches at 3303 and 3370 cm⁻¹ as these amines reacted to form the urea linkage, and the appearance of several strongly absorbing modes in the 1400-1800 cm⁻¹ region associated with PMPI (FIG. 44; Table III.6b). In control experiments, no evidence was found of PMPI attachment to neat silica that had not been modified with APTES.

FIG. 5 plots linker coverage and resultant maleimide activity of the surface as a function of the bulk concentration of linker applied. The x-axis represents concentration of PMPI molecules in bulk solution, expressed as number of molecules added per nm² of silica surface. A unit of one on this axis corresponds to a concentration of 13 mM PMPI in acetonitrile; thus, the concentration range investigated was 0.62 mM to 67 mM. PMPI coverage was determined from infrared absorption spectra via equation 3.3, while maleimide activity was obtained from Ellman's analysis as described earlier. A dotted line was drawn in FIG. 5 to indicate surface coverage of APTES molecules on the silica, which was 0.90 molecules/nm².

Surprisingly, FIG. 5 shows the PMPI coverage (filled circles) to rise fairly quickly to surpass that of APTES, appearing to exceed it by as much as 50% at highest concentrations investigated. Evidently, achievement of a 1:1 stoichiometry between PMPI molecules and APTES silanes is not limiting; in other words, more linker molecules may attach than there are available amines. Analysis via Ellman's assay showed the active maleimide coverage to approach, but not to exceed, that of APTES (open circles in FIG. 5). As there are fewer active maleimides than PMPI residues, a fraction of the maleimides are either inaccessible to L-cysteine or have become inactive.

The above observations may be interpreted as follows. Inevitably, trace water will be present in the reaction mixture, with primary source presumably physisorbed moisture associated with the high surface area of the fumed silica. The isocyanate group of PMPI readily reacts with water to form an unstable carbamic acid which decomposes to evolve CO₂ and to leave an amine group in place of the isocyanate⁴⁸. The generated amine reacts with a second PMPI molecule to form the bismaleimide compound N,N′-bis(p-maleimidophenyl)urea⁴⁷, which attaches to surface amines via Michael addition to one of the maleimide termini (FIG. 6). Maleimide-amine additions are well known and used in a variety of applications, e.g. polymer resins⁴⁹. In the above described scenario, two PMPI residues are introduced per surface silane. The amines of hydrolyzed PMPI molecules also react with maleimides already on the surface, opening a second avenue for vertical stacking of PMPI residues. The number of active maleimides per area, however, cannot exceed the number of APTES molecules available, in agreement with the data of FIG. 5. To summarize, the experimentally observed difference between coverage of PMPI residues and active maleimides is believed to reflect a distribution of addition products on the surface, encompassing both single and multiply stacked PMPI adducts.

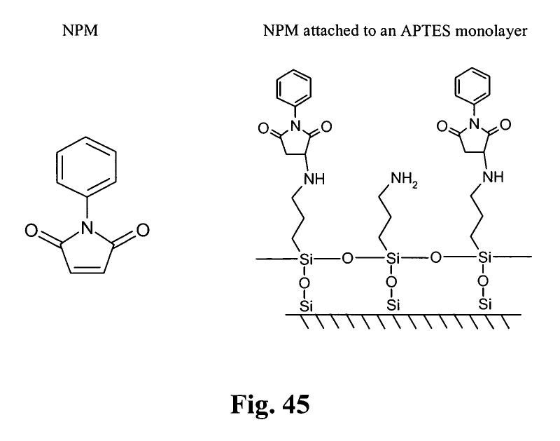

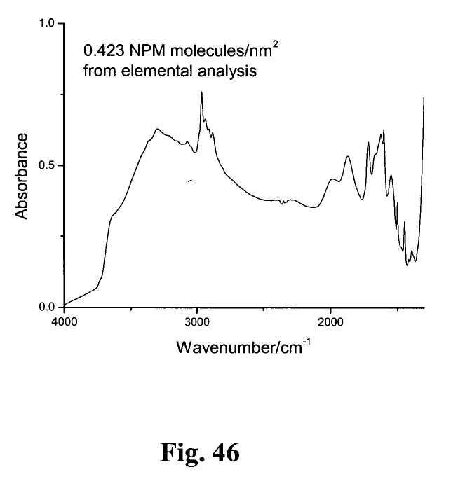

The potential for Michael addition between APTES derivatized silica and maleimide groups was independently confirmed using N-phenylmaleimide (NPM). NPM possesses a single maleimide group, and no other sites through which addition to an amine might be postulated to take place (FIG. 45 left). A two-hour reaction with 33 mM NPM in acetonitrile at room temperature resulted in a coverage of 0.34 NPM molecules/nm², as determined by elemental analysis. However, only about 0.05 maleimides/nm² remained active according to Ellman's assay, clearly indicating that NPM attached via the maleimide moiety (FIG. 45 right). FIG. 46 shows an IR spectrum of Aerosil® 200 reacted with APTES and then with NPM. Notably, as expected based on the postulated addition mechanism in FIG. 45, the APTES amine NH stretches at ˜3350 cm⁻¹ are absent while a number of bands due to NPM appear in the 1400 to 1800 cm⁻¹ region.

Qualitative confirmation of maleimide activity on NPM and PMPI modified powders could be obtained by visual inspection. Both NPM and PMPI are yellow in color. Silica modified with NPM appeared colorless. The lack of color was attributed to loss of conjugation of NPM when it reacted with silane amines through its maleimide double bond. In contrast, silica modified with PMPI was yellow because attachment occurred via the PMPI isocyanate end, leaving the maleimide double bond intact.

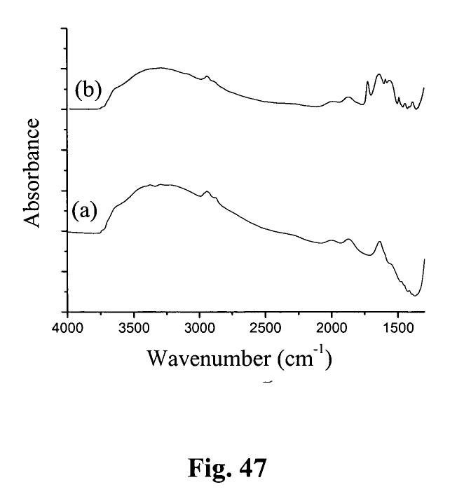

Heterobifunctional linkers designed to crosslink amine and thiol moieties most often rely on NHS-ester as the amine-reactive site⁵⁰. Therefore, as a comparison to PMPI, the NHS-ester linker MBS was also investigated. In infrared spectra, attachment of MBS was identified by disappearance of APTES amine N—H stretches and the appearance of modes attributed to MBS and the formation of an amide linkage to surface amines (Table 3.8, FIG. 47).

FIG. 47 plots MBS coverage and maleimide activity as a function of bulk concentration of linker used. The highest concentration, corresponding to a solution to surface excess of 5.58 molecules/nm², represents 53 mM. No attachment of crosslinker was observed in control experiments employing silica powder without APTES. The trends in FIG. 47 are strikingly different from those for PMPI (FIG. 5). The linker coverage reaches at most 60% that of the silane. Therefore, saturation of the aminosilane layer with linker does not occur. Second, the activity of maleimide groups is very low. These results indicate a failure of the MBS linker to generate significant enrichment of maleimide groups on the APTES modified silica.

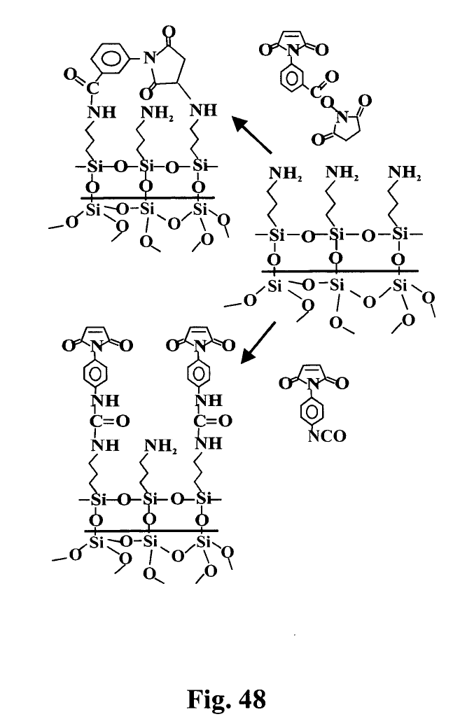

The evident lack of active maleimides may be understood as follows. If surface amines are not consumed quickly by reacting with MBS NHS-esters to form amide bonds, then an MBS molecule immobilized via its NHS-ester site will be surrounded by reactive amines. The maleimide of the linker then becomes susceptible to deactivation by nucleophilic attack by one of the neighboring amines. This sequence of events would leave a majority of linkers attached via both ends (FIG. 48), and therefore with low remnant maleimide activity in agreement with the data of FIG. 7. Moreover, since two surface amines are consumed per doubly-bonded linker, linker coverage will not be able to approach that of APTES. This expectation is consistent with the MBS under-saturation of the APTES coverage evident in FIG. 7. At comparable solution to surface excess, such undersaturation is not observed in the case of PMPI (FIG. 5). Presumably, the enhanced reactivity of the PMPI isocyanate group, as opposed to the NHS-ester of MBS, allows it to more quickly and fully react with surface amines and thus prevent their subsequent cross reactions with maleimide moieties (FIG. 48).

Experiments were also performed with the water-soluble linker sulfo-GMBS, immobilized to APTES functionalized silica from 10 mM, pH 8 potassium phosphate buffer. As with MBS, the reactive sites on sulfo-GMBS are an NHS-ester and a maleimide. No detectable maleimide activity was realized when sulfo-GMBS was immobilized using a solution to surface excess of 1 molecule/nm² (4.65 mM). However, significant activity of about 0.4 maleimides/nm² was obtained for a solution to surface excess of 10 molecules/nm² (46.5 mM). Thus, by raising the concentration of linker in bulk solution, it was possible to saturate the surface amines sufficiently rapidly to retain a fraction of active maleimide groups. Recovery of maleimide activity at higher linker concentrations is also expected with MBS, though this was not realized under the investigated conditions (FIG. 7).

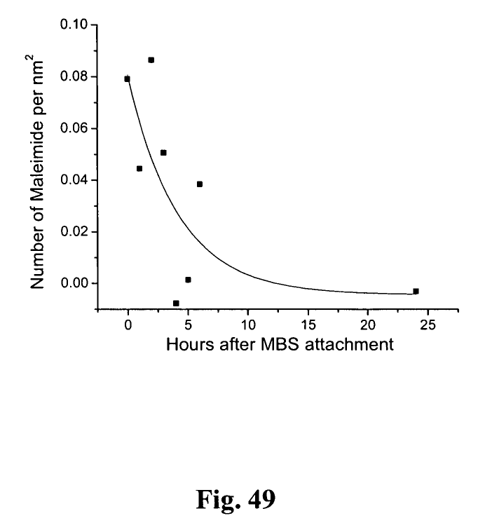

Assays for maleimide activity were carried out after drying of modified silica powder, postponing analysis by a day. In order to determine maleimide activity immediately after preparation, PMPI and MBS modified silica was also characterized while still wet with acetonitrile solvent. For these measurements, a small amount (approx. 0.7% v/v) of acetonitrile was present during the titration with L-cysteine. Linker immobilization was carried out from 50 mM solutions in acetonitrile for 1 hr. The PMPI sample possessed 0.8 APTES per nm² and 1.1 PMPI molecules per nm². With these coverages, there should be very few unreacted amines left on the surface. The density of active maleimides immediately post attachment was 0.75 per nm², while after 23 hours the density decreased to 0.70 per nm². Incidentally, this slight decrease indicates that deactivation of maleimides by ambient contaminants during laboratory storage, if present, was minimal. The MBS sample, with 0.9 APTES per nm² and 0.45 MBS per nm², had ˜0.1 active maleimides per nm² immediately after attachment, which decreased to undetectable levels (less than 0.05 per nm²) after 5 hrs (FIG. 49). Therefore, most MBS maleimides were deactivated during the relatively brief reaction with the surface rather than during subsequent drying of the powder.

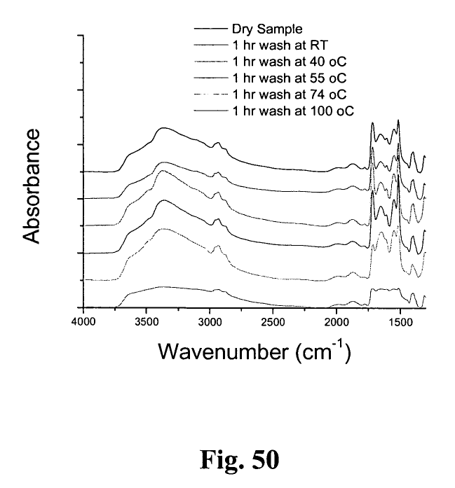

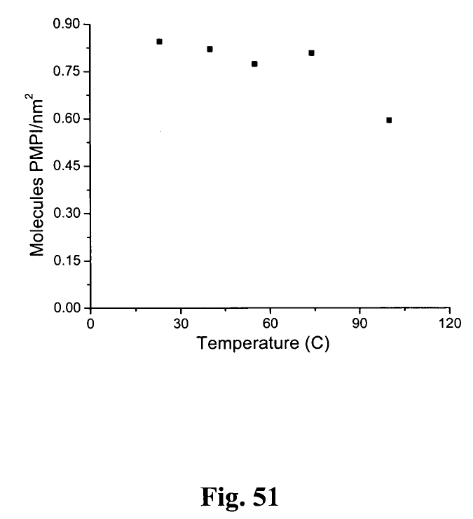

An important consideration for use of modified silica surfaces in biodiagnostics is stability of such surfaces to a range of environmental conditions. An evaluation of thermal stability, in terms of retention of PMPI residues on the silica surface, has been carried out. Aliquots of PMPI modified powders were dispersed in deionized water for 1 hour at temperatures from 20° C. to 100° C. IR spectra of the powders after drying are plotted in FIG. 50. The remaining coverage of PMPI residues on the surface after each wash is depicted in FIG. 51. These results suggest that attachment of PMPI residues to the silica surface is fairly stable to temperatures less than about 70° C., but that noticeable loss occurs at higher temperatures. Importantly, these experiments did not assay for activity of maleimide groups after the temperature wash. Maleimides are themselves susceptible to hydrolysis and may become deactivated (spectral changes are evident in FIG. 50 which at present are not fully understood).

III.3.iii. DNA Immobilization and Hybridization. Planar microscope slides, rather than fumed silica, were used to demonstrate immobilization of DNA oligonucleotides and their hybridization. This change of solid support was required as the high surface area of fumed silica would require prohibitive quantities of DNA. Attachment of the 20mer oligonucleotide PSH (Table 3.1) was performed from 1 uM or 0.1 uM solutions in PBS for 15 hours at room temperature. Approximately 1.5 ml of solution was injected into a chamber created by a silicone O-ring between two PMPI-activated glass slides of known area. PMPI rather than MBS activation was used based on the results of the prior section, which showed PMPI to be more effective in creating maleimide enriched supports.

Because oligonucleotide immobilization was quantified by fluorometric quantification of decrease in DNA strand concentration in bulk solution, potential loss of accuracy could result from adsorption of oligonucleotides to the internal surfaces of the O-ring. Separate control experiments confirmed such losses to be less than 5%. As an additional control to check that attachment occurred site specifically via the thiol moiety, the P oligonucleotide (Table 3.1) with the same sequence as PSH but lacking the 3′ thiol modification was immobilized following the same protocol.

FIG. 8 displays the results of these experiments. For the PSH oligonucleotide, the higher 1 uM concentration yielded a dense DNA monolayer, with 2.1×10¹³ strands/cm². For the 0.1 uM reaction, the final coverage was 2.2×10¹² strands/cm². In addition to bulk concentration of oligonucleotide, the realized coverages are expected to depend on the extent of generation by DTT of reactive thiol groups from the disulfide termini on the as-received oligonucleotides (see Example 2) as well as any subsequent thiol oxidation that may take place prior to immobilization^{17,51}. Although these other factors were not analyzed in detail, the results demonstrate that a range of surface coverage up to very dense layers is attainable with the developed chemistry.

While inclusion of a thiol endgroup enhanced immobilization, FIG. 8 shows that significant, ˜1.2×10¹² strands/cm², coverage was reached even with the P oligonucleotide, which lacked a thiol. Therefore, the unmodified DNA oligonucleotides are capable of either physically adsorbing or covalently binding to the surface via a site other than a terminal thiol (e.g. a base amine or a 3′ hydroxyl). Chrisey et al. reported that physically adsorbed oligonucleotides on maleimide-derivatized aminosilane surfaces could be desorbed by immersion in high ionic strength solutions⁵². In the present study, oligonucleotides were immobilized from 1 M NaCl buffer, so that physical adsorption was expected to be similarly suppressed, suggesting that direct covalent attachment is the more likely explanation for the observed coverages of the P oligonucleotide. Hybridization of the above oligonucleotide layers to complementary and noncomplementary target strands was performed simultaneously using different regions of the same slide, as defined by separate silicone O-rings. As FIG. 9 shows, hybridization on PSH surfaces was sequence specific with signals from noncomplementary targets less than 10% of those from the fully complementary sequence. The extents of hybridization are interesting. On the high coverage surface with 2.1×10¹³ PSH strands/cm², only 13% of the PSH oligonucleotides underwent hybridization with complementary TC targets. The low yield likely reflects steric crowding as the theoretical jamming coverage for double-stranded DNA is about 3×10¹³ per cm², close to the density of the PSH layer. Other reports have noted decreases in hybridization yields at coverages exceeding ˜5×10¹² strands/cm^{2 53-56}.

In contrast, based on results from oligonucleotide layers assembled on gold films^{5,53}, close to 100% hybridization yields should be achievable at the lower coverage of 2.2×10¹² strands/cm², presuming each immobilized PSH strand is active and accessible. That hybridization yields of only 40% were observed suggests that 60% of the surface-tethered strands were inactive. A possible source of strand deactivation, though certainly not an exclusive one, is depicted in FIG. 10. If, as illustrated, a strand becomes bound at more than one point along its backbone then the end-to-end distance of the segment between the immobilized points is constrained. If this distance deviates significantly from the length required to accommodate a rigid, hybridized duplex then, as discussed by Bünemann⁵⁷, binding of a target TC strand may be prevented. Here, 60% of inactive strands represents a coverage of 1.3×10¹² strands/cm². This value is comparable to the 1.2×10¹² strands/cm² coverage obtained with nonthiolated P oligonucleotides (FIG. 8). In other words, if inactivity toward hybridization is attributed to formation of surface crosslinks via multiple sites (FIG. 10), the surface density of crosslinks required is consistent with that needed to explain immobilization of the P oligonucleotides.

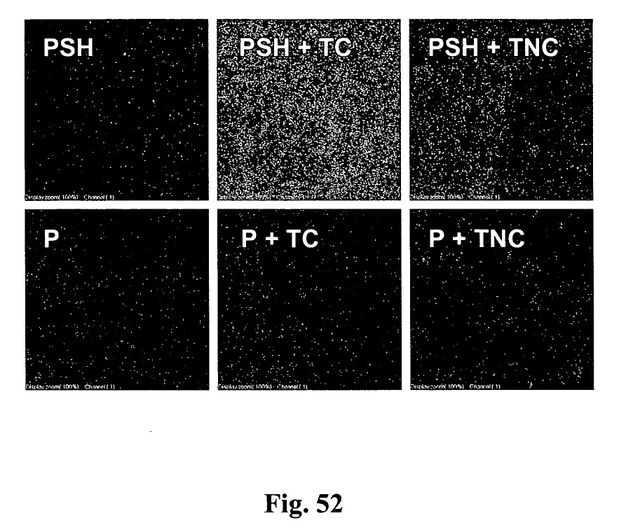

Visual illustration of the hybridization activity of surfaces functionalized with PSH and P oligonucleotides is provided in FIG. 52, which shows fluorescence images captured by confocal microscopy. Prior to hybridization, both probe surfaces appeared dark (FIG. 52 left). After 30 hr exposure to 0.1 μM solutions of fully complementary TC target, followed by washing in PBS buffer, the PSH surface was clearly active and hybridized with TC target (FIG. 52 top middle). In contrast, immobilized P oligonucleotides were inactive (FIG. 52 bottom middle). This lack of hybridization may reflect immobilization of the P oligonucleotides through an internal site in a manner that interferes with their ability to bind target. This is consistent with absence of a thiol endgroup on these strands to direct site-specific attachment via a terminus. Neither oligonucleotide showed significant activity toward hybridization with the noncomplementary TNC target (FIG. 52 right).

III.4. Summary. Precise tailoring of siliceous surfaces with biologically relevant species including proteins, peptides, and nucleic acids underpins applications in separations, diagnostics, sensing, and is important to a fundamental understanding of such systems. The research reported in this Example helps advance capabilities in these areas. In particular, heterobifunctional crosslinker chemistries were compared in their ability to activate surfaces for immobilization of DNA oligonucleotides. amine groups were first introduced to silica or glass surfaces by silylation with aminopropyltriethoxysilane (APTES) or aminopropyldimethylethoxysilane (APDMES). APTES modified surfaces were shown to be preferable for reasons of stability. Several kinds of crosslinkers, incorporating amine- and thiol-reactive sites, were tested for attachment of thiol-terminated oligonucleotides. A heterobifunctional crosslinker possessing an amine-reactive NHS-ester and a thiol-reactive maleimide performed poorly on account of a side reaction between surface APTES amines and its maleimide site, leaving very few active maleimides for subsequent attachment of thiolated oligonucleotides. In contrast, under comparable conditions when isocyanate was used as the amine-reactive linker moiety instead of an NHS-ester, excellent preservation of maleimide activity was observed. This advantage evidently derives from facile reaction of the isocyanate with surface amines, leading to rapid saturation of the reactive amine nucleophiles and thus preventing their side reaction with maleimide groups. Single-stranded oligonucleotides immobilized on thus activated surfaces hybridized complementary strands from solution at yields up to 40%.

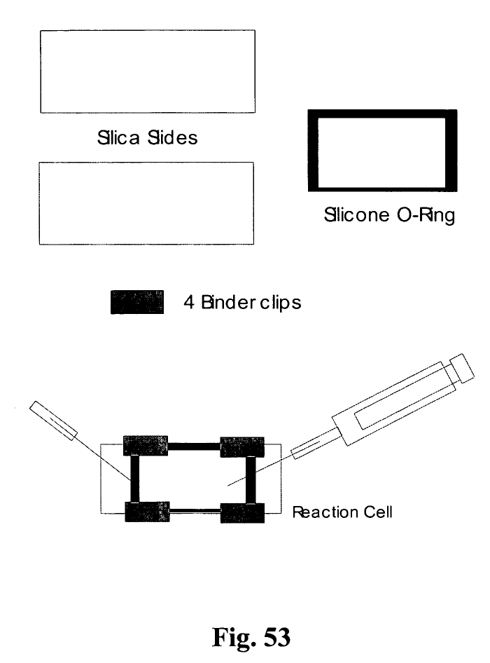

III.5. Appendix: DNA Attachment to Glass Slides. FIG. 53 shows the reaction cell used for modification of glass slides. After cleaning of the slides, the cell was assembled with two slides sandwiching the silicone O-ring. Binder clips were used to hold the assembly together as shown. Syringes, one used for filling and the other for venting of the cell, were inserted through the O-ring and used to introduce APTES solutions, crosslinker solutions, DNA buffers, or washing solvents as needed. Dedicated syringes were used for each type of liquid so as to avoid cross-contamination. Stirring of cell contents was best accomplished by leaving a small air bubble after filling of the cell. The bubble was then used to push the liquid around the cell interior. This required removal of the syringe needles in order to seal the cell and then spinning the cell on a vertical rotary mount.

In order to quantify how much DNA attached to the glass slides, a fluorescence-based kit from Molecular Probes called Oligreen® was used. The propriety mix includes a fluorescent dye that binds to single-stranded DNA, what activates the dye's fluorescence. When determining concentration of DNA via this method, a calibration must be performed that relates fluorescence for a specific oligonucleotide with its concentration.

Calibration is performed by preparing a 1000 ng/ml master oligonucleotide solution, whose concentration is confirmed by absorbance measurement at 260 nm using a known (or calculated) extinction coefficient for the DNA sequence of interest at this wavelength. The fluorescence of this master solution is determined following the manufacturer recommended Oligreen® protocol. Next, the master oligonucleotide solution is progressively diluted, noting the dilution factors so that the concentration remains known. A curve of fluorescence vs. concentration is determined by measuring the Oligreen® signal from each solution of lower concentration. In between Oligreen® measurements, the fluorescence cuvettes are cleaned by washing with buffer and ethanol, and drying thoroughly under compressed nitrogen stream. Dilutions were performed directly in the cuvette in order to minimize potential for contamination and to avoid DNA losses due to adsorption to walls of other containers. For reproducibility, the cuvette was always placed into the fluorometer in the same orientation.

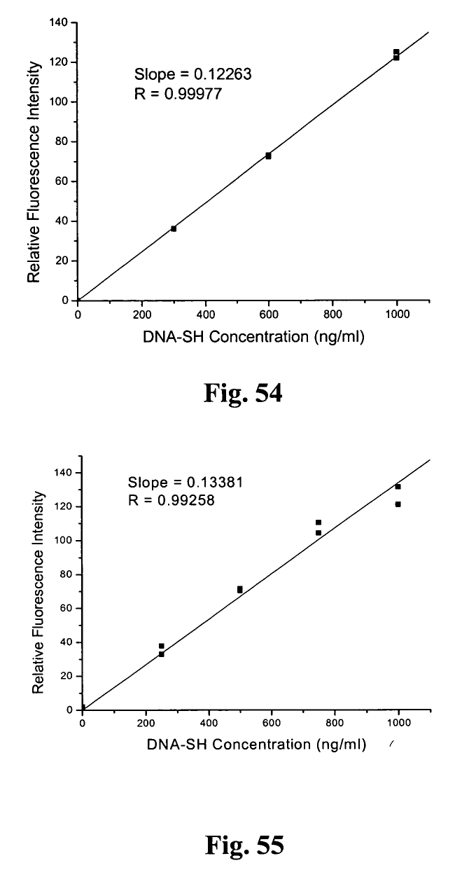

Importantly, it was found that plastic cuvettes posed reproducibility problems compared to quartz cuvettes. Likely, the DNA oligonucleotides adsorbed more strongly to the disposable polystyrene (PS) cuvettes. FIGS. 54 and 55 show Oligreen® calibration curves measured using a disposable polystyrene and a quartz cuvette, respectively. Identical solutions and procedures were employed for both sets of data. At each concentration there are two measurements. The measurements in the PS cuvette exhibit about a 5-10% variation, while there is almost no difference between the measurements in the quartz cuvette. Therefore, quartz cuvettes were employed.

### REFERENCES FOR EXAMPLE 3

(1) Southern, E. M. Trends in Genetics 1996, 12, 110.
(2) Lockhart, D. J.; Winzeler, E. A. Nature 2000, 405, 827.
(3) Staudt, L. M.; Brown, P. O. Annu. Rev. Immunol. 2000, 18, 829.
(4) Steel, A. B.; Levicky, R. L.; Herne, T. M.; Tarlov, M. J. Biophys. J. 2000, 79, 975.
(5) Levicky, R.; Herne, T. M.; Tarlov, M. J.; Satija, S. K. J. Am. Chem. Soc. 1998, 120, 9787.
(6) Georgiadis, R.; Peterlinz, K. P.; Peterson, A. W. J. Am. Chem. Soc. 2000, 122, 3166.
(7) Heaton, R. J.; Peterson, A. W.; Georgiadis, R. M. Proc. Natl. Acad. Sci. U.S.A. 2001, 98, 3701.
(8) Chan, V.; McKenzie, S. E.; Surrey, S.; Fortina, P.; Graves, D. J. J. Colloid Interface Sci. 1998, 203, 197.
(9) Su, H.-J.; Surrey, S.; McKenzie, S. E.; Fortina, P.; Graves, D. J. Electrophoresis 2002, 23, 1551.
(10) Watterson, J. H.; Piunno, P. A. E.; Wust, C. C.; Krull, U. J. Langmuir 2000, 16, 4984.
(11) Piunno, P. A. E.; Watterson, J.; Wust, C. C.; Krull, U. J. Anal. Chim. Acta 1999, 400, 73.
(12) Walker, H. W.; Grant, S. B. Langmuir 1995, 11, 3772.
(13) Beaucage, S. L. Cur. Medicinal Chem. 2001, 8, 1213.
(14) Henke, L.; Krull, U. J. Can. J. Anal. Sci. Spectrosc. 1999, 44, 61.
(15) Tarlov, M. J.; Steel, A. B. In Biomolecular Films: Design, Function, and Applications; Rusling, J. F., Ed.; Marcel Dekker: New York, 2003, pp 545-608.
(16) Yang, M. S.; Kong, R. Y. C.; Kazmi, N.; Leung, A. K. C. Chem. Lett. 1998, 257.
(17) Chrisey, L. A.; Lee, G. U.; O'Ferrall, C. E. Nucl. Acids Res. 1996, 24, 3031.
(18) Adessi, C.; Matton, G.; Ayala, G.; Turcatti, G.; Mermod, J.-J.; Mayer, P.; Kawashima, E. Nucl. Acids Res. 2000, 28, e87.
(19) Okamoto, T.; Suzuki, T.; Yamamoto, N. Nat. Biotechnol. 2000, 18, 438.
(20) Cavic, B. A.; McGovern, M. E.; Nisman, R.; Thompson, M. Analyst 2001, 126, 485.
(21) Andreadis, J. D.; Chrisey, L. A. Nucl. Acids Res. 2000, 28, e5.
(22) Oh, S. J.; Cho, S. J.; Kim, C. O.; Park, J. W. Langmuir 2002, 18, 1764.
(23) Xiao, S.-J.; Textor, M.; Spencer, N. D. Langmuir 1998, 14, 5507.
(24) MacBeath, G.; Koehler, A. N.; Schreiber, S. L. J. Am. Chem. Soc. 1999, 121, 7967.
(25) Hong, H. G.; Bohn, P. W.; Sligar, S. G. Anal. Chem. 1993, 65, 1635.
(26) Vansant, E. F.; Van Der Voort, P.; Vrancken, K. C. Characterization and Chemical Modification of the Silica Surface; Elsevier: New York, 1995; p. 65.
(27) Lin-Vien, D.; Colthup, N. B.; Fateley, W. G.; Grasselli, J. G. The Handbook of Infrared and Raman Characteristic Frequencies of Organic Molecules; Academic Press: New York, 1991; p. 281.
(28) Gorski, D.; Klemm, E.; Fink, P.; Horhold, H.-H. J. Coll. Int. Sci. 1988, 126, 445.
(29) Chiang, C. H.; Ishida, H.; Koenig, J. L. J. Colloid Interface Sci. 1980, 74, 396.
(30) White, L. D.; Tripp, C. P. J. Colloid Interface Sci. 2000, 232, 400.
(31) de Zea Bermudez, V.; Carlos, L. D.; Alcacer, L. Chem. Mater. 1999, 11, 569.
(32) Parker, S. F.; Mason, S. M.; Williams, K. P. J. Spectrochim. Acta 1990, 46A, 315.
(33) Ellman, G. L. Arch. Biochem. Biophys. 1959, 82, 70.
(34) Riddles, P. W.; Blakeley, R. L.; Zerner, B. Anal. Biochem. 1979, 94, 75.
(35) McGall, G. H.; Barone, A. D.; Diggelmann, M.; Fodor, S. P. A.; Gentalen, E.; Ngo, N. J. Am. Chem. Soc. 1997, 119, 5081.
(36) Blitz, J.; Murthy, R. S. S.; Leyden, D. E. J. Am. Chem. Soc. 1987, 109, 7141.
(37) Vansant, E. F.; Van Der Voort, P.; Vrancken, K. C. Characterization and Chemical Modification of the Silica Surface; Elsevier: New York, 1995; p. 243.
(38) Caravajal, G. S.; Leyden, D. E.; Quinting, G. R.; Maciel, G. E. Anal. Chem. 1988, 60, 1776.
(39) Kallury, K. M. R.; Macdonald, P. M.; Thompson, M. Langmuir 1994, 10, 492.
(40) Vandenberg, E. T.; Bertilsson, L.; Liedberg, B.; Uvdal, K.; Erlandsson, R.; Elwing, H.; Lundstrom, I. J. Coll. Interfac. Sci. 1991, 147, 103.
(41) Waddell, T. G.; Leyden, D. E.; DeBello, M. T. J. Am. Chem. Soc. 1981, 103, 5303-5307.)
(42) Moon, J. H.; Kim, J. H.; Kim, K. J.; Kang, T. H.; Kim, B.; Kim, C. H.; Hahn, J. H.; Park, J. W. Langmuir 1997, 13, 4305.
(43) Trens, P.; Denoyel, R. Langmuir 1996, 12, 2781.
(44) Mueller, R.; Kammler, H. K.; Wegner, K.; Pratsinis, S. E. Langmuir 2003, 19, 160.
(45) Zhuravlev, L. T. Colloid Surf. A-Physicochem. Eng. Asp. 2000, 173, 1.
(46) Vrancken, K. C.; Van Der Voort, P.; Possemiers, K.; Vansant, E. F. J. Colloid Interface Sci. 1995, 174, 86.
(47) Annunziato, M. E.; Patel, U.S.; Ranade, M.; Palumbo, P. S. Bioconjugate Chem. 1993, 4, 212.
(48) Smith, M. B.; March, J. March's Advanced Organic Chemistry; 5th ed.; John Wiley & Sons, Inc.: New York, 2001; p. 1178.
(49) Crivello, J. V. J. Polym. Sci. Pol. Chem. 1973, 11, 1185.
(50) Brinkley, M. Bioconjugate Chem. 1992, 3, 2.
(51) O'Donnell, M. J.; Tang, K.; Koster, H.; Smith, C. L.; Cantor, C. R. Anal. Chem. 1997, 69, 2438.
(52) Chrisey, L. A.; Roberts, P. M.; Benezra, V. I.; Dressick, W. J.; Dulcey, C. S.; Calvert, J. M. In Materials Research Society Symposium Proceedings; Alper, M., Bayley, H., Kaplan, D., Navia, M., Eds.; Materials Research Society: Pittsburgh, Pa., 1994; Vol. 330, pp 179.
(53) Walsh, M. K.; Wang, X.; Weimer, B. C. J. Biochem. Biophys. Methods 2001, 47, 221.
(54) Steel, A. B.; Herne, T. M.; Tarlov, M. J. Anal. Chem. 1998, 70, 4670.
(55) Podyminogin, M. A.; Lukhtanov, E. A.; Reed, M. W. Nucl. Acids Res. 2001, 29, 5090.
(56) Beattie, W. G.; Meng, L.; Turner, S. L.; Varma, R. S.; Dao, D. D.; Beattie, K. L. Molec. Biotechnol. 1995, 4, 213.
(57) Bunemann, H. Nucl. Acids Res. 1982, 10, 7181.

### EXAMPLE 4

### Ultrathin Films of Poly(Mercaptosiloxanes) for Robust Immobilization of Biological Polyelectrolytes on Gold

An important aspect in modification of solid surfaces with biological polymers is to tether the molecule of interest permanently and in a well-defined attachment geometry. Gold is perhaps the most popular metal support for research applications yet suffers from a lack of methods for producing robust biomolecular films that are capable of withstanding prolonged use or elevated temperatures. The issue of stability is addressed by first self-assembling a 2 nm thick layer of a thiol-derivatized polysiloxane, poly(mercaptopropyl)methylsiloxane (PMPMS), on the gold support. Multivalent binding of the polymer thiols to the gold, combined with the polymer's hydrophobic nature, cause it to irreversibly adhere to the metal support. Thiol-terminated DNA oligonucleotides are subsequently covalently linked to the PMPMS film using bismaleimide crosslinkers. Immobilization coverages of ˜1×10¹³ strands/cm² have been demonstrated. More notably, the DNA monolayers are capable of withstanding prolonged exposure to near 100° C. conditions with minimal loss of strands from the solid support. Immobilized oligonucleotides retain ability to undergo sequence-selective hybridization, opening up applications of these stable monolayers in diagnostic and related areas.

Derivatization of interfaces with synthetic and biological molecules underpins applications in separations, nanofabrication, sensing, and molecular diagnostics. In one embodiment, the orientation, permanency of attachment, and local environment are precisely specified and reproducibly maintained from one molecule to the next. A number of methods have been developed for assembling nucleic acid monolayers on metal (e.g. gold), polymer, and glass-like (e.g. silica, microscope slides, oxidized silicon) surfaces¹⁻⁴. Siliceous supports are widely available, inexpensive, and well-suited to fluorescence detection. The conductivity of metal supports, on the other hand, facilitates electrochemical detection schemes and provides means to electronically tune the organization and function of nucleic acid films⁵⁻¹¹.

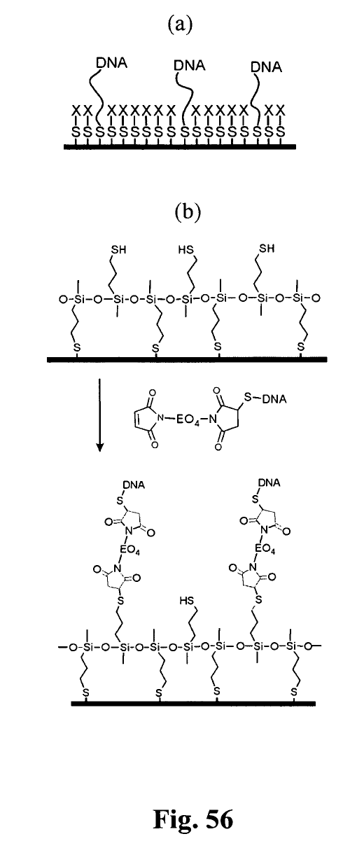

Gold is perhaps the most common metal support employed for immobilization of polynucleic acids, with attachment of DNA strands often mediated via chemisorption of a single thiol (—SH) moiety to the metal¹²⁻⁵¹ (e.g. FIG. 56a). Methods relying on a single thiol-gold linkage to tether the oligonucleotide to the surface, however, are faced with significant limitations in terms of permanence and hence suitability in applications. Mirkin and colleagues investigated dispersions of gold particles functionalized with a shell of DNA oligomers in aqueous buffers, and found enhanced stability to particle aggregation when multiple sulfur-gold bonds were used to attach each DNA strand^{52,53}. These results demonstrate that improved stability of bound strands is achieved by employing a multivalent attachment scheme, as opposed to immobilization via a single sulfur atom or thiol moiety. Other investigators have similarly noted the significant lability of thiol-tethered DNA monolayers on gold⁵⁴. Sulfur atoms are susceptible to oxidation^{55,56}, e.g. due to ambient ozone⁵⁷⁻⁵⁹, which may lead to breakage of the surface linkage and subsequent removal of the thiolated compound from the surface.

This Example provides a preparation of stable DNA monolayers on gold. This Example demonstrates robustness of these monolayers at 90° C. conditions, a requirement for applications such as polymerase chain reaction (PCR) that require elevated temperatures. Site-specificity of strand immobilization via one terminus is confirmed, and activity of surface-tethered single-stranded oligonucleotides toward hybridization with strands in a contacting buffer is demonstrated. Moreover, the surfaces exhibit low nonspecific adsorption of DNA. An overview of the DNA anchoring strategy is depicted in FIG. 56b. The first step involves self-assembly of a base layer of poly(mercaptopropyl)methylsiloxane (PMPMS) on the gold surface. The hydrophobic PMPMS binds to the metal through multiple thiol-gold linkages. In a second step, maleimide-terminated oligonucleotides react with remnant PMPMS thiols to form stable thioether bonds. This simple approach replaces one sulfur-gold bond, typically the weak “link” of the conventional strategy (FIG. 56a), with a highly multivalent attachment. A metal surface modified with PMPMS is also shown to lend itself to immobilization of thiolated oligonucleotides via formation of disulfide bonds. XPS is used as a primary characterization tool at all stages of surface modification.

Compared to prior reports in which oligonucleotides capable of divalent⁵² or trivalent⁵³ chemisorption to metal supports were used to enhance stability, the present strategy is synthetically simpler as it significantly decreases labor associated with oligonucleotide modification. Multivalent sulfur-gold interactions were also employed by Leavitt et al. to robustly immobilize kilobase DNA molecules on gold surfaces, using sulfur-modified phosphate groups in the DNA backbone⁶⁰. These investigators tethered the DNA through a large number of backbone sites to optimize conditions for DNA imaging via scanning probe microscopy. In contrast, the method in this Example seeks to minimize conformational constraints by site-specific attachment via one strand terminus. An end-attachment geometry is anticipated to be favorable for hybridization assays and related applications requiring interaction between the bound DNA and species present in bulk buffer. Previously, thiolated poly(L-lysine) was employed by Wink et al to modify gold supports for subsequent immobilization of DNA by electrostatic interactions⁶¹. Here, a thiolated polymer (PMPMS) is employed in a covalent attachment scheme.

Experimental Methods.

Materials. Poly(mercaptopropyl)methylsiloxane (PMPMS; degree of polymerization ˜40) was from Gelest Inc. Dithiothreitol (DTT) and the bismaleimide crosslinker bis-maleimidotetraethyleneglycol (BM(PEO)₄) were from Pierce Biotechnology. Oligonucleotides were provided by Qiagen Operon and included purification by HPLC.

Thiol-terminated oligonucleotides were prepared from commercial precursors with a 3′ disulfide terminus. The 20mer oligonucleotide 5′CGT TGT AAA ACG ACG GCC AG-(CH₂)₃—SS—(CH₂)₃OH 3′ (SEQ ID NO. 1), at a 20 μM concentration in 1× saline sodium citrate buffer containing 1M NaCl (SSC1M; 0.015 M sodium citrate, 1M NaCl, pH 7.0), was treated with a 1000-fold excess of DTT for 1 hr to reduce the disulfide and liberate the terminal thiol. Excess DTT was removed by size exclusion separation on a PD-10 column (Amersham Pharmacia) using SSC1M buffer. 2.5 ml of eluent containing thiol-terminated DNA (DNA-SH) was collected at a final concentration of about 10 μM and used immediately.

Maleimide-terminated oligonucleotides were prepared by adding to the DNA-SH eluent a solution of BM(PEO)₄ in SSC1M to obtain a 100-fold excess of the linker over oligonucleotide thiols. After a 2 hr reaction time, DNA-S-BM(PEO)4 conjugates were recovered by a second pass through a PD-10 column and used immediately for surface attachment. The final concentration of DNA-S-BM(PEO)4 was about 5 mM. In addition to oligonucleotides with a maleimide at the 3′ terminus, the recovered eluent contained a small fraction of oligonucleotide dimers where two strands crosslinked via a single BM(PEO)4 molecule, yielding DNA-S-BM(PEO)4-S-DNA. The dimers do not bear free maleimide or thiol groups and thus were unreactive toward PMPMS thiols.

Surface Modification. Metal films, comprising a 10 nm Cr adhesion sublayer and a 250 nm thick Au top-layer, were thermally evaporated on glass slides and annealed at 250° C. for 2 hours before cleaning in hot piranha solution for 15 minutes (7:2:1 H₂SO₄:H₂O:H₂O₂). CAUTION: Piranha solution is highly corrosive and should not be stored in tightly sealed containers on account of gas evolution. Piranha-cleaned surfaces were extensively rinsed with deonized water from a Millipore Biocell system and immersed, without drying, for 20 minutes in ethanol to reduce any gold oxide formed⁶². Finally, gold films were transferred, again without drying, into PMPMS solutions in toluene for the first step of surface modification.

Freshly cleaned gold surfaces were immersed in 10 mM solutions (by monomer) of PMPMS in toluene for 1 hour, rinsed extensively with toluene, and dried under a compressed nitrogen stream. PMPMS surfaces were exposed to solutions (recovered as eluent from PD-10 columns) of DNA-S-BM(PEO)₄ or DNA-SH oligonucleotides overnight, at a typical concentration of 5 μM oligonucleotide in SSC1M buffer. After immobilization of the DNA, the surfaces were thoroughly rinsed with buffer followed by deionized water, and dried with a nitrogen stream. PMPMS monolayers reacted with just the BM(PEO)₄ crosslinker were prepared similarly by exposing PMPMS-modified surfaces to 1 mg/ml crosslinker in SSC1M, using a 2 hr reaction time.

Surface Characterization. X-ray photoelectron spectroscopy measurements were performed on a PHI 5500×PS instrument equipped with an Al X-ray monochromatic source (Al Kα line, 1486.6 eV) and a spherical capacitor energy analyzer (SCA) at 58.70 eV pass energy and 0.25 eV/step resolution. High resolution multicomponent scans for gold (Au 4d and 4f), carbon (C 1s), silicon (Si 2p), oxygen (O 1s), sulfur (S 2p), phosphorus (P 2p), and nitrogen (N 1s) were obtained at 45° unless indicated otherwise. Integration times were 5 minutes for C, Au, and O; 15 minutes for Si, S, and N; 60 minutes for P. Binding energies were referenced to Au 4f set at 83.9 eV. Elemental detection limits were about 0.1% of total XPS signal. Oxygen signals typically exceeded, in a variable manner, stoichiometrically predicted ratios. This observation was attributed to oxidation of PMPMS sulfur atoms⁵⁷⁻⁵⁹. For this reason, the oxygen signal was not used in quantitative analysis. XPS traces were deconvoluted into separate peaks using XPS Peak software. A convolution of Gaussian and Lorentzian peak shapes was employed, with the signal baseline modeled using Shirley and linear functions. Analysis of our XPS results closely parallels the methods of Petrovykh et al.⁶³, who correlated XPS and infrared spectroscopy data from DNA layers chemisorbed directly to gold via a terminal thiol moiety.

Electrochemical characterization of modified gold supports was carried out with a Parstat 2263 potentiostat/galvanostat/frequency response analyzer (Princeton Applied Research) in a three electrode configuration. A silver wire coated with AgCl served as a pseudoreference electrode, with a Pt wire counterelectrode. The reference and counter electrodes were inserted through a one-piece silicone gasket seal into a fully enclosed circular chamber of 0.54 cm² area and 0.3 cm height filled with electrolyte; the “floor” of the chamber was formed by the working surface while the other side was sealed with a clean glass slide. Cyclic voltamograms (CVs) were measured at 50 mV/sec in 100 mM potassium phosphate buffer at pH 10. Impedance measurements were performed from 200 kHz to 1 Hz with an AC amplitude of 5 mV and at a DC bias of 150 mV versus the Ag/AgCl wire.

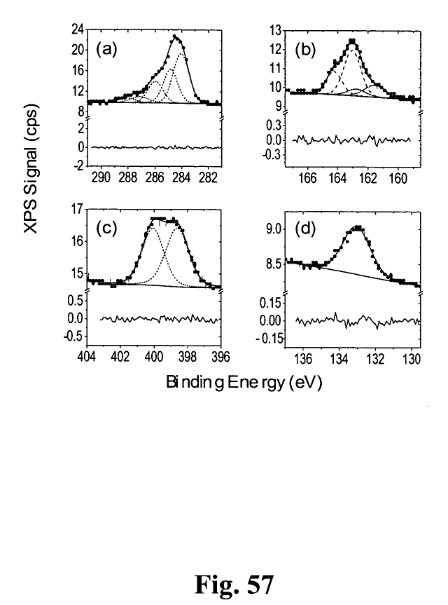

Results and Discussion. XPS served as a primary characterization tool for monitoring the composition of modified surfaces, both after attachment of PMPMS and DNA. FIG. 57 depicts typical high resolution XPS traces for C 1s, S 2p, N 1s, and P 2p signals measured on a gold surface derivatized with PMPMS followed by reaction with DNA-S-BM(PEO)₄. Satisfactory fit of C 1s data (FIG. 57a) for this film required five components at binding energies of 284.1 eV (44%), 284.8 eV (28%), 286.0 eV (18%), 287.1 eV (6%), and 288.1 eV (4%). C 1s signal from pure PMPMS films peaked at 284.4 eV, in agreement with prior studies.⁶⁴ Accordingly, the low binding energy components at 284.1 eV and 284.8 eV are attributed to PMPMS and to DNA where the carbon is not involved in bonds with oxygen or nitrogen. Allowing for a systematic shift of +0.5 eV relative to our data, the remaining three components at higher binding energies were also identified by Cavic et al in XPS spectra of DNA monolayers on silicon supports⁶⁵. These components must originate from carbon atoms involved in various bonding arrangements with oxygen and nitrogen (C—O, N—C—O, N═C—N,N—C═O, etc.), though we have not attempted to correlate specific spectral features with carbon chemical environments found in DNA.

Two components, present at comparable amounts and located at 398.6 eV and 400.1 eV, were required to fit the N 1s signal (FIG. 57c). Similar results were obtained for DNA-SH immobilized via disulfide formation to free PMPMS thiols. Cavic et al also found two components in DNA N 1s spectra, and assigned them to intracyclic and exocyclic nitrogen atoms⁶⁵. This assignment, however, appears too simplistic for our data as it predicts a peak:peak ratio of 1:3.8 for our probe sequence, contradicting the approximately 1:1 ratio observed experimentally (FIG. 57c). Petrovykh et al carried out an investigation of the N 1s spectra of dT₂₅ homo-oligonucleotides on gold which, aside from surface-induced effects, could be analyzed in terms of a single principal peak as both N atoms of thymine exhibit similar binding energies⁶³. Clearly, oligonucleotide XPS spectra are sequence-dependent. Indeed, the nitrogen atoms in DNA produce binding energies between ˜398 eV and ˜402 eV, depending on the nitrogen bonding configuration⁶⁶. Finally, decomposition of the S 2p traces is relevant to understanding organization of the PMPMS layer and will be discussed below.

TABLE 4.1
Relative XPS Elemental Signals for Modified Gold Surfaces^{a}

| Sample | P/Si | N/Si | C/Si |
|---|---|---|---|
| PMPMS | ND^{b} | ND^{b} | 4.34 |
| PMPMS + BM(PEO)₄ | ND^{b} | 0.61 | 8.66 |
| PMPMS + DNA-S-BM(PEO)₄ | 0.18 | 0.79 | 7.00 |
| PMPMS + DNA control | ND^{b} | ND^{b} | 4.77 |

^{a}measured at 45°
^{b}ND: not detectable

Table 4.1 summarizes integrated P 2p, N 1s, and C 1s signals for (i) a pure PMPMS layer, (ii) a PMPMS layer reacted with just BM(PEO)₄ linker, (iii) a PMPMS layer reacted with DNA-S-BM(PEO)₄ conjugates, and (iv) a control experiment testing site-specificity of oligonucleotide attachment (see below). The 3rd row of Table 4.1 corresponds to the schematic layer structure depicted in FIG. 56b bottom. All signals have been normalized to that of Si 2p.

PMPMS Films. Pure PMPMS films (top row Table 4.1) had no detectable emission from P or N, consistent with PMPMS lacking these elements. The stoichiometric C/Si ratio for PMPMS is 4.0, compared to somewhat higher values measured experimentally (Table 4.1). This deviation likely reflects adventitious contamination introduced during handling of the samples in ambient environment prior to insertion into the XPS apparatus. The PMPMS S 2p signal (FIG. 57b) could be decomposed into at most three separate components identified with sulfur atoms that are (i) bound to Au (thiolate S), (ii) present as thiols or disulfides, and (iii) oxidized. The three S 2p components were each modeled as a peak doublet (corresponding to S 2p3/2 and S 2p_{1/2}) with one peak ½ the size of the other in area and shifted by 1.2 eV⁶⁷. The oxidized signal varied from sample to sample. In the data of FIG. 57b there is virtually no oxidized sulfur (S 2p3/2 position ˜168 eV), but bound thiolate (S 2p_{3/2} position 161.6 eV) and thiol/disulfide sulfurs (163.1 eV) are both evident. The binding energies observed in the present study are consistent with prior XPS results on similar PMPMS films⁶⁷.

The bound thiolate signal was typically ˜20% of the total S 2p intensity, implying that one in five monomers was bound to the metal support. However, this estimate represents a lower limit because the measured thiolate signal is weakened by transmission through the PMPMS overlayer. Regardless, the S 2p decomposition provides strong evidence that each PMPMS chain is multivalently adsorbed to the gold layer, with more than eight bonds per 40-monomer long chain on average.

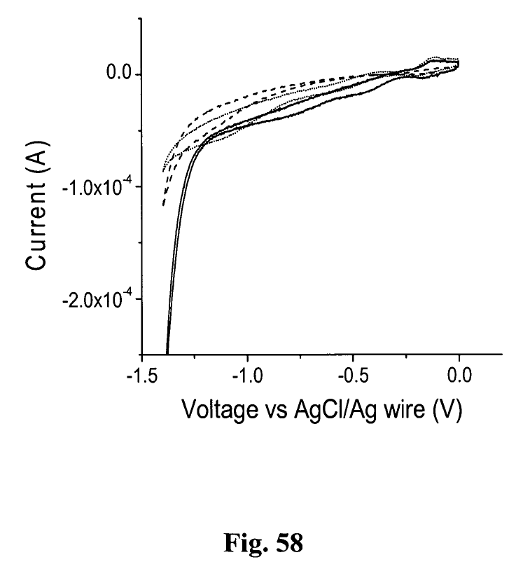

PMPMS films were further characterized with electrochemical methods to provide a qualitative measure of their structural consolidation. Measurements were performed in 100 mM phosphate buffer at a pH of 10. For comparison, cyclic voltametry (CV) was also carried out on bare gold surfaces and on gold surfaces bearing a self-assembled monolayer (SAM) of mercaptohexanol (MCH), formed by a 1 hour immersion in 1 mM MCH solution in deionized water. As shown in FIG. 58, the PMPMS film blocks faradaic processes compared to a bare Au electrode, though to a lesser extent than MCH. Qualitatively, the greater permeability of PMPMS presumably reflects a looser, more disordered packing of these polymeric chains compared to the smaller MCH molecules.

Similar conclusions were reached from impedance measurements of the double layer capacitance C_{dl}. Impedance measurements were performed under nonfaradaic conditions. The data was analyzed in the standard fashion with the interface modeled as a resistor and capacitor in series⁶⁸. The resistor represents barrier to current transfer through the bulk electrolyte, while the capacitance represents charging of the solid-liquid interface. Under these conditions, C_{dl} is asymptotically estimated from the magnitude of the impedance Z at driving frequency ω=1 rad/sec via |Z|=1/C_{dl}. The measured C_{dl} values were 4.6 μF/cm² for the MCH SAM, 21 μF/cm² for the PMPMS layer, and 110 μF/cm² for bare Au. Conceptualizing the organic (PMPMS or MCH) coating as a thin dielectric film, the capacitance values imply a smaller effective dielectric thickness for a layer of PMPMS. As the PMPMS physical thickness of 2.4 nm (estimated from angle-resolved XPS; see below) is about twice that for MCH²⁰, the impedance measurements likewise indicate significantly greater permeability of the PMPMS compared to the SAM.

PMPMS Films Modified with BM(PEO)₄. XPS spectra of PMPMS films reacted with BM(PEO)₄ exhibited the expected N 1s emission at 400.5 eV, as well as an increase in the C/Si ratio (second row Table 4.1). Subsequent exposure of the BM(PEO)₄ modified PMPMS to thiol-terminated DNA-SH oligonucleotides failed to produce a DNA P 2p signal, indicating that little if any DNA attached. Considering sensitivity limitations of the XPS instrument, this result implies that the DNA coverage was below 1×10¹² molecules/cm². Such a low coverage indicates that DNA thiols were unsuccessful in locating active maleimide groups on the surface to react with; in other words, that a large fraction of the BM(PEO)₄ linkers attached to the PMPMS through both maleimide termini, causing their deactivation. The molecular picture that emerges is one in which the PMPMS film became decorated with short tetraethyleneglycol (PEO₄) loops of the BM(PEO)₄, with the loops tethered at both ends via thioether bonds to the PMPMS polymer.

Nevertheless, even in absence of active maleimides, DNA-SH could still react directly with available PMPMS thiols by forming disulfide (—S—S—) bridges^{29,69,70}. The lack of observable DNA-SH immobilization, therefore, further indicates that few accessible thiols remained on the BM(PEO)₄-reacted PMPMS surface, thus preventing disulfide-mediated attachment of oligonucleotide. In contrast, control experiments in which DNA-SH was exposed to PMPMS surfaces not treated with BM(PEO)₄ produced clear N 1s and P 2p signals (N/Si=0.63; P/Si=0.19). That these strands attached by disulfide linkages was confirmed by control experiments in which oligonucleotides with an identical sequence, but lacking a terminal thiol, failed to attach (N, P signals below detection).

To summarize, under the investigated conditions reaction of PMPMS films with BM(PEO)₄ results in attachment of linker molecules via both ends, at a coverage that effectively consumes all accessible PMPMS thiols. Therefore, pretreatment of a PMPMS surface with BM(PEO)₄, followed by attachment of DNA-SH oligonucleotide, is not an efficient route to assembly of a DNA monolayer whether via thioether (DNA thiol to surface maleimide) or disulfide (DNA thiol to PMPMS thiol) linkages. While DNA-SH is immobilized directly to PMPMS via disulfide formation, such an attachment is susceptible to cleavage by other thiol groups present in solution or at elevated temperatures⁶⁹. For these reasons, a highly-stable immobilization scheme was developed.

PMPMS Films Modified with DNA-S-BM(PEO)₄. Reaction between maleimide-terminated DNA-S-BM(PEO)₄ and PMPMS films produced efficient attachment as evidenced by strong nitrogen and phosphorus signals in XPS spectra (third row Table 4.1). The P to N ratio (1 to 4.4 in Table 4.1) exceeded the value based on stoichiometry, which predicts a ratio of 1 to 3.6. While uncertainties, especially in the weaker P signal with an estimated error of about 10%, partially account for the discrepancy, the excess N 1s intensity likely also reflects co-immobilization of other amine-containing species to the PMPMS. In particular, it is expected that some BM(PEO)₄ remained in the reaction mixture as a result of incomplete separation of linker during clean up (performed on size exclusion columns) after its reaction in 100-fold excess to prepare DNA-S-BM(PEO)₄. Such residual linker would be free to react with the PMPMS layer, elevating the N 1s signals. This explanation is consistent with the fact that experimental and calculated P:N stoichiometries were within experimental error when DNA was immobilized via disulfide formation (exp.: 1 to 3.3; calc.: 1 to 3.6), which did not employ this linker. The discrepancy between observed and expected ratios could be fully explained if 9 linkers attached for each strand of immobilized DNA. The presence of remnant BM(PEO)₄ did not interfere with achieving high DNA coverages (see below) and, indeed, may have had a beneficial side effect as the bismaleimide molecules may crosslink the PMPMS chains to further stabilize the film.

In order to estimate surface coverage of DNA, angle resolved XPS measurements were performed on the same sample at 15°, 18°, 22°, 28°, 35°, 45°, and 80°. The thickness change resulting from DNA attachment was determined by an overlayer/substrate model (equation 4 in reference⁷¹). A constant mean free path of 4.2 nm for the emitted photoelectrons was assumed, measured previously for hydrocarbon alkanethiol monolayers⁷². The calculated thickness values were 2.4 nm for the PMPMS layer prior to DNA attachment and 4.0 nm after reaction with oligonucleotide, an increase of 1.6 nm. However, allowing for attachment of 9 linker molecules per each immobilized strand as estimated above, only 0.9 nm should be attributed to DNA. Using a density of 1.72 g/cm³ for single-stranded DNA⁷³ and molecular weight of 6607 g/gmol, the corresponding estimate of DNA coverage is 1.4×10¹³ strands/cm². This high coverage, realized after immobilization of DNA overnight, exceeds by about a factor of three those typically reported as optimal for oligonucleotide hybridization⁷⁴⁻⁷⁷. Lower coverage is realized by employing shorter reaction times. The 2.4 nm PMPMS thickness is within 30% of values reported for similarly self-assembled polymercaptosiloxane films^{64,78}.

Geometry of attachment, for example via one end as opposed to via an internal site on a strand, influences the ability of an immobilized oligonucleotide to hybridize. In the described protocol, oligonucleotides with maleimide endgroups were generated by reaction of thiol-terminated strands with the bismaleimide BM(PEO)₄. Conceivably, some oligonucleotides may have reacted with BM(PEO)₄ via a site (e.g. a base amine) other than the 3′ terminal thiol, leading to incorporation of BM(PEO)₄ at an internal position. In turn, these strands would attach to PMPMS internally, rather than via the 3′ terminus. This possibility was probed by first reacting oligonucleotides of the same base sequence but without the 3′ thiol modification with BM(PEO)₄, followed by purification of the conjugates and reaction with PMPMS films. Conditions identical to those employed for immobilization of thiol-terminated oligonucleotides were used. The lack of N and P marker signals for DNA in XPS (fourth row Table 4.1) indicates that attachment, if present, was below the detection level of ˜1×10¹² strands/cm². These results indicate that at least 90% of strands are expected to attach regiospecifically via their 3′ termini.

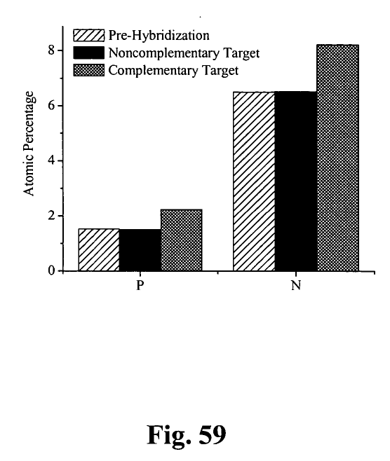

Separate experiments were performed to demonstrate hybridization activity of immobilized strands. Individual pieces taken from the same PMPMS layer reacted with DNA-S-BM(PEO)₄ were exposed to a 7.4×10⁻⁷ M solution of complementary (5′ CTG GCC GTC GTT TTA CAA CG 3′) (SEQ ID NO. 2) or 4.6×10⁻⁷ M solution of noncomplementary (5′ CTA ACT GTT ACC TCG GTC GG 3′) (SEQ ID NO. 3) target strands in a sodium citrate buffer (0.015 M sodium citrate, 1M NaCl, pH 7.0) overnight. The hybridized surfaces were washed thoroughly with buffer solution, followed by a rinse with 4° C. deionized water to remove buffer and salt residue while minimizing dehybridization. Following hybridization with the complementary target, P and N signals increased by 46% and 26%, respectively (FIG. 59). As the N signal also contains contributions from BM(PEO)₄ linkers, the P signal (attributed solely to DNA) is the better gauge of hybridization extent. Hybridization to noncomplementary targets yielded no discernible change in the P and N levels. These results demonstrate ability of PMPMS-tethered DNA to engage in sequence-selective hybridization of target strands.

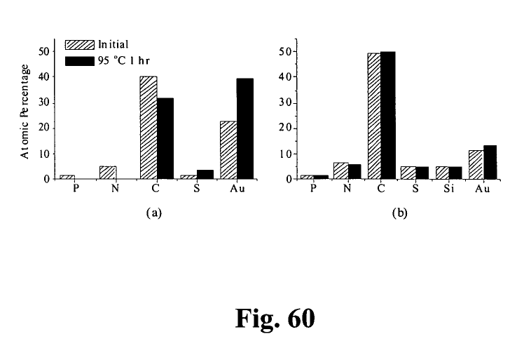

A principal motivation for this work was development of robust DNA monolayers. XPS results from thermal stability studies are presented in FIG. 60. FIG. 60a compares signals before and after 1 hour immersion in 95° C. buffer (0.015 M sodium citrate, 1M NaCl, pH 7.0) for a DNA monolayer assembled via the conventional route (FIG. 56a) using MCH as the surface passivant¹⁷. FIG. 60b depicts analogous results for DNA oligonucleotides assembled on PMPMS-modified gold as in FIG. 56b. Complete loss of P and N signals is observed for the conventionally-prepared DNA monolayer, whereas the PMPMS anchored film exhibits a relatively modest decrease of ˜10%. Post-treatment increase in the Au signal reflects a decrease in the thickness of the organic overlayer. The results of FIG. 60 demonstrate a remarkable enhancement in stability of the current method over immobilization via a single thiol-gold linkage. Moreover, PMPMS-tethered DNA monolayers were stable for at least 7 days (the longest time investigated) when stored under buffer or in air.

In summary, poly(mercaptopropyl)methylsiloxane films on gold were developed to provide highly stable, thin, thiol-rich anchor layers suitable for subsequent attachment of biomolecules. Attachment of DNA oligonucleotides via thiol-maleimide coupling was demonstrated. The developed protocol relies on site-specific immobilization of the DNA strands via one end, and leads to excellent retention of activity toward binding of complementary strands. We have recently generalized this chemistry to attachment of double-stranded molecules carrying a full gene (2000 base pairs). The stability of our method to high temperature conditions should benefit applications (e.g. surface-templated polymerase chain reactions, DNA denaturation) as well as fundamental investigations of biological macromolecules at interfaces.

### REFERENCES OF EXAMPLE 4

(1) Beaucage, S. L. Cur. Medicinal Chem. 2001, 8, 1213.
(2) Tarlov, M. J.; Steel, A. B. In Biomolecular Films: Design, Function, and Applications; Rusling, J. F., Ed.; Marcel Dekker: New York, 2003, pp 545.
(3) Henke, L.; Krull, U. J. Can. J. Anal. Sci. Spectrosc. 1999, 44, 61.
(4) Pirrung, M. C. Angew. Chem.-Int. Edit. 2002, 41, 1276.
(5) Gurtner, C.; Tu, E.; Jamshidi, N.; Haigis, R. W.; Onofrey, T. J.; Edman, C. F.; Sosnowski, R.; Wallace, B.; Heller, M. J. Electrophoresis 2002, 23, 1543.
(6) Sosnowski, R. G.; Tu, E.; Butler, W. F.; O'Connell, J. P.; Heller, M. J. Proc. Natl. Acad. Sci. USA 1997, 94, 1119.
(7) Kelley, S. O.; Barton, J. K.; Jackson, N. M.; McPherson, L. D.; Potter, A. B.; Spain, E. M.; Allen, M. J.; Hill, M. G. Langmuir 1998, 14, 6781.
(8) Heaton, R. J.; Peterson, A. W.; Georgiadis, R. M. Proc. Natl. Acad. Sci. U.S.A. 2001, 98, 3701.
(9) Zhang, Z.-L.; Pang, D.-W.; Zhang, R.-Y. Bioconjugate Chem. 2002, 13, 104.
(10) Wang, J.; Rivas, G.; Jiang, M.; Zhang, X. Langmuir 1999, 15, 6541.
(11) Su, H.-J.; Surrey, S.; McKenzie, S. E.; Fortina, P.; Graves, D. J. Electrophoresis 2002, 23, 1551.
(12) Okahata, Y.; Matsunobu, Y.; Ijiro, K.; Mukae, M.; Murakami, A.; Makino, K. J. Am. Chem. Soc. 1992, 114, 8299.
(13) Hegner, M.; Wagner, P.; Semenza, G. FEBS Lett. 1993, 336, 452.
(14) Hashimoto, K.; Ito, K.; Ishimori, Y. Anal. Chem. 1994, 66, 3830.
(15) Piscevic, D.; Lawall, R.; Veith, M.; Liley, M.; Okahata, Y.; Knoll, W. Appl. Surf Sci. 1995, 90, 425.
(16) Alivisatos, A. P.; Johnsson, K. P.; Peng, X.; Wilson, T. E.; Loweth, C. J.; Bruchez, M. P.; Schultz, P. G. 1996, 382, 609.
(17) Herne, T. M.; Tarlov, M. J. J. Am. Chem. Soc. 1997, 119, 8916.
(18) Elghanian, R.; Storhoff, J. J.; Mucic, R. C.; Letsinger, R. L.; Mirkin, C. A. Science 1997, 277, 1078.
(19) Bamdad, C. Biophys. J. 1998, 75, 1997.
(20) Levicky, R.; Herne, T. M.; Tarlov, M. J.; Satija, S. K. J. Am. Chem. Soc. 1998, 120, 9787.
(21) Yang, M. S.; Yau, H. C. M.; Chan, H. L. Langmuir 1998, 14, 6121.
(22) Kelley, S. O.; Boon, E. M.; Barton, J. K.; Jackson, N. M.; Hill, M. G. Nucleic Acids Res. 1999, 27, 4830.
(23) O'Brien, J. C.; Stickney, J. T.; Porter, M. D. Langmuir 2000, 16, 9559.
(24) Georgiadis, R.; Peterlinz, K. P.; Peterson, A. W. J. Am. Chem. Soc. 2000, 122, 3166.
(25) Peterson, A. W.; Wolf, L. K.; Georgiadis, R. M. J. Am. Chem. Soc. 2002, 124, 14601.
(26) Huang, E.; Satjapipat, M.; Han, S.; Zhou, F. Langmuir 2001, 17, 1215.
(27) Mbindyo, J. K. N.; Reiss, B. D.; Martin, B. R.; Keating, C. D.; Natan, M. J.; Mallouk, T. E. Adv. Mater. 2001, 13, 249.
(28) He, L.; Musick, M. D.; Nicewarner, S. R.; Salinas, F. G.; Benkovic, S. J.; Natan, M. J.; Keating, C. D. J. Am. Chem. Soc. 2000, 122, 9071.
(29) Smith, E. A.; Wanat, M. J.; Cheng, Y.; Barreira, V. P.; Frutos, A. G.; Corn, R. M. Langmuir 2001, 17, 2502.
(30) Thiel, A. J.; Frutos, A. G.; Jordan, C. E.; Corn, R. M.; Smith, L. M. Anal. Chem. 1997, 69, 4948.
(31) Walti, C.; Wirtz, R.; Germishuizen, W. A.; Bailey, D. M. D.; Pepper, M.; Middelberg, A. P. J.; Davies, A. G. Langmuir 2003, 19, 981.
(32) Erts, D.; Polyakov, B.; Olin, H.; Tuite, E. J. Phys. Chem. B 2003, 107, 3591.
(33) Li, C. Z.; Long, Y. T.; Kraatz, H. B.; Lee, J. S. J. Phys. Chem. B 2003, 107, 2291.
(34) Mourougou-Candoni, N.; Naud, C.; Thibaudau, F. Langmuir 2003, 19, 682.
(35) Zhou, D. J.; Sinniah, K.; Abell, C.; Rayment, T. Langmuir 2002, 18, 8278.
(36) Maxwell, D. J.; Taylor, J. R.; Nie, S. M. J. Am. Chem. Soc. 2002, 124, 9606.
(37) Patolsky, F.; Lichtenstein, A.; Willner, I. J. Am. Chem. Soc. 2000, 122, 418.
(38) Wang, L. L.; Silin, V.; Gaigalas, A. K.; Xia, J. L.; Gebeyehu, G. J. Colloid Interface Sci. 2002, 248, 404.
(39) Cho, Y. K.; Kim, S.; Lim, G.; Granick, S. Langmuir 2001, 17, 7732.
(40) Satjapipat, M.; Sanedrin, R.; Zhou, F. M. Langmuir 2001, 17, 7637.
(41) Liu, M. Z.; Amro, N. A.; Chow, C. S.; Liu, G. Y. Nano Lett. 2002, 2, 863.
(42) Cai, H.; Xu, C.; He, P. G.; Fang, Y. Z. J. Electroanal. Chem. 2001, 510, 78.
(43) Csaki, A.; Moller, R.; Straube, W.; Kohler, J. M.; Fritzsche, W. Nucleic Acids Res. 2001, 29, art. no.
(44) Hianik, T.; Gajdos, V.; Krivanek, R.; Oretskaya, T.; Metelev, V.; Volkov, E.; Vadgama, P. Bioelectrochemistry 2001, 53, 199.
(45) Niemeyer, C. M.; Ceyhan, B.; Gao, S.; Chi, L.; Peschel, S.; Simon, U. Colloid Polym. Sci. 2001, 279, 68.
(46) Kertesz, V.; Whittemore, N. A.; Inamati, G. B.; Manoharan, M.; Cook, P. D.; Baker, D. C.; Chambers, J. Q. Electroanalysis 2000, 12, 889.
(47) Fritz, J.; Baller, M. K.; Lang, H. P.; Rothuizen, H.; Vettiger, P.; Meyer, E.; Guntherodt, H. J.; Gerber, C.; Gimzewski, J. K. Science 2000, 288, 316.
(48) Takenaka, S.; Yamashita, K.; Takagi, M.; Uto, Y.; Kondo, H. Anal. Chem. 2000, 72, 1334.
(49) Wang, J.; Rivas, G.; Jiang, M. A.; Zhang, X. J. Langmuir 1999, 15, 6541.
(50) Sun, X. Y.; He, P. G.; Liu, S. H.; Ye, J. N.; Fang, Y. Z. Talanta 1998, 47, 487.
(51) Rekesh, D.; Lyubchenko, Y.; Shlyakhtenko, L. S.; Lindsay, S. M. Biophys. J. 1996, 71, 1079.
(52) Letsinger, R. L.; Elghanian, R.; Viswanadham, G.; Mirkin, C. A. Bioconjug. Chem. 2000, 11, 289.
(53) Li, Z.; Jin, R.; Mirkin, C. A.; Letsinger, R. L. Nucl. Acids Res. 2002, 30, 1558.
(54) Yang, W. S.; Auciello, O.; Butler, J. E.; Cai, W.; Carlisle, J. A.; Gerbi, J.; Gruen, D. M.; Knickerbocker, T.; Lasseter, T. L.; Russell, J. N.; Smith, L. M.; Hamers, R. J. Nat. Mater. 2002, 1, 253.
(55) Li, Y.; Huang, J.; McIver, R. T.; Hemminger, J. C. J. Am. Chem. Soc. 1992, 114, 2428.
(56) Tarlov, M. J.; Newman, J. G. Langmuir 1992, 8, 1398.
(57) Schoenfisch, M. H.; Pemberton, J. E. J. Am. Chem. Soc. 1998, 120, 4502.
(58) Lee, M.-T.; Hsuch, C.-C.; Freund, M. S.; Ferguson, G. S. Langmuir 1998, 14, 6419.
(59) Poirier, G. E.; Herne, T. M.; Miller, C. C.; Tarlov, M. J. J. Am. Chem. Soc. 1999, 121, 9703.
(60) Leavitt, A. J.; Wenzler, L. A.; Williams, J. M.; Beebe, T. P. J. Phys. Chem. 1994, 98, 8742.
(61) Wink, T.; de Beer, J.; Hennink, W. E.; Bult, A.; van Bennekom, W. P. Anal. Chem. 1999, 71, 801.
(62) Ron, H.; Matlis, S.; Rubinstein, I. Langmuir 1998, 14, 1116.
(63) Petrovykh, D. Y.; Kimura-Suda, H.; Whitman, L. J.; Tarlov, M. J. J. Am. Chem. Soc. 2003, 125, 5219.
(64) Sun, F.; Grainger, D. W.; Castner, D. G.; Leach-Scampavia, D. K. Macromolecules 1994, 27, 3053.
(65) Cavic, B. A.; McGovern, M. E.; Nisman, R.; Thompson, M. Analyst 2001, 126, 485.
(66) Barber, M.; Clark, D. T. Chem. Commun. 1970, 24.
(67) Castner, D. G.; Hinds, K.; Grainger, D. W. Langmuir 1996, 12, 5083.
(68) Bard, A. J.; Faulkner, L. R. Electrochemical Methods: Fundamentals and Applications; 2nd ed.; Wiley & Sons, Inc.: New York, 2000; p. 373.
(69) Rogers, Y.-H.; Jiang-Baucom, P.; Huang, Z.-J.; Bogdanov, V.; Anderson, S.; Boyce-Jacino, M. T. Anal. Biochem. 1999, 266, 23.
(70) Lenigk, R.; Carles, M.; Ip, N. Y.; Sucher, N. J. Langmuir 2001, 17, 2497.
(71) Fadley, C. S. Prog. Surf Sci. 1984, 16, 275.
(72) Bain, C. D.; Whitesides, G. M. J. Phys. Chem. 1989, 93, 1670.
(73) Meselson, M.; Stahl, F. W. Proc. Natl. Acad. Sci. USA 1958, 44, 671.
(74) Walsh, M. K.; Wang, X.; Weimer, B. C. J. Biochem. Biophys. Methods 2001, 47, 221.
(75) Steel, A. B.; Herne, T. M.; Tarlov, M. J. Anal. Chem. 1998, 70, 4670.
(76) Podyminogin, M. A.; Lukhtanov, E. A.; Reed, M. W. Nucl. Acids Res. 2001, 29, 5090.
(77) Beattie, W. G.; Meng, L.; Turner, S. L.; Varma, R. S.; Dao, D. D.; Beattie, K. L. Molec. Biotechnol. 1995, 4, 213.
(78) Tsao, M.-W.; Pfeifer, K.-H.; Rabolt, J. F.; Castner, D. G.; Haussling, L.; Ringsdorf, H. Macromolecules 1997, 30, 5913.

### EXAMPLE 5

### Polymer-Anchored DNA Gene Monolayers

Introduction. Monolayers of DNA chains of polymeric dimensions, considered here to be longer than ˜100 nucleotides, are widely encountered in biomolecular diagnostics as well as present a model system for investigating behavior of polyelectrolytes at interfaces. A major challenge in advancing such applications is assembling the DNA on the surface in a controlled way. Although covalent immobilization is expected to produce optimal stability, the multitude of potential reactive sites along the contour of long DNA molecules requires that any chemical transformations be strictly site-specific to preserve control over attachment geometry and function. A synthetic approach to fabricating monolayers of DNA genes on gold using polymeric anchor (adhesion) films is presented that (i) possesses stringent site-specificity of surface-attachment, (ii) exhibits excellent stability to elevated temperatures, allowing denaturation of duplex chains at 90° C. without loss of surface-linked strands, and (iii) achieves surface coverages suitable for investigating multi-chain polyelectrolyte behavior in regimes of strong interchain interactions.

The tremendous growth in biosensing and microarray technologies based on surface-tethered nucleic acids has heightened the urgency to fundamentally understand how nucleic acids behave at surfaces. From oligonucleotide film studies it is known that hybridization is suppressed relative to bulk solution.¹⁻³ Theoretical descriptions are also emerging to complement experimental observations.⁴⁻⁶ However, less scrutiny has been applied to monolayers of DNA chains that are truly macromolecular; designated herein as “polymeric” DNA, meaning chains of 100 monomers or longer. We have prepared and characterized model monolayers of polymeric DNA. These films have application in complementary DNA (cDNA) microarray technologies,⁷ DNA vaccines,⁸ and a spectrum of solid-phase molecular biology techniques.⁹⁻¹² They are also versatile experimental models for investigating surface-confined charged macromolecules, whose physicochemical behavior is often cast in terms of asymptotic laws reached at large chain lengths.¹³

Prevalent approaches for immobilizing long DNA fragments to surfaces are based on baking and UV crosslinking.¹⁴ These methods leave surface-bound molecules in a distribution of poorly understood conformations that likely involve multiple attachment points per chain. Site-specific, covalent attachment of polymeric DNA selectively via one terminus has been reported on glass⁹, agarose,¹² and gold,¹⁵ though stability limitations may constrain applications if regeneration (e.g. at elevated temperatures, as in PCR) of single-stranded molecules is required, or if long term trends need to be characterized. We therefore developed a methodology for gold supports combining stringent attachment via chain terminus with excellent stability, sufficient to withstand thermal denaturation at 90° C. without loss of surface-linked strands.

The approach herein generalizes a strategy based on “anchor films” of the polymer poly(mercaptopropyl)methyl siloxane (PMPMS).¹⁶ PMPMS forms a highly multidentate thiolate attachment to gold,^{16,17} and assembles in a nanometer-thin film that also provides a thiol-rich surface for further modification. Polyfunctional thiolate grafting is known to greatly enhance layer stability.¹⁸

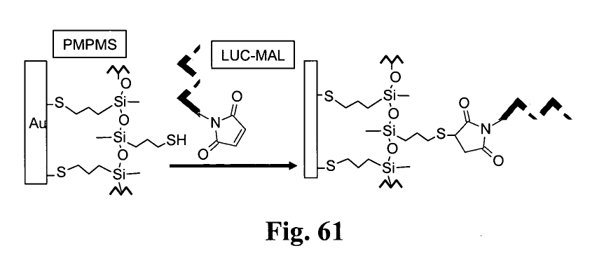

PMPMS Films Modified with LUC-MAL. PMPMS films were derivatized with 1943 base pair (bp) double-stranded DNA (dsDNA) chains. These molecules, prepared by PCR from plasmid precursors (pT7LUC, Promega), contained the gene for firefly luciferase (LUC; 1650 bp) under the control of a T7 promoter. Disulfide-modified primers were used to introduce a disulfide terminus to the genes which was subsequently reduced with dithiothreitol to liberate a terminal thiol, followed by reaction with bis-maleimidotetraethylene glycol (BMPEO4, Pierce Biotechnology) to yield maleimide-terminated gene constructs, “LUC-MAL.” A 100-fold excess of BMPEO4 to LUC amplicons was used in a 2 h reaction in pH 7.0, 0.015 M citrate buffer, 1 M NaCl (SSC1M buffer). LUC-MAL was immobilized on 2 to 3 nm thick PMPMS films from ˜1×10⁻⁸ M solutions in SSC1M (FIG. 61). Control “LUC” chains, without a reactive terminus, were prepared from unmodified primers of the same sequence.

Compared to immobilization of oligonucleotides, attachment of polymeric DNA exacerbates prospects of side-reactions because thousands of potentially competing reactive sites exist along the chain. For example, although aromatic amines on nucleic bases are not known to be highly reactive, there are ˜10,000 of these moieties in LUC-MAL. This excess, relative to a single endgroup, raises concern that a crosslinker like BMPEO4 may also react at internal positions leading to loss of control over final attachment geometry. To test for such side reactions we carried out expression of the LUC gene as a diagnostic screen (TnT® Wheat Germ Extract, Promega), reasoning that modifications within the gene should interfere with RNA polymerase during transcription and block synthesis of the firefly luciferase enzyme. Quantification of the level of expressed enzyme was accomplished by addition of beetle luciferin, which is digested by luciferase with concomitant emission of light. Luminescence from bulk solutions of LUC chains after the standard BMPEO4 treatment were within ±15% of those from untreated genes. The comparable gene activity suggests that little if any modification took place at internal DNA positions.

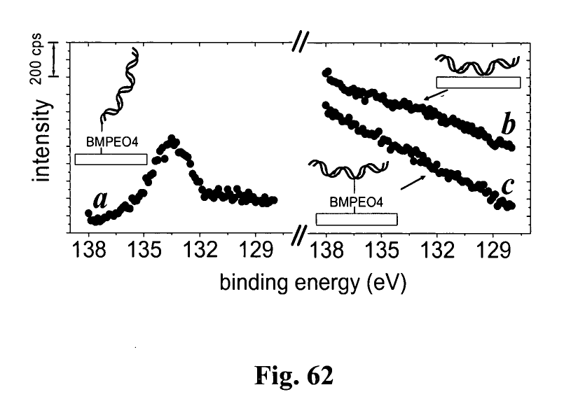

XPS was used to examine whether LUC-MAL attached to PMPMS site-specifically through one end. An end-tethered geometry is expected to be least interfering with hybridization or enzymatic addressing of the immobilized molecules. FIG. 62, trace a, shows P 2p photoelectron emissions after attachment of LUC-MAL. The integrated intensity of this trace translates to a surface coverage of 4.1×10¹⁰ chains/cm². This coverage, calculated from absolute intensity using an independently measured instrument response factor, actually represents a lower limit because the calculation assumed zero attenuation of P 2p intensity. As described in Supporting Information, true coverage is estimated to be higher by up to 30%. Sample b is a control for physical adsorption; these strands were not end-modified or treated with BMPEO4. Trace c is a control for immobilization through internal sites; these strands did not carry a terminal disulfide but otherwise underwent the standard BMPEO4 treatment. The absence of P 2p emission in samples b and c indicates that, within sensitivity of the XPS (˜5×10⁹ chains/cm²) attachment of LUC-MAL is site-specific.

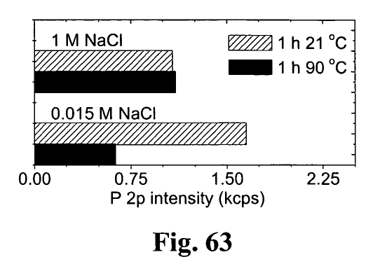

The ability to denature bound chains without loss of surface-linked strands is essential to applications in nucleic acid diagnostics and solid phase PCR protocols.^{9,10,19} FIG. 63 shows changes in P 2p intensity after immersion of LUC-MAL monolayers for 1 h at 90° C. in citrate buffers at two ionic strengths, 1.0 M and 0.015 M NaCl. The high ionic strength data reveal excellent stability, with no loss of DNA evident, in agreement with earlier data on oligonucleotide films.¹⁶ In contrast, direct chemisorption of DNA to gold through a single thiol fails under similarly aggressive conditions.¹⁶ At the lower ionic strength, a ˜60% decrease of P 2p signal is observed. This decrease is attributed to melting of the double helix. The lower added salt is insufficient to screen strand-strand electrostatic repulsions, causing loss of the strand not covalently linked to the solid support. The observed decrease does not exactly equal the expected value of 50%. The cause for this is not clear but is suspected to arise from different arrangements of immobilized single-stranded DNA (ssDNA) and dsDNA that alter attenuation of P 2p emission within these films.

The melting temperature T_{M} of polymeric dsDNA is estimated from:²⁰
T_{M}=81.5° C.+16.6 log M+0.41(% GC)−500/n
M is monovalent salt (mol/L), % GC is percentage of GC base pairs, and n is duplex length. For LUC-MAL, n=1943 bp and % GC=45%, leading to T_{M}=100° C. in 1 M NaCl and 69° C. in 0.015 M NaCl. The calculated T_{M} values agree with the data reported in FIG. 63, namely stability under 1 M salt but dissociation of the strands when ionic strength decreases to 0.015 M. Melting of the dsDNA is also additional evidence that it is not internally crosslinked by BMPEO4, since crosslinking would have suppressed strand separation.

The behavior of interfacial polymeric systems is in large part governed by chain-chain interactions, which are intensified due to “crowding” of the chains at the surface. A quantitative measure of crowding is obtained by defining an “overlap density” σ^{O},^{21,22}
σ^{O}=1/(πR_{g}²) R_{g}²=pL/3
R_{g} is the polymer's radius of gyration, p its persistence length, and L the contour length. For surface densities above σ^{O} chains are expected to come into physical contact. For LUC chains, L=1943 bp×0.34 nm/bp=660 nm and, under moderate to high ionic strengths, p≈50 nm²³ Therefore, σ^{O}≈3×10⁹ chains/cm². The highest surface densities realized in our studies were for 60 h attachment. At these long times, hydrolysis of the maleimide function limited further increase in immobilized DNA. The 60 h XPS-derived coverage was 6.1×10¹⁰ chains/cm², about 20 times the overlap threshold. Therefore, the chains will interact and their behavior will be collective rather than representative of isolated molecules. Importantly, the overall rigidity of LUC dsDNA is fairly high as it only contains ˜13 persistence lengths. Chain rigidity and thus conformational statistics are adjusted by controlling the ratio L/p. Systematic variations of this parameter allow examining the crossover behaviors between rodlike and flexible polyelectrolytes tethered at surfaces.

### REFENCES FOR EXAMPLE 5

(1) Peterson, A. W.; Wolf, L. K.; Georgiadis, R. M. J. Am. Chem. Soc. 2002, 124, 14601-14607.
(2) Watterson, J. H.; Piunno, P. A. E.; Wust, C. C.; Krull, U. J. Langmuir 2000, 16, 4984-4992.
(3) Steel, A. B.; Herne, T. M.; Tarlov, M. J. Anal. Chem. 1998, 70, 4670-4677.
(4) Vainrub, A.; Pettitt, B. M. J. Am. Chem. Soc. 2003, 125, 7798-7799.
(5) Halperin, A.; Buhot, A.; Zhulina, E. B. Biophys. J. 2004, 86, 718-730.
(6) Chan, V.; Graves, D. J.; Mckenzie, S. E. Biophys. J. 1995, 69, 2243-2255.
(7) Ramsay, G. Nat. Biotechnol. 1998, 16, 40-44.
(8) Fynan, E. F.; Webster, R. G.; Fuller, D. H.; Haynes, J. R.; Santoro, J. C.; Robinson, H. L. Proc. Natl. Acad. Sci. USA 1993, 90, 11478-11482.
(9) Andreadis, J. D.; Chrisey, L. A. Nucl. Acids Res. 2000, 28, e5.
(10) Camon, A.; Vision, T. J.; Mitchell, S. E.; Thannhauser, T. W.; Muller, U.; Kresovich, S. Biotechniques 2002, 32, 410-420.
(11) Shivashankar, G. V.; Liu, S.; Libchaber, A. Appl. Phys. Lett. 2000, 76, 3638-3640.
(12) Marble, H. A.; Davis, R. H. Biotechnol. Prog. 1995, 11, 393-396.
(13) Netz, R. R.; Andelman, D. Physics Reports 2003, 380, 1-95.
(14) Nierzwicki-Bauer, S. A.; Gebhardt, J. S.; Linkkila, L.; Walsh, K. Biotechniques 1990, 9, 472-478.
(15) Yang, M. S.; Yau, H. C. M.; Chan, H. L. Langmuir 1998, 14, 6121-6129.
(16) Johnson, P. A.; Levicky, R. Langmuir 2003, 19, 10288-10294.
(17) Sun, F.; Grainger, D. W.; Castner, D. G.; Leach-Scampavia, D. K. Macromolecules 1994, 27, 3053-3062.
(18) Sun, F.; Castner, D. G.; Grainger, D. W. Langmuir 1993, 9, 3200-3207.
(19) Oroskar, A. A.; Rasmussen, S. E.; Rasmussen, H. N.; Rasmussen, S. R.; Sullivan, B. M.; Johansson, A. Clin. Chem. 1996, 42, 1547-1555.
(20) Meinkoth, J.; Wahl, G. Anal. Biochem. 1984, 138, 267-284.
(21) Alexander, S. J. de Physique 1977, 38, 983-987.
(22) de Gennes, P.-G. Macromolecules 1980, 13, 1069-1075.
(23) Bloomfield, V. A.; Crothers, D. M.; I Tinoco Jr. Nucleic Acids—Structures, Properties, and Functions; University Science Books: Sausalito, 2000; 417-418.

Supporting Information.

Materials. Poly(mercaptopropyl)methylsiloxane (PMPMS, degree of polymerization ˜40) was purchased from Gelest Inc., dithiothreitol (DTT) from Fisher Scientific, and the crosslinker bis-maleimidotetraethylene glycol (BMPEO4) and Ellman's reagent (5,5′-dithio-bis-(2-nitrobenzoic acid)) were from Pierce Biotechnology. Oligonucleotides were from Qiagen Operon and included purification by HPLC. Three oligonucleotide sequences were used: a disulfide-terminated sequence 5′ HO(CH₂)₆—S—S—(CH₂)₆-CAA TAC GCA AAC CGC CTC TCC 3′ (P1-S-SR) (SEQ. ID. NO. 4) and unmodified sequences 5′ CAA TAC GCA AAC CGC CTC TCC 3′ (P1) (SEQ. ID. NO. 4) and 5′ TCG GTG ATG TCG GCG ATA TAG G 3′ (P2) (SEQ. ID. NO. 5). TnT® wheat germ extract system for coupled transcription/translation was from Promega. Buffers were prepared using 18.4 MΩ cm water from a Millipore Biocell purification system.

Linear, 1943 bp long double-stranded (dsDNA) molecules were prepared by PCR amplification from plasmid precursors (pT7LUC, Promega). These chains consisted of a 64 bp spacer sequence in front of a T7 promoter, the promoter sequence, the firefly luciferase gene of 1650 bp, a poly(A) termination region, and a 63 bp tail. Chains without a terminal modification, referred to as “LUC” chains, were amplified using primers P1 and P2. Disulfide-terminated chains, “LUC-S-SR”, were prepared using P1-S-SR and P2 primers. The disulfide was introduced to the promoter end of the gene. The PCR cocktail consisted of 1×PCR Master Mix (Promega), 1 μM concentrations of each primer, 0.5 ng of pT7LUC DNA template, and nuclease-free water. PCR settings (Eppendorf Mastercycler) included an initial ramp to 95° C. for 3 minutes, 30 amplification cycles of [94° C., 45 sec; 57.3° C., 45 sec; 72° C., 2 min], and a final extension cycle at 72° C. for 7 minutes. Samples were then held at 4° C. until collected. LUC chains were purified on QIAquick PCR Purification spin columns (Qiagen) following manufacturer instructions. Their monodispersity was confirmed via agarose gel electrophoresis (E-C Minicell) using 0.5 μg/ml ethidium bromide stain and visualization under UV light. LUC-S-SR PCR product was processed as described below. DNA concentrations were determined from A₂₆₀ absorption measurements on a Cary 50 spectrophotometer.

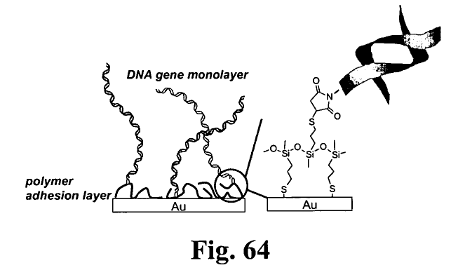

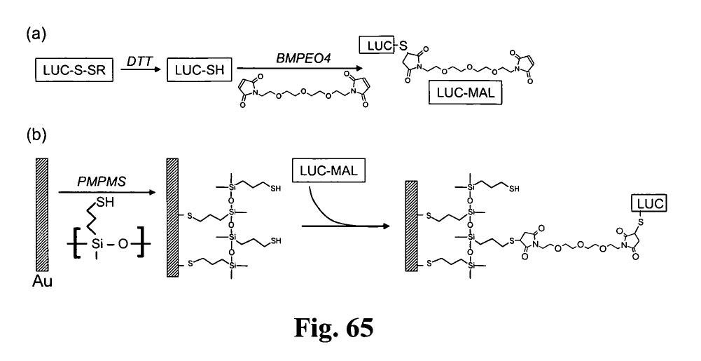

FIG. 64 illustrates attachment of DNA gene monolayer to a PMPMS polymer adhesion layer. dsDNA constructs with a terminal maleimide moiety, “LUC-MAL”, were prepared from LUC-S-SR precursors. A 500-fold excess of DTT in 100 μL of SSC1M (SSC1M: 0.015 M sodium citrate, 1 M NaCl, pH 7.0) to total disulfide was added to 700 μL of crude PCR product, and allowed to react for 1 h to reduce disulfide to thiol groups, producing “LUC-SH”. The LUC-SH mixture was loaded on QlAquick spin columns, twice washed according to manufacturer instructions, and eluted with a 1×10⁻⁴ M sodium citrate pH 7.5 elution buffer. The elution buffer differed from that recommended in order to avoid amine groups in the eluent, which could potentially react with crosslinker imides in subsequent steps¹. Typical recovery was 400 μL containing ˜3×10⁻⁸ M LUC-SH. The eluent was adjusted to 1M NaCl. BMPEO4 at a concentration of 2.8×10⁻³ M in SSC1M was added to a 100-fold excess over LUC-SH and, after a 2 h reaction, LUC-MAL was purified by passage through NAP-5 columns (Amersham Biosciences) with SSC1M as elution buffer. 750 μL of recovered LUC-MAL, at a typical concentration of 1×10⁻⁸ M in SSC1M, was immediately used to derivatize PMPMS-coated supports. FIG. 65 illustrates the above sequence of chemical steps.

Sample Preparation. Glass slides were cleaned by immersion in hot “piranha” (70/30 mixture of conc. H₂SO₄ and 30% aqueous solution of H₂O₂) for a minimum of 20 minutes, rinsed thoroughly with deionized water, and dried with a nitrogen stream. WARNING: piranha solution is extremely oxidizing and should never be stored in tightly capped containers on account of gas evolution. Cleaned slides were coated with a 20 nm Cr adhesion sublayer and a 300 nm Au toplayer by thermal evaporation. Just prior to use, gold surfaces were cleaned for 20 minutes in a UV-ozone cleaner system (Jelight Company, Model 342) followed by 30 minutes immersion in ethanol to reduce any gold oxide that may have formed². The surfaces were washed with toluene and transferred, without drying, into 1 mM (monomer residues/volume) PMPMS solutions in toluene. Typical immersion times were 1 h. Following adsorption of PMPMS, slides were rinsed with toluene, dried under a nitrogen stream, and used immediately for DNA immobilization.

For DNA attachment a silicone gasket with cut-out “reaction” wells was sandwiched between a PMPMS-modified and a plain slide to create a set of circular chambers 0.54 cm² in area. DNA solutions in SSC1M were introduced by needle and syringe through the gasket into the sealed wells. After a designated time, the wells were drained and rinsed with SSC1M buffer four times while still sealed, disassembled, and subjected to a final rinse with deionized water. The surfaces were then dried and characterized by XPS (see below).

The time available for immobilization of LUC-MAL is limited by hydrolysis of the maleimide function. The hydrolysis rate was determined by using mercaptoethanol to titrate solutions of 1.7×10⁻⁴ M BMPEO4 in 0.01 M pH 7.2 buffer, after various times of storage. Decrease in mercaptoethanol concentration due to consumption by unhydrolyzed BMPEO4 maleimides was quantified spectrophotometrically using Ellman's reagent following provider instructions. Ellman's reagent undergoes an exchange reaction with free mercaptoethanol to produce the strongly absorbing species 2-nitro-5-thiobenzoic acid, which is detected at 412 nm and correlated with mercaptoethanol concentration through a calibration curve. About 50% of maleimide groups hydrolyzed within 24 h, after 72 h only 20% of maleimides remained active, and after 7 days no active maleimides were detected.

XPS Characterization and Analysis. XPS measurements were performed on a Physical Electronics PHI 5500 instrument equipped with an Al X-ray monochromatic source (Al Kα line, 1486.6 eV) and a spherical capacitor energy analyzer. Elemental scans were carried out for gold (Au 4f), carbon (C 1s), silicon (Si 2p), oxygen (O 1s), sulfur (S 2p), phosphorus (P 2p), and nitrogen (N 1s) at a 45° takeoff angle. Typical integration times were 3 minutes for Au, 6 minutes for C and O, 15 minutes for Si, S, and N, and 60 minutes for P. Elemental detection limits were approximately 0.1% of total photoelectron intensity. XPS traces were baseline corrected with the program XPSPeak, with baselines modeled as a combination of Shirley and linear functions.

XPS data were analyzed to derive DNA surface coverage using equation (5.1),
Iₘ=R(θ)ₘrσₘXₘ/sin θ (5.1)
Iₘ is the integrated signal intensity from a monolayer of atoms, R(θ)ₘ is instrument response function at takeoff angle θ for spectral line m, σₘ is surface density of emitting atoms (atoms/true area), r is a roughness-correction factor (ratio of true to geometric area; r≧1), and Xₘ is the differential photoionization cross-section. The product rσₘ is the surface density of atoms as seen by the instrument's analyzer, and is written as such in equation (5.1) to emphasize that the reported DNA coverage values are on a per geometric area basis. The takeoff (grazing) angle θ is defined between path of detected photoelectrons and the sample surface. The intensity Iₘ refers to baseline-corrected peak area. Details of derivation of equation (5.1) are available in the literature³. As further discussed below, we note that equation (5.1) assumes that the emitted intensity Iₘ is not attenuated by the presence of an overlayer.

LUC-MAL coverages (per geometric area) were estimated from absolute P 2p intensities by calculating the phosphorus atom coverage,
rσ_{P}=I_{P2p} sin θ/R(θ)_{P2p}X_{P2p} (5.1b) and
dividing rσ_{P} by 3886, the number of P atoms per LUC chain. X_{P2p}=1290 barns was taken from Scofield's tables⁴. The instrumental response function R_{P2p} (at 133 eV) was interpolated from R_{Si2p} (at 100 eV) and R_{Si2s} (at 151 eV). Values of R at the two silicon lines were measured from fused silica reference slides cleaned in situ with a beam of Ar ions until C 1s emission was negligible, and using equation (5.2) to calculate R³
R(θ)_{Si}=I_{Si}/ρ_{Si}X_{Si}Λ_{Si}^{S} (5.2)

The number density of silicon atoms in fused silica ρ_{Si}=2.2×10²² atoms/cm³, the attenuation lengths of photoelectrons in the silica support Λ_{Si2s}^{S}=3.4 nm and Λ_{Si2p}^{S}=3.5 nm⁵, and the cross-sections X_{Si2s}=1030 barns and X_{Si2p}=884 barns were taken from Scofield⁴. R changed by less than 3% over the range of interpolation.

Equation (5.1b) assumes that P 2p emission from DNA films was not attenuated. This assumption was partially motivated by the expectation that the DNA chains are the topmost layer of the sample. Nevertheless, experimental P 2p emissions are expected to be attenuated as they need to pass through the DNA layer itself. The expected error resulting from the assumption of no attenuation is calculated as follows. For a full DNA monolayer in which the chains lie side-by-side as aligned horizontal cylinders that completely cover the surface, the film thickness would be t≈2 nm (diameter of dsDNA), and the density of phosphorus atoms ρ_{P}=#P/h A≈2/(0.34×4)=1.5 nm⁻³. Here, the number of P atoms (#P) is 2 per volume hA, where h is the length of one base pair (0.34 nm) along the cylinder axis and A≈(DNA diameter)²=4 nm² is the cross-sectional area of the film attributed to one chain. For such a full monolayer, the corresponding surface density of P atoms is σ_{P}=2/(0.34×2)=2.9 nm⁻². In estimating the error due to attenuation one also needs the attenuation length of P 2p photoelectrons in the DNA layer, Λ_{P2p}^{O}, which is taken as 3.9 nm from experiments of Inagaki et al⁶ as tabulated in Tanuma et al⁷. With these values, the P 2p intensity from a full DNA monolayer, including effects of attenuation, are calculated from³
I_{P2p}=R(θ)_{P2p}ρ_{P}X_{P2p}Λ_{P2p}^{O}(1−exp[−t/Λ_{P2p}^{O} sin θ]) (5.3)

Comparison of values calculated from equations (5.3) and (5.1) for θ=45° predicts that, for a full monolayer of dsDNA with ρ_{P}=1.5 mm⁻³, the intensity would be attenuated by 28% compared to intensity from the equivalent coverage of P atoms (σ_{P}=2.9 nm⁻²) but without attenuation. In other words, neglecting corrections for signal decrease due to attenuation would result in an underestimate of DNA coverage by ˜30%. Our experimental samples, allowing for a 30% increase in coverage, approach those of a complete monolayer of horizontal cylinders considered above. Thus multiplication of calculated values from equation (5.1) by 1.3 provides values close to those that would have been estimated from equation (5.3).

For reporting coverage values we assume no attenuation in equation (5.1). This assumption translates to a lower limit on coverage, since some attenuation is always expected. In contrast, equation (5.3) assumes homogeneous, planar, uniformly thick DNA films. The impact of these assumptions on calculated coverage is more ambiguous; for example, the DNA chains might pile over one another, and there may be effects due to roughness and voids that are difficult to assess without more detailed information on sample structure.

Luciferase Expression Measurements. The ability of RNA polymerase to produce active luciferase enzyme by transcribing LUC dsDNA was used as a diagnostic for potential side reactions with BMPEO4. 4 μL of solution containing LUC DNA at a concentration of 31 μg/ml (2.6×10⁻⁸ M) in 1×SSC buffer (0.015 M sodium citrate, 0.15 M NaCl, pH 7.0) was added to 96 μL of TnT® wheat germ extract mixture (Promega) prepared according to manufacturer instructions. After 2 h of incubation at 30° C. to allow synthesis of the luciferase enzyme, 5 μL of extract were withdrawn and added to 50 μL of assay solution (Promega) containing luciferin. Intensity of the resultant chemiluminescence was measured on a Lumat LB9501 luminometer using a 12 sec integration time.

LUC chains used in the luminescence assays were prepared using primers P1 and P2. The disulfide end-group was deliberately left off to avoid conjugation with species present in the transcription/translation solution. The PCR dsDNA product was purified on QIAquick spin columns (Qiagen) using 0.1 mM pH 7.5 sodium citrate buffer for elution. The eluent was adjusted to 1 M NaCl strength by addition of 20×SSC. One aliquot (untreated LUC) was set aside, while to a second aliquot (treated LUC) was added 1 mg/ml (2.8×10⁻³ M) BMPEO4 in SSC1M to produce a 100-fold excess over LUC chains. This aliquot was kept 2 h under the BMPEO4 solution before purification by a second pass through QlAquick spin columns. Both aliquots were adjusted to a final concentration of 31 μg/ml in 1 M NaCl citrate buffer before performing the luciferase assay.

### REFERENCES FOR SUPPORTING INFORMATION

(1) Smyth, D. G.; Blumenfeld, O. O.; Konigsberg, W. Biochem. J. 1964, 91, 589-595.
(2) Ron, H.; Matlis, S.; Rubinstein, I. Langmuir 1998, 14, 1116-1121.
(3) Fadley, C. S. Prog. Surf. Sci. 1984, 16, 275-388.
(4) Scofield, J. H. J. Electron. Spectrosc. 1976, 8, 129-137.
(5) Powell, C. J.; Jablonski, A. NIST Electron Effective-Attenuation-Length Database—Version 1.0; National Institute of Standards and Technology: Gaithersburg, Md., 2001.
(6) Inagaki, T.; Hamm, R. N.; Arakawa, E. T.; Painter, L. R. J. Chem. Phys. 1974, 61, 4246-4250.
(7) Tanuma, S.; Powell, C. J.; Penn, D. R. Surf. Interface Anal. 1993, 21, 165-176.

### EXAMPLE 6

### Organization and Hybridization Activity of Oligodeoxyribonucleotides on Maleimide-Functional Solid Supports

Introduction. Nucleic acid molecules, immobilized on solid supports, find widespread applications in genotyping and gene expression analysis and in the related area of biosensors. It is well recognized that hybridization at an interface is different from that under bulk conditions. In general, surface interactions, as well as interactions between immobilized neighbors, are expected to influence activation barriers to hybridization as well as overall thermodynamics of binding between a strand attached to a solid support and a complementary sequence in bulk solution. Since a solid surface functionalized with nucleic acid molecules is electrically charged, space-charge regions akin to the electrostatic double layer develop in which concentrations of small mobile ions (counterions, salt ions) at the interface are different from those in bulk solution. For these reasons, physical aspects of hybridization under the local surface conditions are expected to fundamentally differ from those in bulk solution.

The importance of understanding behavior of surface-tethered nucleic acid films has stimulated a number of physically-oriented studies. Work by Tarlov and coworkers, by the Georgiadis and Corn groups, and by others on oligonucleotide layers on metal supports have begun to document influence of key parameters on organization and hybridization of immobilized nucleic acids. These studies are underpinned by the ability to prepare precisely-controlled oligonucleotide layers on gold supports, using thiol-mediated tethering of nucleic acids to the support. Gold supports exhibit crystalline facets whose chemistry is readily tailored using alkanethiol molecules that organize in dense, well-ordered patterns reflective of the underlying order of the support. A broad range of surface modifications may be introduced, for example to exert control over DNA-surface interactions. Significantly, a spectrum of measurement techniques agree that close to 100% activity, or hybridization efficiency, of probe molecules' ability to bind perfectly matched target complements may be achieved under suitable conditions. Usually, this requires oligonucleotide probe coverages of ˜5×10¹² probes/cm² or lower to mitigate electrosteric repulsions encountered by incoming target sequences [1][2][3][4][5][6]. In this Example, “probes” refer to immobilized strands while “targets” denote strands in bulk solution.

In contrast, immobilization of nucleic acids on siliceous surfaces such as glass and silica, arguably the most common type of solid support, must address their more complex surface chemistry. The reactive sites on siliceous surfaces are silanol (—Si—OH) groups, which exhibit various reactivities depending on hydrogen bonding state and connection to the skeletal silica matrix. Additional complexity results from application of organosilanes as surface modification agents, aminosilanes and mercaptosilanes being the most common. These reagents bring additional complexity due to their ability to cross-polymerize as well as to bind covalently to the support, processes that are moreover sensitive to water content levels and to catalytic action, for example by amines. Despite these intricacies, the widespread use of siliceous supports motivates thorough investigations of protocols developed for their modification with nucleic acids. While applications require that probe surfaces behave reproducibly, fundamental insight also requires understanding why certain probes on a slide will hybridize while others will not. We investigated the use of heterobifunctional linkers incorporating maleimide functions to tether oligonucleotides to siliceous surfaces. This Example investigates hybridization activity of probe molecules immobilized using maleimide-based surface conjugation chemistry. Maleimides react with thiol and amine groups, and have been used for preparing bio-derivatized surfaces by a number of investigators.

Materials and Methods

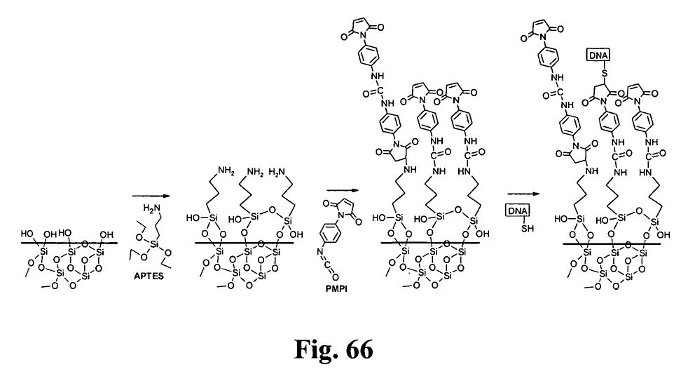

Attachment Overview. Attachment of thiol-terminated oligonucleotides to siliceous supports was accomplished in three steps, illustrated in FIG. 66. Silica or glass supports were first modified with (aminopropyl) triethoxysilane (APTES; 98% purity; Aldrich), followed by a second step in which the heterobifunctional crosslinker p-maleimidophenyl isocyanate (PMPI, Pierce Biotechnology) reacts with APTES through the PMPI isocyanate group to form an urea linkage, and a final step in which sulfhydryl-terminated single-stranded oligonucleotides immobilize by addition of the sulfhydryl moiety to the PMPI maleimide C═C bond. Exceptions to this overall scheme arise, however. For example, on account of side reactions between PMPI's isocyanate group and trace water, bismaleimide compounds may form. The bismaleimides covalently immobilize by maleimide-amine coupling, as also indicated in FIG. 66. Although such side reactions do not change the surface coverage of maleimide groups, they do introduce an apparent excess of PMPI residues as probed, for example, by infrared spectroscopy.

Modification of Fumed Silica Supports with APTES and PMPI. Aerosil® 200 fumed silica powder (manufacturer provided specific surface area: 200 m²/g; purity: 99.8% amorphous SiO₂; supplier: Degussa-Hüls) was used to examine steps 1 and 2 of the modification protocol of FIG. 66. The specific surface area was independently confirmed using BET analysis by Micromeritics Instrument Corp. The large specific area enabled characterization of modified silica powders by infrared spectroscopy and titration analysis, so that chemical state of the organically-modified surface could be assessed. Silica supports bearing only an APTES monolayer will be denoted as silica/APTES, while silica/APTES/PMPI will signify supports having undergone both APTES and PMPI modification.

Silica/APTES and silica/APTES/PMPI samples were prepared as described previously. Briefly, as-received silica was vortexed in a 1% w/w (40 mM) solution of APTES in anhydrous toluene for 30 minutes at room temperature. The modified powder was centrifuged and subjected to successive washes by repetitively mechanically dispersing it in fresh solvent, centrifuging, and decanting. Two toluene washes, one deionized water (Millipore Biocell) wash, and one acetonitrile wash were used. The washed silica/APTES support was dried overnight at 100° C. inside a kiln. To prepare silica/APTES/PMPI samples, silica/APTES powder, typically 20 to 80 mg, was weighed into polypropylene vials and PMPI crosslinker was added in anhydrous acetonitrile. The amount of PMPI was such that NNN PMPI molecules were added per nm² of powder surface (PMPI concentration ˜50 mM). Less was used if incomplete conversion of the silica/APTES surface was desired. The reaction was carried out for 30 minutes at room temperature while vortexing. The powder was centrifuged and decanted, followed by two washes with acetonitrile, one with saline phosphate buffer (PBS: 10 mM sodium phosphate, 1 M NaCl, pH 7), and a third acetonitrile wash. The powder was dried overnight at 50° C.

Maleimide Surface Reactivity and Stability. Transmission infrared spectroscopy and Ellman's titration were used to monitor reactivity and chemical transformations of fumed silica powders. Infrared spectra were collected by sandwiching 3 mg of powder within a cardboard gasket sealed between two calcium fluoride windows. 1000 scans were averaged at 4 cm⁻¹ resolution, using a Nicolet Magna 560 spectrometer with a liquid nitrogen cooled MCT detector. Care was taken to ensure a uniform distribution of powder across the infrared beam. From calibrations against independently measured APTES and PMPI coverages, determined via elemental analysis, the following equations were derived to allow calculation of APTES or PMPI coverage from integrated IR absorbances:

$$\begin{matrix}APTES ( residues \text{/} {nm}^{2} ) = \frac{1}{1.89} \frac{{\int }_{2800}^{3000} A ( v ) ⅆ v}{{\int }_{1820}^{ 1920} A ( v ) ⅆ v} & ( 6.1 ) \\ PMPI ( residues \text{/} {nm}^{2} ) = \frac{1}{3.25} \frac{{\int }_{1363}^{1430} A ( v ) ⅆ v}{{\int }_{1820}^{ 1920} A ( v ) ⅆ v} & ( 6.2 )\end{matrix}$$

Equation (6.2) involves absorptions that change with reaction of the PMPI residue; therefore, it is only applicable to freshly made supports.

Preparation of DNA-Modified Glass Slides. Glass slides were cleaned for 15 minutes in a UV-ozone cleaner (Model T10X10/OES, UVOCS Inc.) followed by immersion in deionized (DI) 18.4 MΩ cm water, then toluene, and finally anhydrous toluene. Thus cleaned slides were placed in glass bottles containing 2.8 mM solution of APTES in anhydrous toluene for 30 minutes. Afterwards the slides were successively washed twice in toluene, once with DI water, and once with acetonitrile. The duration of each wash was 10 minutes.

A pair of APTES-modified slides was used to sandwich a silicone gasket (Grace BioLabs) to create a sealed chamber. The chamber was then filled with a 2.3 mM solution of PMPI in anhydrous acetonitrile for 2 hours. The PMPI solution was injected through the silicone gasket using needle and syringe. The chamber was sealed to the external atmosphere during reaction. After surface modification with PMPI was complete, a syringe was used to drain the cell and to refill it with pure acetonitrile for 10 minutes. A second 10-minute wash followed using 0.015 M sodium citrate, 1 M NaCl, pH 7.0 buffer (SSC1M buffer). Afterwards, the buffer was drained and probe solutions in SSC1M were introduced. All of these steps were carried out without disassembly of the cell in order to minimize potential for surface contamination. Table 6.1 shows the sequences and modifications of the various oligomers employed in this study, including the probe sequence P1 and control P2. All were purchased from Qiagen Inc. and included purification by HPLC. Thiol-terminated P1 was generated from as-received disulfide-terminated precursors by cleaving the disulfide with 200-fold excess of dithiothreitol (Pierce Biotechnology) in PBS for 1 hour, followed by purification on PD-10 columns (Amersham Biosciences). Purified P1 probes were used immediately for attachment to PMPI-activated glass slides, using 1 μM concentration in PBS and immobilization times from 2 h to 5 days. After attachment of probe, slides were washed by draining the probe solution and briefly refilling the reaction cell 4 times with pure SSC1M, followed by a fifth refill for 10 minutes before disassembly of the chamber, washing with DI water to remove salts, and drying under a nitrogen flow. The dried slides were used directly for characterization or for hybridization assays.

TABLE 6.1
Oligonucleotide Sequences

| abbrevi- | | |
|---|---|---|
| ation | Sequence (5′ to 3′) | notes |
| P1 | CGT TGT AAA ACG ACG GCC AG-(CH₂)₃SH | 3′ thiol-modified probe |
| | (SEQ ID NO. 1) | |
| P2 | CGT TGT AAA ACG ACG GCC AG | unmodified probe (control) |
| | (SEQ ID NO. 1) | |
| TC | CTG GCC GTC GTT TTA CAA CG 6FI˜Q | complementary target to P1 |
| | (SEQ ID NO. 2) | |
| TNC | CTA ACT GTT ACC TCG GTC GG 6FI˜Q | noncomplementary target |
| | (SEQ ID NO. 3) | (control) |

The unmodified P2 probe, without a sulfhydryl group, served as control to demonstrate effect of a terminal sulfhydryl on probe immobilization. TC and TNC oligomers were employed in hybridization assays, described below.

Oligonucleotide Attachment and Hybridization Activity. DNA coverages were calculated from absolute intensity of P 2p emission by independently determining the instrumental response function R_{P2p} and then solving equation (6.3) for the phosphorus coverage σ_{P} (atoms/cm²). From σ_{P}, surface density of DNA strands was calculated taking 20 P atoms per strand. In applying equation (6.3) it was assumed that no covering overlayer was present (t=0); in other words, that the DNA strands formed the topmost layer so that σ_{P}=I_{P2p} sin θ/R_{P2p}X_{P2p} (Equation 6.3). I_{P2p} was the integrated area of the P 2p band, X_{P2p} was taken from Scofield's tables [7], and R_{P2p} was interpolated from R_{Si2p} and R_{Si2s} measured from a fused silica reference slide. The silica slide was cleaned in situ with a beam of Ar ions until C 1s emission was negligible; for such a clean surface, the response function could be calculated directly from equation (6.1) taking t=0 (no overlayer) and given the silica density (2.2 g/cm³), composition (SiO₂), and attenuation lengths calculated from the NIST Electron Effective-Attenuation-Length Database [8] (Λ_{Si2s}^{S}=3.4 nm, Λ_{Si2p}^{S}=3.5 nm). Provided that identical instrumental settings are used, the Si 2p and 2s emissions at 151 and 100 eV provide values for R which may be interpolated to that for P 2p emission at 133 eV. As R changed by less than 4% over the range of interpolation, the principal source of error in calculation of DNA coverage is expected to arise from experimental uncertainty in I_{P2p}.

Target oligonucleotides were also fluorescently modified to allow qualitative confirmation of sequence-specificity of hybridization using bulk solution fluorometry.

Results and Discussion

Stability of maleimide-functional supports. The olefinic double bond of maleimides, typically exploited for conjugation of sulfhydryl groups to yield thioether linkages, is also reactive toward other nucleophiles. Unprotonated primary and aromatic amines are known to add readily, and under aqueous environments reaction with hydroxyl anions is a possibility. When using maleimide-functional supports for surface-immobilization of nucleic acids it is important to consider to what extent such side reactions influence conformation of immobilized strands and the ultimate chemical constitution of the support.

High surface area silica powder (Aerosil® 200), with a specific surface area of 200 m²/g, was silylated with APTES in toluene followed by reaction with PMPI in acetonitrile to introduce surface maleimide groups. The large specific surface area makes these powder samples well suited to characterization by conventional techniques of infrared spectroscopy and titration. Surface coverages of APTES and PMPI were estimated from equations (6.1) and (6.2), respectively. Recently, it was noted that maleimide groups are prone to reactions with immobilized aminosilanes. When the intention is to modify a support with a linker while preserving the linker's maleimide activity for subsequent immobilization of biomolecules, it is necessary to employ sufficiently aggressive conditions to ensure near complete conversion of the silane amine during linker immobilization. Otherwise, remnant amine groups will participate in a secondary reaction with linker maleimides, leading to deactivation of the support toward attachment of biomolecules. Indeed, one motivation for using PMPI, a heterobifunctional crosslinker based on isocyanate and maleimide functions, as opposed to more familiar crosslinkers based on NHS-ester and maleimide moieties, is that the highly reactive and sterically accessible isocyanate group facilitates near complete conversion of the APTES amines. Thus, undesirable reactions between the linker maleimide moiety and a silane amine are mitigated.

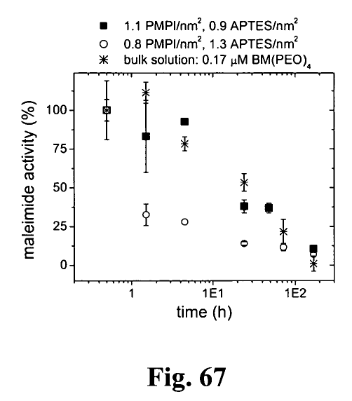

Ellman's analysis, in which maleimide activity is monitored through capacity of the solid support to react with sulfhydryl reagents, was employed to determine aging of silica/APTES/PMPI supports as a function of PMPI to APTES coverage. FIG. 67 shows the percentage of active maleimides remaining on a support when stored under pH 7 buffer for up to one week. The initial coverage of APTES residues and PMPI residues, determined from transmission infrared spectra using equations (6.1) and (6.2), are indicated in the legend. As noted earlier, an excess of immobilized PMPI residues over those of APTES is typically observed. This is attributed to PMPI attachment in the shape of multimer adducts as discussed in context of FIG. 66.

When the APTES monolayer amines are fully consumed by reaction with PMPI (filled circles, FIG. 67), the decrease in activity of the support parallels that for maleimides in solution (stars, FIG. 67) with about 50% activity remaining after 24 h of storage under pH 7 phosphate buffer. It is noted that, for bulk solution studies, the water-soluble bismaleimide BM(PEO)₄ was used in lieu of PMPI to avoid the aforementioned generation of aromatic amines on PMPI whose ability to react with maleimides would compete with that of Ellman's reagent. In contrast to the fully reacted support, when APTES amines are only partially consumed by PMPI (open circles, FIG. 67), leaving unreacted amine groups on the surface, there is a fast initial decrease in maleimide activity. This drop is attributed to deactivation of PMPI maleimides through reaction with the remnant amines of the solid support.

The maleimide activity of silica/APTES/PMPI supports was also investigated when supports were stored in air. After one week of air storage in a sealed vial, 60% of maleimides were still active. Air storage is therefore recommended if activity of the support must be preserved for periods of days prior to modification with nucleic acids or other molecules. Precautions may be taken to also minimize adsorption of atmospheric contaminants if surfaces stored for long periods are to behave reproducibly.

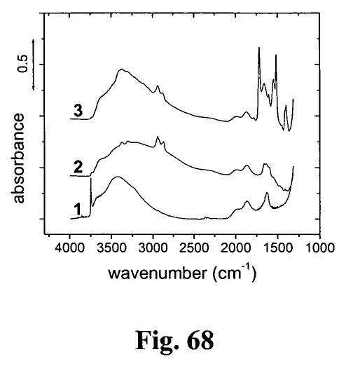

Infrared spectroscopy was used to confirm and further detail the chemical changes taking place as silica/APTES/PMPI supports age. FIG. 68 depicts mid-IR spectra of unmodified silica, after modification with APTES, and after subsequent modification with PMPI. These spectra serve as reference for subsequent discussion as they were taken immediately after sample preparation, so that changes associated with aging of the support were minimal. Assignments of the principal spectral bands have been reported previously.

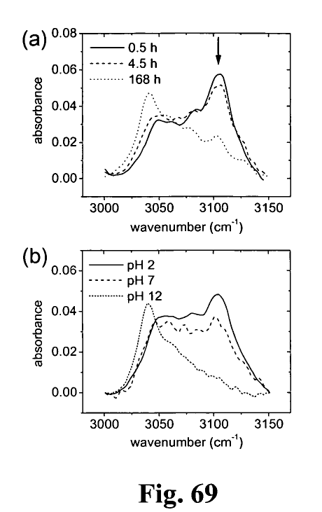

In FIG. 69, close-ups of the aromatic and maleimide C—H stretching region are shown for samples immersed for different times (FIG. 69a) and in different pH (FIG. 69b) buffers. In samples with an intact maleimide olefinic bond, a C—H stretch is observed close to 3100 cm⁻¹. With time, this band is reduced in intensity. A decrease is already apparent after just 4.5 hours of storage under pH 7 buffer, and after 7 days this band has largely disappeared (FIG. 69a). The IR data corroborate the conclusion that a gradual degradation of maleimide activity occurs under neutral conditions. The mechanism of degradation is believed to take place by addition of ^{—}OH to the imide double bond, —COCH═CHCO-→-COCH(OH)CH₂CO—. Such a mechanism is also consistent with the observed accelerated disappearance of the maleimide C—H stretch under alkaline conditions (FIG. 69b).

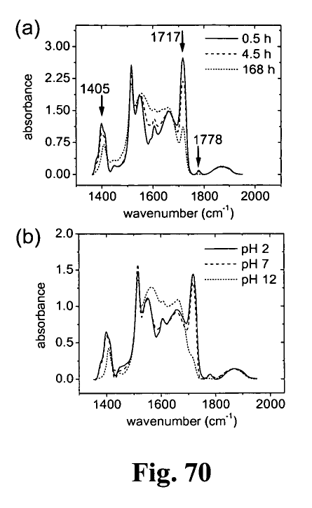

Spectral changes in the 1350 to 1800 cm⁻¹ region, where multiple PMPI bands appear, provide additional information on chemical transformations taking place on the silica/APTES/PMPI supports. Several bands, most notably the symmetric (1778 cm⁻¹) and asymmetric (1717 cm⁻¹) maleimide C═O stretches, and the maleimide symmetric C—N—C stretch (˜1405 cm⁻¹), decrease as a function of storage time (FIG. 70a) and at elevated pH (FIG. 70b). The attrition of these bands is consistent with opening of the maleimide ring and, as seen from FIG. 70b, is facilitated by increased buffer basicity. N-alkyl maleimides are known to undergo hydrolysis under basic conditions (Matsui) as a consequence of attack of ^{—}OH on a carbonyl carbon of the imide 1, severing the carbon-nitrogen bond and producing 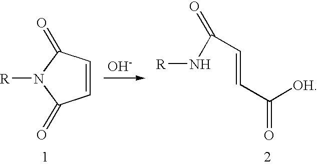
the N-alkylmaleamic acid 2

In addition to suppression of the above-mentioned bands, imide hydrolysis should lead to enhanced amide I and II modes (˜1660 and ˜1560 cm⁻¹, respectively), to carboxylic acid C═O stretching between 1650 and 1680 cm⁻¹ (if hydrogen bonded), and to bands from acid salts —COO⁻ between 1550 to 1650 cm⁻¹ (asymmetric stretch) and between 1330 and 1440 cm⁻¹ (symmetric stretch, broad band). These trends are consistent with the generally increased absorbance seen in these regions, though complexity of the spectra and band overlap prevent detailed assignments.

In the related reaction between N-phenyl maleimide (NPM) and APTES-modified silica, a slow transamidation involving APTES amines and maleimide carbonyl carbons was noted. The transamidation opened up the NPM imide ring, causing spectral changes similar to those associated with hydrolysis. In the case of PMPI-modified supports, this transamidation mechanism is not believed to be significant since few if any APTES amines remain available after immobilization of PMPI. The virtually complete consumption of amines during assembly of the PMPI layer is seen in the absence of the NH₂ stretch doublet at 3300-3400 cm⁻¹ for silica/APTES/PMPI supports (compare curves 2 and 3 in FIG. 68).

In summary of this section, titration and spectroscopic analyses indicate that maleimide-derivatized silica supports progressively lose activity toward sulfhydryls over days when stored under pH 7 buffer. Activity may be retained for longer periods by storage under air. Under buffer, deactivation is accelerated under alkaline conditions, presumed to proceed by reaction of the imide C═C bond with hydroxide ions. Concomitantly, opening of the imide ring occurs by hydrolysis at carbonyl sites. Should both carbonyl sites undergo hydrolysis, any biomolecules immobilized via the imide end would be removed from the support (FIG. 66). Similar outcome would result should hydrolysis of the APTES-PMPI urea linkage occur. In either hydrolysis scenario, primary amines would be generated on the solid support. It is therefore significant to note that amine N—H stretch bands were not observed in silica/APTES/PMPI IR spectra even after 7 days under pH 7 buffer. This suggests that hydrolysis was not so extensive as to fully cleave majority of the imide rings or urea bonds. Further discussion of stability of DNA attachment on these supports is presented in the following section.

Probe Attachment. Single-stranded, thiol-terminated DNA oligonucleotides P1 were attached to APTES and PMPI modified glass slides. Powder silica supports were not used on account of their prohibitively high surface areas to consider for DNA modification. Modified slides were investigated with XPS.

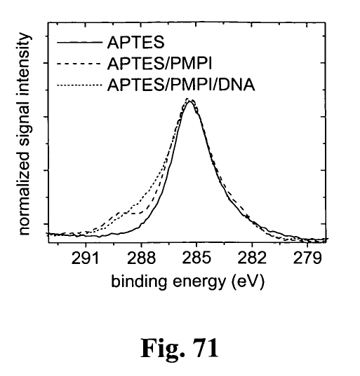

FIG. 71 shows normalized XPS C 1s traces from APTES, APTES/PMPI, and APTES/PMPI/DNA surfaces. Addition of PMPI residues is signified by appearance of a a rather prominent shoulder at higher binding energy, attributed to carbonyl carbons (FIG. 66). After DNA attachment, the C 1s trace exhibits an overall broadening on account of diversity of bonding configurations found in DNA carbons (refs).

The areal coverage of APTES and PMPI residues was estimated from N 1s intensities, using equation (6.3) with X_{N1s}=1948 barns, θ=45°, and independently measured instrument response function R as described in Materials and Methods. For APTES films, a coverage of 2.5 nm⁻² was determined from the total N 1s intensity after correction for background N 1s signal measured from clean slides. This coverage is an overestimate as it is reported per geometric area. Presence of surface roughness increases the effective area seen by the XPS analyzer. Based on electrochemical measurements of surface roughness from gold-coated glass slides of same batch, we do not expect roughness to increase the surface area by more than 30%. Therefore, the true APTES coverage is estimated to lie within 1.9 and 2.5 residues/nm².

PMPI coverage was calculated from the increase in total N 1s signal following reaction with APTES, yielding 1.8 nm⁻² PMPI residues. In addition to roughness corrections, PMPI coverage is subject to signal attenuation as, relative to an APTES film, nitrogen atoms will be more buried and hence their emission more affected by passage through the organic layer. The PMPI coverage will therefore be somewhat greater than the 1.8 nm⁻² derived from attenuated signals. Regardless, maleimide coverage is estimated to be close to 2 nm⁻², more than 10 times the typical DNA coverage (see below) of 0.1 nm⁻² or less. The excess of maleimide groups raises prospects that a DNA chain, having attached through a sulfhydryl terminus as in FIG. 66, may further react with nearby maleimides. Even mildly nucleophilic moieties, such as aromatic base amines, could potentially react if allowed sufficient time. Additional crosslinking would lead to multipoint attachment that could strongly interfere with hybridization activity of immobilized strands.

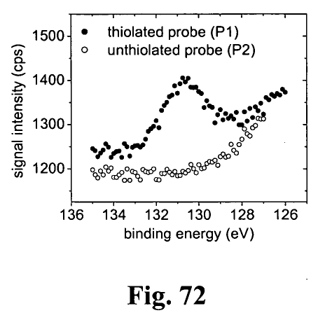

Attachment of strands other than through the sulfhydryl terminus was tested for by comparison of immobilized coverages of P1 and P2 oligonucleotides. The P2 sequence lacks the terminal sulfhydryl but is otherwise identical to P1. FIG. 72 depicts raw P 2p traces measured from maleimide-functional slides after immersion in oligonucleotide solutions for 5 days. Presence of P 2p emission is indicative of DNA attachment as no other source of phosphorus atoms was used during sample preparation. After 5 days, maleimide activity will have largely decayed due to hydrolysis (FIG. 2) so that potential for further reactions between the surface and oligonucleotides is minimal. From FIG. 72, within sensitivity limits of the XPS data (˜5×10¹¹ chains/cm²), P2 chains did not attach. In contrast, a surface coverage of 1.9×10¹² chains/cm² is found for the P1 oligonucleotides (via equation 6.1 and 6.2 as described in Materials and Methods). Same conclusions were reached in experiments in which shorter contact times were used, including 2 h, 5 h, and 2 days. As in the 5-day experiments, for these shorter times P 2p emissions were only detected from surfaces exposed to the thiolated P1 strands. These data indicate that attachment of sulfhydryl-terminated oligonucleotides to maleimide-functional supports is highly site-specific, and that virtually all strands immobilized by one end only.

### REFERENCES FOR EXAMPLE 6

[1]. A B Steel, T M Herne and M J Tarlov (1998). Electrochemical quantitation of DNA immobilized on gold. Anal. Chem. 70: 4670-4677.
[2]. T M Herne and M J Tarlov (1997). Characterization of DNA Probes Immobilized on Gold Surfaces. J. Am. Chem. Soc. 119: 8916-8920.
[3]. R Levicky, T M Herne, M J Tarlov and S K Satija (1998). Using self-assembly to control the structure of DNA monolayers on gold: A neutron reflectivity study. J. Am. Chem. Soc. 120: 9787-9792.
[4]. A W Peterson, R J Heaton and R Georgiadis (2000). Kinetic control of hybridization in surface immobilized DNA monolayer films. J. Am. Chem. Soc. 122: 7837-7838.
[5]. A W Peterson, R J Heaton and R M Georgiadis (2001). The effect of surface probe density on DNA hybridization. Nucl. Acids Res. 29: 5163-5168.
[6]. R M Corn (2004). Personnal Communication.
[7]. J H Scofield (1976). Hartree-Slater subshell photoionization cross-sections at 1254 and 1487 eV. J. Electron. Spectrosc. 8: 129-137.
[8]. C J Powell and A Jablonski (2001). NIST Electron Effective-Attenuation-Length Database—Version 1.0. National Institute of Standards and Technology: Gaithersburg, Md.

### EXAMPLE 7

### Reaction of N-Phenyl Maleimide with Aminosilane Monolayers

Introduction. Attachment of biomolecules to surfaces underpins a number of separation and diagnostic technologies [1]. On siliceous surfaces attachment is typically mediated by first silylating the surface followed by immobilization of the biomolecule of interest. Aminosilanes such as (aminopropyl)triethoxysilane (APTES) have featured prominently for such applications, due in part to significant advances in understanding this class of surface modification agents [2]. Notably, aminosilanes have the advantage of catalytic activity by the amine group that facilitates formation of siloxane bonds with surface silanols [2-4], mitigating and potentially obviating the need for post-depositional curing.

This Example investigates reaction of N-phenyl maleimide (NPM) with monolayers of APTES formed on amorphous silica. Maleimide moieties are commonly introduced to biological molecules for a variety of crosslinking or conjugation applications, including immobilization to glass and other types of solid supports [5-14]. Usually, the desired reaction is addition of a thiol (—SH) group to the maleimide C═C double bond to form a sulfide linkage. A recent report indicated that reaction of APTES-modified surfaces with maleimide groups also proceeds readily, presumably by addition of the amine to the olefinic double bond of the maleimide [15]. amines possess certain advantages over thiols as they are not susceptible to oxidation and formation of disulfides. For example, bismaleimide compounds could be used as crosslinkers between aminosilanized surfaces and amino-modified biomolecules, without the need for protected thiol groups which typically involve reduction and purification steps prior to surface immobilization [7, 15, 16]. More facile and direct immobilization methods are of interest as they simplify protocols with concomitant improvements in reproducibility and cost effectiveness.

In this Example, mechanism of attachment between APTES monolayers and NPM is investigated as a model for interfacial amine-maleimide reactions. Infrared spectroscopy serves as a main investigative tool, supported by titration and elemental analyses. Attachment proceeds both by Michael addition to the unsaturated olefinic bond of the maleimide as well as by transamidation at the maleimide carbonyl groups. Moreover, stability of resultant APTES-NPM monolayers is investigated with view to identifying avenues for further improvement. Thus, stability is governed by APTES leaching from the silica rather than degradation of NPM-APTES residues.

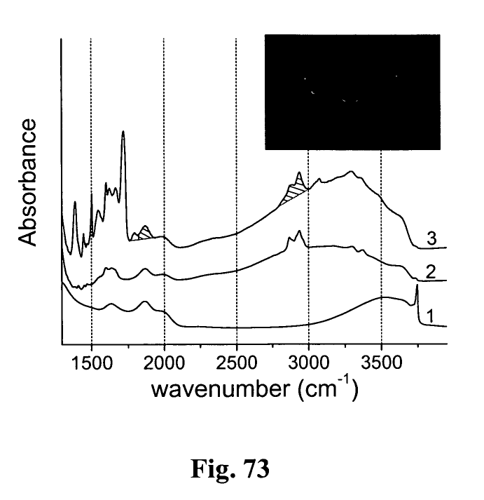

Materials and Methods. Aerosil 200 fumed silica was donated by Degussa-Hüls. This free-flowing powder was measured to possess a Brunauer-Emmett-Teller (BET) specific surface area of 200 m²/g (Micromeritics Instrument Corp.). Its high surface area makes fumed silica well suited to the present study, as chemical modifications of the surface is readily monitored with standard analytical methods such as transmission infrared spectroscopy and titration analysis. Structurally, the silica consists of 12 nm diameter solid particles, with a purity of 99.8% amorphous SiO₂. The primary particles aggregate into larger grains (FIG. 73 inset). (Aminopropyl)triethoxysilane (APTES, 98%), N-phenylmaleimide (NPM, 97%), L-cysteine (98%), and anhydrous toluene and acetonitrile were purchased from Aldrich and used as received. Ellman's reagent (DTNB; 5,5′-dithio-bis(2-nitrobenzoic acid)) and p-maleimidophenyl isocyanate (PMPI) were from Pierce Biotechnology. Potassium phosphate (99.4%) and sodium chloride (100.0%) were obtained from Fisher Scientific. Purified water (18.4 MΩ cm), provided by a Millipore water purification system, was used in preparation of phosphate buffers (PBS: 10 mM potassium phosphate, 0.1 M NaCl, pH 7).

Surface Modification. Fumed silica was silanized with APTES as previously described [15]. In brief, silica powder was immersed in 1% w/w APTES in anhydrous toluene for 30 minutes with agitation. The powder was centrifuged and the supernatant removed, followed by a sequence of two washes with anhydrous toluene, one wash with deionized water, and a last wash with acetonitrile. For each wash, the powder was mechanically redispersed in the solvent and then re-centrifuged, followed by removal of the supernatant. The washed powder was dried overnight at 100° C. As noted earlier, this attachment protocol results in partial monolayers of APTES with a coverage of about 1 residue/nm [15], as compared to coverages of about 2 residues/nm reported in most literature studies of APTES monolayers prepared under anhydrous conditions [17-20]. The lower coverages are chiefly attributed to washing the silica with deionized water prior to curing, which is expected to remove silane molecules that are not already covalently bonded prior to curing.

APTES-derivatized powders were reacted with solutions of NPM or PMPI in anhydrous acetonitrile at a typical concentration of 40 mM, corresponding to 3 molecules charged per each nm² of surface area present. Reaction was allowed to proceed for 2 h at room temperature, after which the powders were washed five times with anhydrous acetonitrile as described above, followed by overnight drying at 50° C. The dried powders were characterized with infrared spectroscopy, elemental analysis, and titration analysis.

Characterization Methods. Elemental analysis for carbon, nitrogen, and hydrogen content was performed by Galbraith Laboratories. NPM coverages were calculated from the increase in mass percent of nitrogen in APTES-NPM powders over that in precursor, APTES-only powder. For comparison, NPM coverages were also calculated based on carbon content. These values systematically exceeded those based on nitrogen by 5 to 10%. The difference is attributed to adventitious carbonaceous materials introduced during handling, shipping, and analysis of the samples.

Fourier transform infrared (FTIR) spectroscopy was performed in transmission on approximately 1 mg of modified powder sandwiched in a chamber created by a circular gasket and two CaCl₂ windows. Each spectrum was an average of 1000 scans collected at 2 cm⁻¹ resolution on a Nicolet Magna 560 IR spectrometer equipped with a mid-IR, liquid nitrogen cooled MCT detector. Care was taken to ensure uniform distribution of powder within the sample chamber, and to minimize scattering losses. Background scans were collected similarly but using a clear beam path.

APTES coverages were estimated from FTIR spectra using the calibration relation:

$$APTES \text{/} {nm}^{2} = \frac{1}{1.89} ( \frac{{\int }_{2800}^{3000} A ( v ) ⅆ v}{{\int }_{1820}^{ 1920} A ( v ) ⅆ v} ) ( {r}^{2} = 0.994 ) \qquad ( 7.1 )$$

In equation (7.1), A(v) is absorbance measured at wavenumber v. The ratio of the 2800-3000 cm⁻¹ (APTES C—H stretches) to 1820-1920 cm⁻¹ (silica structural overtone vibrations [21, 22]) integrals is proportional to APTES coverage per area of surface in the IR beam. The calibration was recalculated with tighter integration limits than in prior work [15] to minimize interference from symmetric C═O stretching of the succinimide ring formed upon reaction of NPM with APTES. Prior to performing the integrals, a baseline correction was applied as indicated in FIG. 73. Equation (7.1) is based on calibration of the infrared absorbances with absolute APTES coverages as determined by elemental analysis.

The presence of α,β unsaturation in immobilized NPM residues was quantified using Ellman's assay [23, 24], following provider instructions (Pierce Biotechnology). L-cysteine was used as the titrant for 2 mg aliquots of functionalized powder. Decrease in bulk concentration of L-cysteine, due to addition to olefinic double bonds of NPM residues on modified powders, was monitored spectrophotometrically with DTNB. DTNB reacts in a disulfide-thiol exchange reaction with available L-cysteine thiols to yield a mixed disulfide and the colored species 2-nitro-5-thiobenzoic acid, which is detected spectrophotometrically and compared against a calibration curve. Surface coverage of α,β unsaturation was estimated from measured decreases in L-cysteine, assuming 1:1 stoichiometry of reaction with maleimide C═C bonds. APTES modified silica served as control.

Results and Discussion. FIG. 73 shows characteristic mid-IR spectra of silica powder before modification, after modification with APTES, and after further reaction with NPM. Table 7.1 lists assignments of the principal spectral features. Notable changes associated with APTES modification include disappearance of free silanol O—H stretch at 3744 cm⁻¹, consistent with formation of APTES-silica siloxane bonds, and appearance of primary amine stretches at 3303 and 3370 cm⁻¹, C—H stretch bands in the 2800-3000 cm⁻¹ region and, less prominently, NH₂ bend at 1600 cm⁻¹, CH₂ bend at 1470 cm⁻¹, and Si—CH₂ bend at 1412 cm⁻¹. Spectral features arising from subsequent powder modification with NPM are discussed below.

TABLE 7.1
Mid-IR Spectral Assignments

| | |
|---|---|
| Mode (cm⁻¹)^{a} | Attribution |

neat silica (references [21, 22])

| | |
|---|---|
| 3745 s | free silanol O—H stretching |
| 3760-3300 | total silanol hydroxyls band, adsorbed H₂O |
| 1980 sh, 1870 m | overtone structure vibrations of SiO₂ lattice |
| 1630 m | SiO₂ lattice vibrations; bending O—H (molecular |
| | water) |

APTES residues on silica (reference [31])

| | |
|---|---|
| 3370 w | N—H asymmetric stretch |
| 3303 w | N—H symmetric stretch |
| 2937 m | CH₂ asymmetric stretch |
| 2869 m | CH₂ symmetric stretch |
| 1600 w | N—H deformation |
| 1469 w | CH₂ deformation |

NPM residues reacted with APTES-silica

| | |
|---|---|
| 3100-3000 w | aromatic C—H stretch (ref [30]) |
| 1793 w | succinimide symmetric C═O stretch (ref [32]) |
| 1718 s | succinimide asymmetric C═O stretch (ref [32]) |
| 1664 m | amide I (ref [33]) |
| 1600 m | aromatic C═C stretch (ref [30]) |
| 1547 m | amide II (ref [33]) |
| 1502 s | aromatic C═C stretch (ref [30]) |
| 1446 m | N—H deformation, cis-amide (ref [33]) |
| 1390 s | succinimide symmetric C—N—C stretch (tentative, |
| | ref [30]) |

^{a}s = strong; m = medium; w = weak

Elemental analysis showed that addition of NPM to surface amines was not stoichiometric; rather, NPM coverage was consistently less than that of APTES. Reaction of silica bearing 1.15 APTES residues/nm² with sufficient NPM to supply 1.3 molecules per nm² of powder surface (acetonitrile, 2 h) yielded an NPM coverage of 0.73 residues/nm². Doubling the NPM concentration increased surface loading only slightly, to 0.86 residues/nm², corresponding to 75% conversion of APTES amines. Therefore, even in presence of excess NPM reagent, addition of NPM to APTES residues did not proceed to completion within the 2 h period.

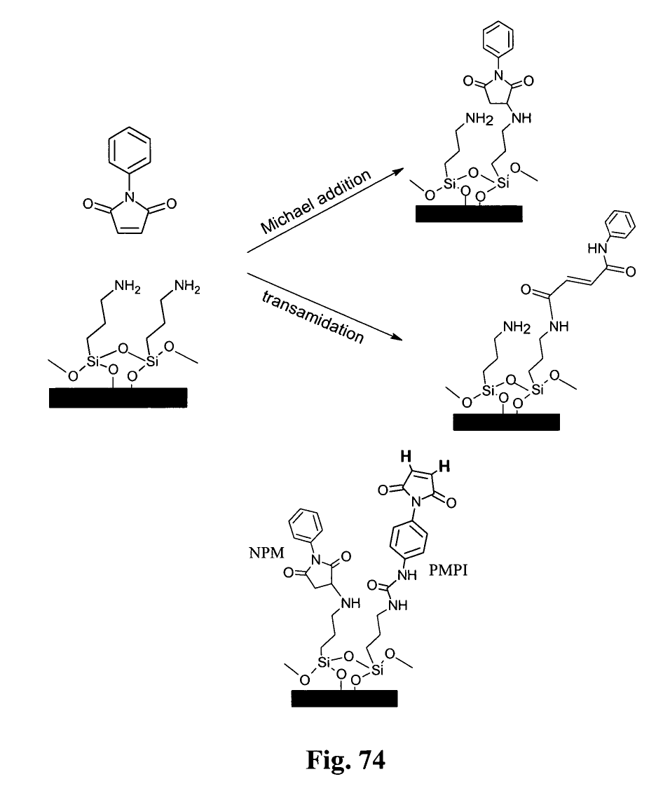

Prospective reactions of NPM with APTES amines include Michael addition to the maleimide alkene bond and transamidation at the carbonyl C atoms. These mechanisms are illustrated in FIG. 74. Michael addition of amines to maleimides is used widely in polymerizations [25-27], typically carried out in the melt or in organic solvents. In aqueous solutions, Michael addition has been used to conjugate amino groups of peptides to maleimides [28]. Smyth et al also reported occurrence of intramolecular transamidation involving the α-amino group of a cysteine residue and imide carbonyls, the cysteine having been previously conjugated to the maleimide via thioether linkage at the imide olefinic double bond [29].

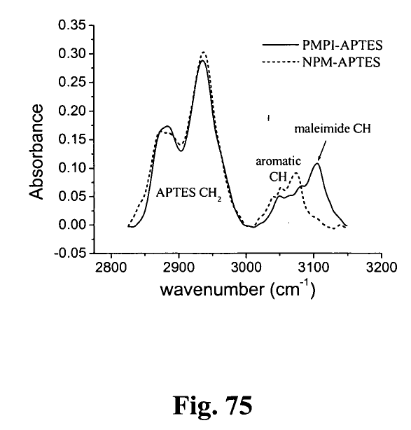

The attachment mechanism between NPM and APTES monolayers was investigated using IR spectroscopy and Ellman's titration analysis as described in the experimental section. NPM-modified powders were prepared using an excess of 3 NPM molecules per nm² of surface, corresponding to concentration of about 40 mM NPM in acetonitrile. Titration analysis revealed negligible remnant alkene unsaturation, with an average over five samples yielding 0.02±0.02 alkene bonds/nm². Since these samples had been dried overnight, measurements were also performed on wet (with acetonitrile) powders to determine remnant unsaturation immediately after NPM attachment. The wet samples yielded 0.012±0.005 alkene bonds/nm². Thus both dried and wet powders exhibited very low activities of α,β unsaturation, supporting Michael addition as the predominant reaction mechanism. This conclusion is further supported by lack of maleimide C—H stretching at 3107 cm^{-1 [}30] in IR spectra. In contrast, this band was present in spectra of APTES powders modified with p-maleimidophenyl isocyanate (PMPI), as shown in FIG. 75. The highly reactive isocyanate group of PMPI reacts with APTES in strong preference to its maleimide terminus [15], thus preserving the maleimide C═C double bond.

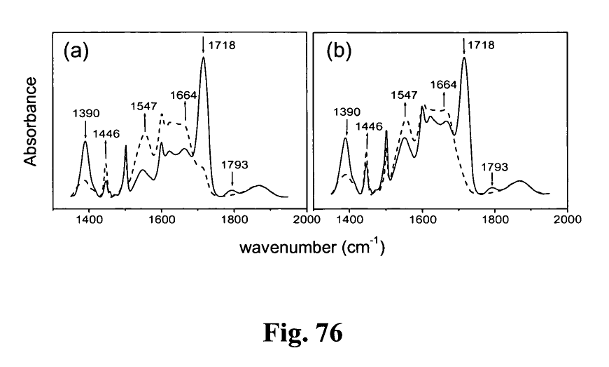

Exclusive Michael addition cannot, however, explain other observed spectral features. In particular, amide I and II absorptions at 1665 cm⁻¹ and 1546 cm⁻¹, respectively, and a line attributed to cis-amide N—H bending at 1446 cm⁻¹, were inadvertently present. As displayed in FIG. 76a, these lines increased in intensity upon storage of modified powders in PBS at room temperature. The likely explanation for these trends is progression of slow transamidation between APTES amines and imide carbonyl sites, with amide bonds continuing to form on the time scale of hours following initial attachment via Michael addition. For a similar scenario of an intramolecular transamidation reaction between an amine and an imide carbonyl group, Smyth et al reported nearly complete conversion after 36 h under mildly alkaline conditions [29]. These long time scales are comparable to those observed in the present study. Occurrence of transamidation is also in line with diminishing symmetrical and asymmetrical succinimide carbonyl stretches at 1793 cm⁻¹ and 1718 cm⁻¹ (FIG. 76a), reflecting opening of the imide ring. Such residues, having undergone initial Michael addition followed by transamidation, will be left with a bidentate attachment to the surface. Significantly, after 24 h of storage under PBS, the —N—H stretch doublet (not shown) became indistinguishable, signifying near complete absence of primary amines. This observation is attributed to their incorporation into amide bonds with NPM residues. The band at 1390 cm⁻¹ also decreased markedly with time (FIG. 76a); based on assignments of Parker et al for NPM [30] a speculative assignment of this band is symmetrical C—N—C stretching of phenylsuccinimide. As the data of FIG. 76b show, elevated temperatures accelerated the transamidation reaction.

Hydrolysis of the imide ring is an alternate possibility for the decrease in carbonyl imide bands. Hydrolyzed products should exhibit carboxylic acid lines, which would likely include hydrogen bonded species as well as acid salts. Such features could not be identified in the spectra; however, such identification was expected to be difficult due to overlap with other bands. Nevertheless, stability (see below) of APTES-NPM conjugation argues that hydrolysis, if present, was not extensive. Hydrolysis at both carbonyl sites of an imide ring would cleave the NPM phenyl ring from the support, what should result in a decreased ratio of phenyl to APTES bands. As discussed below such a decrease was not observed.

In summary, combination of Ellman's titration and spectral evidence indicates that NPM molecules first add to APTES monolayers predominantly via Michael addition, continued by a slower second stage in which additional cross-linking occurs by transamidation. The facile reaction at the imide olefinic bond agrees with results of Gambogi and Blum [27], who used NMR to study local dynamics of aminosilanized silica surfaces in contact with a bismaleimide resin. These authors confirmed that ready reaction of amines with the unsaturated imide C═C bond takes place. The possibility of slower, subsequent transamidation was not addressed.

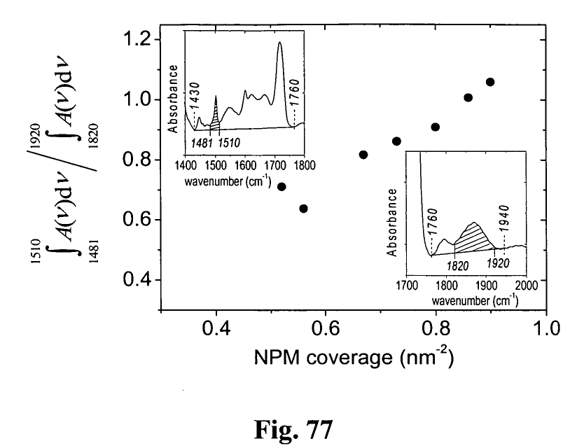

Stability of attachment is an important concern for exploiting maleimide-amine coupling for immobilization of biomolecules. As a preliminary assessment, two series of experiments were carried out. In the first series, changes in relative NPM and APTES coverage were monitored under storage in PBS buffer at room temperature. In the second, samples were immersed in heated PBS buffer at temperatures ranging from 30° C. to 90° C. for 2 h. A relative measure of NPM to APTES coverage was calculated by dividing the integrated intensity of the aromatic band at 1500 cm⁻¹, which reasonably correlated with amount of NPM as verified by cross-comparison with elemental analysis (FIG. 77), by the integrated intensity of the APTES C—H stretches. Other prominent NPM bands (e.g. associated with imide or amide modes) were unsuitable on account of intensity changes due to aforementioned chemical transformations. Absolute coverage of APTES was calculated from intensity of alkyl C—H bands via equation (7.1).

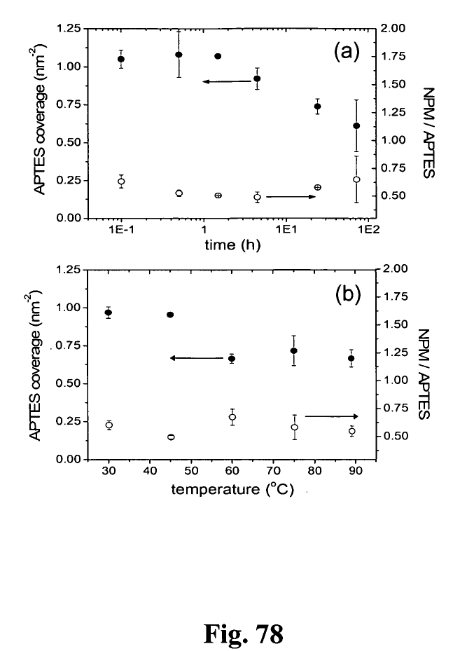

FIG. 78a plots changes in APTES and relative NPM coverage as a function of storage under PBS buffer. After three days, approximately a third of APTES residues was cleaved from the surface. Over this time, the ratio of NPM to APTES remained constant within accuracy of measurement. Similar conclusions are reached from thermal stability analysis in FIG. 78b, where it is again observed that the ratio of NPM to APTES remained unchanged. The constancy of the coverage ratio suggests that NPM is lost at a rate determined by APTES; in other words, that the weak link in the attachment is lability of siloxane bonds between APTES and silica rather than cleavage at some internal position, such as amide groups, within APTES-NPM adducts.

Conclusion. Reaction of N-phenyl maleimide (NPM) with aminosilane APTES monolayers, prepared on fumed silica supports, proceeds readily in anhydrous acetonitrile under ambient conditions by addition of silane amines to unsaturated C═C bonds of the imides. Nearly all NPM molecules attached initially via this mechanism. However, upon storage under neutral buffer, a secondary process was also detected in which surface amines reacted at the carbonyl sites of immobilized NPM residues to produce amide linkages. Therefore, structure of the NPM layer evolved after initial attachment to yield a distribution of products on the surface, with a fraction of NPM residues reacting through both alkene and carbonyl sites. Stability of thus modified surfaces under neutral buffer at up to 90° C. was investigated. A gradual loss of NPM and APTES residues from the silica support was observed, with the loss of NPM paralleling that of APTES. This indicated that stability was governed by cleavage of APTES residues from the surface. Improvements to robustness will require enhancing stability of the silane layer, for example, by forming fuller layers in which silane-silane cross-linking is more extensive compared to the present films, in which APTES coverage was only about half a full monolayer.

### REFERENCES TO EXAMPLE 7

[1] T. Cass, F. S. Ligler (Eds.), Immobilized Biomolecules in Analysis, Oxford University Press Inc., New York, 1998.
[2] E. F. Vansant, P. Van Der Voort, K. C. Vrancken, Characterization and Chemical Modification of the Silica Surface, Elsevier, New York, 1995, Chapter 9.
[3] J. Blitz, R. S. S. Murthy, D. E. Leyden, J. Am. Chem. Soc. 109 (1987) 7141.
[4] L. D. White, C. P. Tripp, J. Colloid Interface Sci. 227 (2000) 237.
[5] M. Brinkley, Bioconjugate Chem. 3 (1992) 2.
[6] E. Ishikawa, M. Imagawa, S. Hashida, S. Yoshitake, Y. Hamaguchi, T. Ueno, J. Immunoassay 4 (1983) 209.
[7] L. A. Chrisey, G. U. Lee, C. E. O'Ferrall, Nucl. Acids Res. 24 (1996) 3031.
[8] C. Adessi, G. Matton, G. Ayala, G. Turcatti, J.-J. Mermod, P. Mayer, E. Kawashima, Nucl. Acids Res. 28 (2000) e87.
[9] T. Okamoto, T. Suzuki, N. Yamamoto, Nat. Biotechnol. 18 (2000) 438.
[10] S. J. Oh, S. J. Cho, C. O. Kim, J. W. Park, Langmuir 18 (2002) 1764.
[11] S.-J. Xiao, M. Textor, N. D. Spencer, Langmuir 14 (1998) 5507.
[12] G. MacBeath, A. N. Koehler, S. L. Schreiber, J. Am. Chem. Soc. 121 (1999) 7967.
[13] H. G. Hong, P. W. Bohn, S. G. Sligar, Anal. Chem. 65 (1993) 1635.
[14] P. A. Johnson, R. Levicky, Langmuir 19 (2003) 10288.
[15] L. Jin, A. Horgan, R. Levicky, Langmuir 19 (2003) 6968.
[16] M. J. O'Donnell, K. Tang, H. Koster, C. L. Smith, C. R. Cantor, Anal. Chem. 69 (1997) 2438.
[17] G. S. Caravajal, D. E. Leyden, G. R. Quinting, G. E. Maciel, Anal. Chem. 60 (1988) 1776.
[18] K. M. R. Kallury, P. M. Macdonald, M. Thompson, Langmuir 10 (1994) 492.
[19] P. Trens, R. Denoyel, Langmuir 12 (1996) 2781.
[20] K. C. Vrancken, P. Van Der Voort, K. Possemiers, E. F. Vansant, J. Colloid Interface Sci. 174 (1995) 86.
[21] E. F. Vansant, P. Van Der Voort, K. C. Vrancken, Characterization and Chemical Modification of the Silica Surface, Elsevier, New York, 1995, p. 65.
[22] D. Gorski, E. Klemm, P. Fink, H.-H. Horhold, J. Coll. Int. Sci. 126 (1988) 445.
[23] G. L. Ellman, Arch. Biochem. Biophys. 82 (1959) 70.
[24] P. W. Riddles, R. L. Blakeley, B. Zemer, Anal. Biochem. 94 (1979) 75.
[25] J. V. Crivello, J. Polym. Sci. Pol. Chem. 11 (1973) 1185.
[26] Z. Shen, J. R. Schlup, J. Appl. Pol. Sci. 67 (1998) 267.
[27] J. E. Gambogi, F. D. Blum, Macromolecules 25 (1992) 4526.
[28] D. G. Smyth, O. O. Blumenfeld, W. Konigsberg, Biochem. J. 91 (1964) 589.
[29] D. G. Smyth, A. Nagamatsu, J. S. Fruton, 82 (1960) 4600.
[30] S. F. Parker, S. M. Mason, K. P. J. Williams, Spectrochim. Acta 46A (1990) 315.
[31] C. H. Chiang, H. Ishida, J. L. Koenig, J. Colloid Interface Sci. 74 (1980) 396.
[32] A. Pistorius, P. J. T. A. Groenen, W. J. Degrip, Int. J. Peptide Protein Res. 42 (1993) 570.
[33] G. Socrates, Infrared Characteristic Group Frequencies, John Wiley & Sons Inc., New York, 1994.

What is claimed:

1. A method for immobilizing molecules on a siliceous surface comprising:
(a) silylating a siliceous substrate wherein the substrate comprises a silanol surface, with an aminosilane thereby forming a modified siliceous substrate, wherein the modified siliceous substrate comprises an aminosilanized surface;
(b) reacting the aminosilanized surface with a heterobifunctional crosslinker, thereby forming a further modified siliceous substrate comprising a maleimide surface;
(c) reacting the maleimide surface with thiolated molecules, wherein the thiolated molecules comprise thiol groups, and wherein the reaction between the maleimide surface and the thiol groups form thioether linkages, thereby forming a siliceous surface comprising immobilized molecules.

2. The method of claim 1, wherein the siliceous substrate comprises amorphous silica, fumed amorphous silica, fused silica, or a combination thereof.

3. The method of claim 1, wherein the aminosilane comprises aminopropyl)dimethylethoxysilane (APDMES); (3-aminopropyl)triethoxysilane (APTES); alkyl trichlorosilane, tetramethoxysilane, tetraethoxysilane, tetrapropoxysilane, methyltrimethoxysilane, methyltriethoxysilane, methyltris(methylethylketoxime)silane, methyltris(acetoxime)silane, methyltris(methylisobutylketoxime)silane, dimethyldi(methylethylketoxime)silane, trimethyl(methylethylketoxime)silane, vinyltris(methylethylketoxime)silane, methylvinyldi(methylethylketoxime)silane, methylvinyldi(cyclohexanoneoxime)silane, vinyltris(methylisobutylketoxime)silane, phenyltris(methylethylketoxime)silane, methyltriacetoxysilane, or tetracetoxysilane substituted with an amine group; or a combination thereof.

4. The method of claim 3, wherein the aminosilane comprises APDMES, APTES, or a combination thereof.

5. The method of claim 4, wherein the aminosilane comprises APTES.

6. The method of claim 1, wherein the heterobifunctional crosslinker comprises N-(p-maleimidophenyl)isocyanate (PMPI), m-maleimidobenzoyl-N-hydroxysuccinimide ester (MBS) or N-(γ-maleimidobutyryloxy)sulfosuccinimide ester (sulfo-GMBS).

7. The method of claim 1, wherein the thiolated molecules comprise thiolated-nucleic acids, thiolated-peptides, thiolated-lipids, thiolated-sugars or any combination thereof.

8. The method of claim 1, wherein the heterobifunctional crosslinker comprises an isocyanate group and a maleimide group.

9. The method of claim 1, wherein the aminosilane comprises APTES, wherein the heterobifunctional crosslinker comprises PMPI, and wherein the biomolecules comprise thiolated-nucleic acids.

10. A method for immobilizing molecules on a metal-film surface comprising:
(a) attaching a thiol-derivatized polysiloxane on a metal-film substrate thereby forming a modified metal-film substrate comprising a thiol surface;
(b) reacting the thiol surface with a bismaleimide crosslinker, thereby forming a further modified metal-film substrate comprising a maleimide surface; and
(c) reacting the maleimide surface with thiolated molecules, wherein the thiolated molecules comprise thiol groups, and wherein the reaction between the maleimide surface and the thiol groups form thioether linkages, thereby forming a metal-film surface comprising immobilized molecules.

11. A method for immobilizing molecules on a metal-film surface comprising:
(a) attaching a thiol-derivatized polysiloxane on a metal-film substrate thereby forming a modified metal-substrate comprising thiol groups; and
(b) reacting the modified metal-substrate with maleimide-modified molecules, wherein the maleimide-modified molecules comprise maleimide groups, and wherein the reaction between the thiol groups of the modified metal-substrate and the maleimide groups form thioether linkages, thereby forming a metal-film surface comprising immobilized molecules.

12. The method of any of claims 10 or 11, wherein the thiol-derivatized polysiloxane is poly(mercaptopropyl)methylsiloxane (PMPMS).

13. The method of any one of claims 10 or 11, wherein the metal-film substrate comprises cadmium, chromium, cobalt, copper, gold, hafnium, iridium, iron, manganese, mercury, molybdenum, nickel, niobium (columbium), osmium, palladium, platinum, rhenium, rhodium, ruthenium, scandium, silver, tantalum, technetium, titanium, tungsten, vanadium, yttrium, zinc, zirconium, or a combination thereof.

14. The method according to claim 13, wherein the metal-film substrate comprises gold.

15. The method of claim 10, wherein the thiolated molecules comprise thiolated-nucleic acids, thiolated-peptides, thiolated-lipids, thiolated-sugars or any combination thereof.

16. The method of claim 11, wherein the maleimide-modified molecules comprise maleimide-modified oligonucleotides, maleimide-modified polymeric DNA, maleimide-modified peptides, maleimide-modified proteins, maleimide-modified lipids, and maleimide-modified sugars.

17. The method according to claim 12, wherein the PMPMS comprises a 10 mM solution of PMPMS in toluene.
